(19) United States
(12) Patent Application Publication
(10) Pub. No.: US 2025/0082752 A1
(43) Pub. Date: Mar. 13, 2025

(54) COMPOUNDS, TARGETS, AND METHODS FOR MODULATING LYTIC GRANULE CONVERGENCE IN CYTOTOXIC CELLS TO PROMOTE BYSTANDER KILLING IN CELLULAR THERAPIES

(71) Applicant: THE TRUSTEES OF COLUMBIA UNIVERSITY IN THE CITY OF NEW YORK, New York, NY (US)

(72) Inventors: Jordan Scott ORANGE, New York, NY (US); Luis Alberto PEDROZA, New York, NY (US); Michael LAM, New York, NY (US); Yu LI, New York, NY (US); Frédérique VAN DEN HAAK, New York, NY (US)

(21) Appl. No.: 18/893,711

(22) Filed: Sep. 23, 2024

Related U.S. Application Data
(63) Continuation-in-part of application No. PCT/US2023/016141, filed on Mar. 23, 2023.
(60) Provisional application No. 63/341,291, filed on May 12, 2022.
(60) Provisional application No. 63/322,938, filed on Mar. 23, 2022.

(51) Int. Cl. A61K 39/00; C12N 5/0783; C12N 9/22; C12N 15/11; C12N 15/90

(52) U.S. Cl. CPC A61K 40/30; A61K 40/15; A61K 40/31; C12N 5/0646; C12N 9/22; C12N 15/11; C12N 15/907; C12N 2310/20; C12N 2501/999

(57) ABSTRACT

Disrupting convergence of lytic granules produced by cytotoxic lymphocytes allows non-directional degranulation, which improves and broadens killing efficiency of the cytotoxic cells in pathogenic environments such as when used for cancer therapy. Accordingly, methods of inducing multidirectional degranulation by cytotoxic effector cells in a tumor microenvironment, methods of treating a tumor, and related therapeutic composition are described.

### CROSS-REFERENCE TO RELATED APPLICATION

This application claims priority to and is a continuation-in-part of International Application No. PCT/US2023/016141, filed on Mar. 23, 2023, which claims the benefit of U.S. Provisional Application No. 63/322,938 filed Mar. 23, 2022 and of U.S. Provisional Application No. 63/341,291 filed May 12, 2022, the contents of which are hereby incorporated by reference.

### GOVERNMENT SUPPORT

This invention was made with government support under grant AI067946 awarded by the National Institutes of Health (NIH). The government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Immune defense against intracellular pathogens and tumors by cytotoxic effector cells, which include NK cells and cytotoxic T lymphocytes (CTLs), is fundamental to human health. Cytotoxic cells express high levels of cytolytic effector molecules and use them to kill diseased cells on direct contacts. These effector molecules are safely processed and stored inside lysosome-related organelles, a type of vesicle known as lytic granules, which are specialized for secretion. Upon recognition of a target, a cytotoxic cell establishes an immunological synapse to initiate a series of tightly regulated downstream events. Eventually, lytic granules are mobilized towards presynaptic membrane, dock and fuse with the presynaptic membrane, and then extrude their cytotoxic contents into the synaptic cleft between the cytotoxic cell and target cell, which eventually leads to the destruction of the target cell.

During the mobilization of lytic granules, one remarkable phenomenon is their convergence to the microtubule-organizing center (MTOC). Driven by dynein motors, lytic granules move towards minus-end of microtubule and accumulate around MTOC. This is followed by polarization of the lytic granules along with the MTOC to the synapse formed with the target cell. Among cells that contain lysosome-related organelles, cytotoxic effector cells are the only known cells to converge their granules before secreting the granule contents onto target cells. This unique feature has been proven to have profound biological significance. When the convergence is chemically blocked, cytotoxic cells were found to degranulate multi-directionally and the bystander killing of neighboring innocent cells was significantly increased. Therefore, lytic granule convergence serves as a prerequisite step enabling cytotoxic cells to compress their cytolytic cargo and allows for focused release of effector molecules to directionally trigger target cell lysis.

When considering how a cytotoxic cell may interrogate a complex tissue mostly consisted of healthy cells to “seek and destroy” diseased cells, the convergence mechanism emerges as fundamental for preserving the healthy tissue microenvironment. In certain scenarios, however, a more diffused killing can be wanted and overall beneficial to therapeutic purposes. For instance, tumor-infiltrating lymphocytes are often inhibited by hostile microenvironment in solid tumors. Functional analyses have revealed a dampened activation and degranulation in cytotoxic subsets. By promoting multi-directional degranulation within a tumor environment, suppressed anti-tumor cytotoxicity can be unleashed by maximally taking advantage of each degranulation event and killing additional tumor cells through enhanced collateral damage.

### SUMMARY

Disclosed herein are methods of inducing multidirectional degranulation by cytotoxic effector cells (for example, NK cells or T cells) within a tumor microenvironment, wherein the tumor microenvironment comprises a target tumor cell and bystander cells. In some aspects, the tumor microenvironment is that of a solid tumor, such as osteosarcoma or lymphoma. The method comprises inhibiting the function of at least one vesicle modifying protein in the cytotoxic effector cells and providing the inhibited cytotoxic effector cells to the tumor microenvironment, whereby the bystander cells and the target tumor cell are killed. The at least one vesicle modifying protein is selected from the group consisting of: a trans-Golgi golgin, Brefeldin A-Inhibited Guanine Nucleotide-Exchange Protein 1 (BIG1), VASP, ARL1, Rab Geranyl-Geranyltransferase Subunit Alpha (RABGGTA), and a cytoskeleton-related protein. In some aspects, the trans-Golgi golgin is selected from the group consisting of: GCC1, GCC2, Golgin-97, and Golgin-245. In some aspects, the cytoskeleton-related protein is selected from the group consisting of: a septin (for example, Septin-7 (SEPT7) or Septin-2), HAP40, CCDC84, HKRP3, and beta actin (ACTB). In some implementations, the method further comprises administering to the cytotoxic effector cells a small molecule inhibitor prior to providing the inhibited cytotoxic effector cells to the tumor microenvironment. The small molecule inhibitor is selected from the group consisting of: 1-oleoyl lysophosphatidic acid (LPA), CFI-400945, Centrinone, Ciliobrevin-D, Dynapyrazole A, Compound 3016, Compound 5814, Compound 7779, Compound 3977, and a derivative thereof. In some aspects, the small molecule inhibitor is a dynein inhibitor, wherein the dynein inhibitor is selected from the group consisting of: Ciliobrevin-D, Dynapyrazole A, Compound 3016, and a derivative thereof.

In some embodiments, the function of at least one vesicle modifying protein is inhibited by knocking down expression of the at least one vesicle modifying protein in the cytotoxic effector cells, for example, with an RNA silencing technique. In some implementations, the expression of the at least one vesicle modifying protein is knocked down using an shRNA. Such shRNAs are commercially available, such as from Santa Cruz Biotechnology.

In other embodiments, the function of at least one vesicle modifying protein is inhibited by knocking in expression of the at least one vesicle modifying protein in the cytotoxic effector cells, for example by transfecting the cytotoxic effector cells with a plasmid comprising a gene encoding a mutated, preferably non-functional, vesicle modifying protein. In particular embodiments, for example, the function of GCC2 is inhibited by knocking in expression of the protein using a plasmid containing a single point mutation at residue 1608 (glutamic acid to glycine).

In still other embodiments, the function of at least one vesicle modifying protein is inhibited by knocking out expression of the at least one vesicle modifying protein in the cytotoxic effector cells, for example using a genome editing method. In some aspects, the genome editing method is a CRISPR-mediated genome editing method. In some aspects, the genome editing method involves a zinc finger nuclease (ZFN) or a transcription activator-like effector-based nuclease (TALEN). In some aspects, the genome editing method is a CRISPR-Transposon system for DNA modification as disclosed, e.g., in WO/2020/181264 and WO/2022/261122, which are incorporated herein in their entirety by reference thereto. In some aspects, the expression of the at least one vesicle modifying protein in the cytotoxic effector cells is knocked out using CRISPR-mediated genome editing of a trans-Golgi golgin, BIG1, Septin-7, HAP40, CCDC84, HKRP3, VASP, ARL1, RABGGTA, or ACTB. The guide sequence (used interchangeably herein as sgRNA or gRNA) in the CRISPR-mediated genome editing system targeting the genes encoding the aforementioned vesicle modifying proteins is commercially available, for example from Horizon, a PerkinElmer Company. The method of knocking out expression of the at least one vesicle modifying protein in the cytotoxic effector cells using the CRISPR-mediated genome editing method comprises providing a cytotoxic effector cell, and introducing into the cell (i) a nucleic acid that comprises a nucleotide sequence of a CRISPR-Cas system guide RNA (gRNA), which hybridizes to a portion of the nucleotide sequence that encodes the at least one vesicle modifying protein (e.g., GCC2, CCDC84, Septin-7, HKRP3, HAP40, VASP, ARL1, RABGGTA, or ACTB), and (ii) a Cas endonuclease (e.g., Cas9 or Cpf1). The nucleic acid that comprises a nucleotides sequence of a CRISPR-Cas system gRNA and a Cas nuclease may, for example, be encoded on the same nucleic acid or on different nucleic acids or introduced into the cell as a pre-formed ribonucleoprotein complex. In some embodiments, the portion at the nucleotide sequence to which the gRNA hybridizes consists of 18-22 nucleotides.

In yet other embodiments, the function of at least one vesicle modifying protein is inhibited by administering to the cytotoxic effector cells a small molecule inhibitor selected from the group consisting of: LPA, CFI-400945, Centrinone, Compound 5814, Compound 7779, Compound 3977, and a derivative thereof. Accordingly, in embodiments of the method of inducing multidirectional degranulation by cytotoxic effector cells (for example, NK cells) within a tumor microenvironment, the method comprises providing cytotoxic effector cells; administering to the cytotoxic effector cells a small molecule inhibitor selected from the group consisting of: LPA, CFI-400945, Centrinone, Compound 5814, Compound 7779, Compound 3977, and a derivative thereof to produce inhibited cytotoxic effector cells; and administering the inhibited cytotoxic effector cells to the tumor microenvironment whereby the bystander cells and the target tumor cell are killed.

Also disclosed herein are methods of treating a tumor in a subject. The method comprises inhibiting the function of at least one vesicle modifying protein in the cytotoxic effector cells, wherein the at least one vesicle modifying protein is selected from the group consisting of: a trans-Golgi golgin, BIG1, VASP, ARL1, RABGGTA, and a cytoskeleton-related protein. The inhibited cytotoxic effector cells are then administered to the subject. Examples of trans-Golgi golgins for inhibition include GCC1, GCC2, Golgin-97, and Golgin-245. Examples of cytoskeleton-related protein include a septin (for example, Septin-7), HAP40, CCDC84, HKRP3, and ACTB. In some implementations, the method further comprises administering to the cytotoxic effector cells a small molecule inhibitorprior to providing the inhibited cytotoxic effector cells to the tumor microenvironment. The small molecule inhibitor is selected from the group consisting of: LPA, CFI-400945, Centrinone, Ciliobrevin-D, Dynapyrazole A, Compound 3016, Compound 5814, Compound 7779, Compound 3977, and a derivative thereof. In some aspects, the small molecule inhibitor is a dynein inhibitor, wherein the dynein inhibitor is selected from the group consisting of: Ciliobrevin-D, Dynapyrazole A, and Compound 3016.

In some embodiments, the function of at least one vesicle modifying protein is inhibited by knocking down expression of the at least one vesicle modifying protein in the cytotoxic effector cells, for example, with shRNA. In other embodiments, the function of at least one vesicle modifying protein is inhibited by knocking out expression of the at least one vesicle modifying protein in the cytotoxic effector cells, for example using a CRISPR-mediated genome editing method. In some aspects, the expression of the at least one vesicle modifying protein in the cytotoxic effector cells is knocked out using CRISPR-mediated genome editing of a trans-Golgi golgin, BIG1, Septin-7, HAP40, CCDC84, HKRP3, RABGGTA, or ACTB. In still other embodiments, the function of at least one vesicle modifying protein is inhibited by administering to the cytotoxic effector cells a small molecule inhibitor selected from the group consisting of: LPA, CFI-400945, Centrinone, Compound 5814, Compound 7779, Compound 3977, and modifications thereof.

The method of knocking out expression of the at least one vesicle modifying protein in the cytotoxic effector cells using the CRISPR-mediated genome editing method comprises providing a cytotoxic effector cell, and introducing into the cell (i) a nucleic acid that comprises a nucleotide sequence of a CRISPR-Cas system gRNA, which hybridizes to a portion of the nucleotide sequence that encodes the at least one vesicle modifying protein (e.g., GCC2, CCDC84, Septin-7, HKRP3, HAP40, VASP, ARL1, RABGGTA, or ACTB), and (ii) a Cas endonuclease (e.g., Cas9 or Cpf1). The nucleic acid that comprises a nucleotides sequence of a CRISPR-Cas system gRNA and a Cas nuclease may, for example, be encoded on the same nucleic acid or on different nucleic acids, or introduced into the cell as a pre-formed ribonucleoprotein complex. In some embodiments, the portion at the nucleotide sequence to which the gRNA hybridizes consists of 18-22 nucleotides. In particular embodiments, the function of GCC2 is inhibited by knocking out expression of this gene using CRISPR-mediated genome editing methods.

Further disclosed herein are compositions comprising inhibited cytotoxic effector cells. The inhibited cytotoxic effector cells have reduced function of at least one vesicle modifying protein compared to uninhibited cytotoxic effector cells.

Accordingly, in some embodiments, the compositions comprises cytotoxic effector cells and a system for knocking down, knocking out, or knocking in expression of the at least one vesicle modifying protein in the cytotoxic effector cells. In some aspects, the composition comprises a CRISPR construct for knocking out expression of at least one vesicle modifying protein, for example, GCC1, GCC2, Golgin-97, Golgin-245, Septin-7, HAP40, CCDC84, HKRP3, VASP, ARL1, RABGGTA, or ACTB. Accordingly, the composition comprises gRNA that hybridizes to a portion of the nucleotide sequence that encodes the at least one vesicle modifying protein and a Cas endonuclease. In other aspects, the composition comprises an shRNA construct for knocking down expression of at least one vesicle modifying protein, for example, GCC1, GCC2, Golgin-97, Golgin-245, Septin-7, HAP40, CCDC84, HKRP3, VASP, ARL1, RABGGTA, or ACTB. In still other aspects, the composition comprises a plasmid comprising a mutated version of the gene encoding a vesicle modifying protein for knocking in expression of the vesicle modifying protein. The mutated version of the gene or gene product produces a non-functional vesicle modifying protein or a vesicle modifying protein with limited function, thus inhibiting the function of the vesicle modifying protein. For example, in a particular embodiment, the composition comprising a plasmid encoding GCC2 with a point mutation at residue 1608 and cytotoxic effector cells, wherein transfection of the cytotoxic effector cells with the plasmid produces cytotoxic effector cells with inhibited GCC2 function.

In other embodiments, the composition comprises inhibited cytotoxic effector cells that have at least one gene knocked out. In some aspects, the at least one gene is selected from the group consisting of: GCC1, GCC2, Golgin-97, Golgin-245, Septin-7, HAP40, CCDC84, HKRP3, VASP, ARL1, Rab Geranyl-Geranyltransferase Subunit Alpha (RABGGTA), and beta actin (ACTB). In such embodiments, the at least one gene is knocked out with a gRNA that hybridizes to the gene and a Cas endonuclease. In still other embodiments, the composition comprises inhibited cytotoxic effector cells that have at least one gene knocked down. In some aspects, the at least one gene selection from the group consisting of: GCC1, GCC2, Golgin-97, Golgin-245, Septin-7, HAP40, CCDC84, HKRP3, VASP, ARL1, Rab Geranyl-Geranyltransferase Subunit Alpha (RABGGTA), and beta actin (ACTB). In such embodiments, the at least one gene is knocked down with an shRNA construct for knocking down expression of the at least one gene. In yet another embodiment, the composition comprises inhibited cytotoxic effector cells that expresses a mutated vesicle modifying protein. In some aspects, the inhibited cytotoxic effector cells has been transfected with a plasmid encoding the mutated vesicle modifying protein.

In yet other embodiments, the composition comprises inhibited cytotoxic effector cells that have been treated by a small molecule inhibitor. In still other embodiments, the composition comprises cytotoxic effector cells and a small molecule. The small molecule inhibitor is selected from the group consisting of: LPA, CFI-400945, Centrinone, Ciliobrevin-D, Dynapyrazole A, Compound 3016, Compound 5814, Compound 7779, Compound 3977, and a derivative thereof.

### BRIEF DESCRIPTION OF FIGURES

The patent or application file contains at least one drawing executed in color. Copies of this patent or patent application publication with color drawing(s) will be provided by the Office upon request and payment of the necessary fee.

FIGS. 1A-1C depict, in accordance with certain embodiments, collapsing of cell strata triggered by phase transition of responsive hydrogel (PNIPAM).

FIGS. 2A-2C depict, in accordance with certain embodiments, collapsing of spheroid triggered by phase transition of responsive hydrogel (PNIPAM).

FIG. 3 depicts, in accordance with certain embodiments, three-view drawings of the lower mold.

FIG. 4 depicts, in accordance with certain embodiments, three-view drawings of the upper mold.

FIG. 5 depicts, in accordance with certain embodiments, three-view drawings of the demolding tool.

FIG. 6 depicts, in accordance with certain embodiments, assembly of the lower mold and the upper mold.

FIG. 7 depicts, in accordance with certain embodiments, a schematic of making agarose lower die using the lower mold and the upper mold.

FIG. 8 depicts, in accordance with certain embodiments, a schematic of removing gel assembly from the lower mold using the demolding tool.

FIG. 9 depicts photographs of the collapse of cell strata under roof temperature within 10 minutes in accordance with certain embodiments.

FIGS. 10A and 10B show that dynein inhibition leads to lytic granule dispersion. YTS cells were treated with DMSO or one of three different dynein inhibitors (Ciliobrevin-D, Dynapyrazole-A, or Compound 3016) and then incubated on an activating glass surface for 30 min. Cells were fixed, stained for a-tubulin, F-actin, perforin, and DNA (DAPI), and imaged via spinning disc confocal microscopy. Examples of individual cells (top) showing all stains and a broader field (bottom) at lower magnification showing only perforin to give an impression of the degree of granule convergence in a field of cells (FIG. 10A). The effect of dynapyrazole-A (blue line) was more durable in sustaining deconvergence after NK cell activation on an activating glass surface than Ciliobrevin-D (red line) at different times after being removed from media (FIG. 10B). Points show mean+SD of 3 independent experiments measuring >20 cells each. Neither inhibitor, however, promotes sustained deconvergence after the inhibitor has been removed from media.

FIGS. 11A-11H depict exemplary results related to mean granule distance (MGD) and automated estimated mean granule distance (eMGD). FIG. 11A depicts an exemplary high-resolution confocal imaging of lytic granules (labeled by perforin) and tubulin, which allows for automated intensity threshold-based identification of granules as shown in FIG. 11B (outlines indicate for granules and the microtubule-organizing center (MTOC)). FIG. 11C depicts the equation for calculating MGF using x and y coordinates of each granule relative to the MTOC. FIG. 11D depicts the 384-well glass bottom plate and InCell Analyzer (20× objective, 0.45NA) used in a platform for an automated, high-throughput method for estimating MGD. FIG. 11E is an exemplary image of a fixed NK cell from the screen (labeled with perforin and tubulin) to which the Euclidean method for assessing granule localization relative to a total cell, tubulin-defined footprint of FIG. 11F can be applied. eMGD is then calculated using a Matlab script and the formula shown in FIG. 11G. The script pixelates the image, retrieves the MTOC position from the a-tubulin channel (xc, yc), and then traverses all pixels in the perforin channel, first calculating the intensity of each pixel f(i, j) and then defining its distance from the MTOC. Pixels are then summed to generate eMGD. FIG. 11H shows an exemplary comparison of MGD and eMGD using YTS cells activated on glass via CD18/CD28 for 30 minutes using images obtained from a confocal microscope (MGD) and from the GE InCell Analyzer (eMGD). Increase between DMSO and Dynapyrazole-A (DPZ-A) treatment for significant for both MGD and eMGD (*p<0.01).

FIG. 12 show that, in an activating surface with CD28/CD18, YTS cells treated with Dynapyrazole-A shows a high lytic granule dispersion by increase the granule distance to MTOC (μm).

FIG. 13 shows that Dynapyrazole-A-pretreated YTS cells kill bystander K652 cells. The graph is representative of three independently performed biological repeats.

FIGS. 14A and 14B show that YTS cells stimulated with LPA results in Golgi fragmentation (FIG. 14A) and granule dispersion (FIG. 14B). The results of a representative Cr15 killing assay on YTS cell treated with LPA are shown in FIG. 14C.

FIG. 15 shows the changes in the number of MTOC caused by treating YTS cells with Centrinone (5 nM) or CFI400495 (50 nM), as measured by the number of α-tubulin hyper densities.

FIGS. 16A-16D demonstrates that Centrinone and CFI400495 induce degranulation in treated cytotoxic cells. FIG. 16A show that treatment of Centrinone or CFI400495 increases degranulation as represented by CD107a expression after conjugation with 721.221 target cells. FIGS. 16B-16D are exemplary confocal microscopy images that show more disperse granules in YTS cells after treatment with Centrinone or CFI400495.

FIGS. 17A-17D demonstrates that HkRP3 is required for lytic granule convergence. HkRP3 was deleted from YTS cells via CRISPR/Cas9 gene editing. Western blot analysis confirmed the absence of HkRP3 protein in edited cells relative to a tubulin loading control (FIG. 17A). HKRP3-deleted YTS cells lysed ⁵¹Cr-labeled 721.221 target less efficiently in a 4-hour assay (FIG. 17B). Exemplary confocal images of control and HkRP3-deleted YTS cells (top cells) conjugated with 721.221 target cells (bottom cells) fixed and stained for perforin, «-tubulin, and DNA are shown in FIG. 17C. Lytic granules are converged in control cells and dispersed in the HkRP3-deleted YTS cells. The MTOC is marked by a white star. FIG. 17D shows quantification of lytic granule convergence. MGD was measured for 721.211-conjugated control and HkRP3-deleted YTS cells. HkRP3-deletion prevents activation-induced convergence (*=p<0.01).

FIG. 18 confirms trans-golgi golgins were knocked down or knocked out in YTS lines.

FIG. 19 shows the results of Cr51 killing assay on YTS cells knocked down for golgins.

FIGS. 20A and 20B show inhibiting golgins cause deconvergence of granules as represented by increase granule distance to MTOC in YTS cells.

FIGS. 21A-21E depicts exemplary fluorescent microscopy images providing visual depiction of granule dispersion.

FIG. 22 shows the results of the CD107a degranulation test for golgins-deficient YTS cells.

FIGS. 23A-23C shows the killing efficiency of bystander cells by GCC2 KO and Golgin 97 KD YTS cells. The bystander cells tested are K562 cells (FIG. 23A, n=5), LM7 cells (FIG. 23B, n=3), and 143B cells (FIG. 23C, n=3).

FIG. 24 confirms trans-golgi golgins were knocked down or knocked out in NK92 cells.

FIG. 25 show inhibiting golgins cause deconvergence of granules as represented by increase granule distance to MTOC in NK92 cells.

FIGS. 26A-26C depicts exemplary fluorescent microscopy images providing visual depiction of granule dispersion.

FIG. 27 shows the results of the CD107a degranulation test for golgins-deficient NK92 cells.

FIG. 28 shows the killing efficiency of bystander Raji cells by GCC2 KO and Golgin 97 KD NK92 cells. n=3

FIG. 29 confirms GCC2 was deleted in ex vivo expanded human NK cells using CRISPR/Cas9.

FIG. 30 shows editing ex vivo NK cells (eNK) to be deficient in golgins cause deconvergence of granules as represented by increase granule distance to MTOC.

FIGS. 31A-31C depicts exemplary fluorescent microscopy images providing visual depiction of granule dispersion.

FIG. 32 shows the results of the CD107a degranulation test for golgins-deficient eNK cells.

FIGS. 33A-33C show inhibiting BIG1 cause deconvergence of granules as represented by increased granule distance to MTOC in YTS cells.

FIGS. 34A and 34B depicts exemplary fluorescent microscopy images providing visual depiction of granule dispersion.

FIG. 35 depicts the results of a representative Cr15 killing assay on YTS cell with BIG1 expression knocked down.

FIG. 36 confirms Septin-7 was deleted in NK92 cells using CRISPR/Cas9.

FIGS. 37A and 37B show the degranulation of SEPT7 KO NK92 cells. Tested with 721.211 cells and K562 cells at 30 mins (FIG. 37A) and at 60 mins (FIG. 37B).

FIG. 38 depicts the results of a representative Cr15 killing assay on NK92 cell with SEPT7 expression knocked out.

FIG. 39 show inhibiting SEPT7 cause deconvergence of granules as represented by increased granule distance to MTOC in NK92 cells.

FIGS. 40A and 40B depicts exemplary fluorescent microscopy images providing visual depiction of granule dispersion.

FIGS. 41A-41C show that knocking down expression of HAP40 in YTS cells resulted in deconvergence as shown by increased granule distance to MTOC (FIG. 41A) and increased bystander killing (FIG. 41C). FIG. 41B depicts representative results of the CD107a degranulation test on YTS cell with HAP40 expression knocked down.

FIGS. 42A-42C show that knocking out expression of CCDC84 in YTS cells reduced convergence of granules without negatively affecting killing efficiency of the edited YTS cells. FIGS. 42A and 42B are respectively fluorescent microscopy images normal and edited YTS cells, with the granules shown in red and tubulin shown in green. Each hyper density is counted as 1 MTOC, arrows showed cells with more than one mTOC. FIG. 42C is a graph comparing the number of MTOCs.

FIGS. 43A and 43B respectively show the results of a representative Cr15 killing assay on NK92 cell with CCDC84 expression knocked out and a graph comparing the distance of the granules to the MTOC during the conjugation with target cell.

FIGS. 44A-44D depict the schematics illustrating the effect of changes in golgins expression and Golgi structure and MTOC structure on convergence of lytic granules.

FIG. 45 depicts the schematics illustrating the effect of changes in septin expression on convergence of lytic granules.

FIG. 46A confirms knock down of VASP expression in YTS cells by siRNA, and FIG. 46B demonstrates that such cells have increased distance between the lytic granules and MTOC, which suggests an increase on granule dispersion.

FIG. 47A shows that knocking out expression of ARL1 in NK92 cells increased granule dispersion. However, ARL1-deficient NK cells did not show an increased degranulation (as represented by a CD107a expression) after conjugation with 721.221 target cells or K592 target cells (FIG. 47B).

FIG. 48 shows that chimeric antigen receptor T cells and chimeric antigen receptor NK cells exhibit convergence. PSCA: Prostate Stem Cell Antigen; HER-2: Human Epidermal Growth Factor 2; GD2: GD2 is a disialoganglioside expressed on tumors of neuroectodermal origin, including human neuroblastoma and melanoma, with highly restricted expression on normal tissues, principally to the cerebellum and peripheral nerves in humans.

FIG. 49 further confirms chimeric antigen receptor cytotoxic cells have converged lytic granules, as shown by the mean granule distance from MTOC.

FIGS. 50A and 50B demonstrate that the lytic granules in cell culture bearing chimeric antigen receptor are converged. FIG. 50A depicts a graph of the mean granule distance from MTOC of two types of chimeric antigen receptor cells without cytokine exposure or with exposure to IL17 and IL15. FIG. 50B is an exemplary fluorescent microscopy image of a cell in a chimeric antigen receptor cell culture with F-action, granzyme, and MTOC fluorescently labeled.

FIGS. 51A-51E show the effect of knocking in GCC2 (using GCC2 E1608G) in YTS cells and NK92 cells on degranulation and convergence of lytic granules. The CD107a degranulation test in YTS cells (FIG. 51A) and NK92 cells (FIG. 51B) show comparable level of degranulation after stimulation by 721.221 cells or 562 cells. The granule distance to MTOC in YTS cells is increased when GCC2 expression is knocked in after conjugation with a target cell, which suggests an increase granule dispersion (FIG. 51C). FIGS. 51D and 51E depict exemplary fluorescent microscopy images providing visual depiction of granule dispersion in parental YTS cells and knocked-in cells.

FIGS. 52A-52D show that knocking out expression of GCC2 in eNK cells reduced convergence of granules. FIG. 52A show diminished expression of GCC2 protein in eNK cells with expression of GCC2 knocked out. FIG. 52B depicts the results of a representative Cr15 killing assay 721.221 target cells compared to unedited eNK cells. FIG. 52C depicts a graph of the mean granule distance from MTOC of eNK GCC2 knockout cells. FIG. 52D shows the CD107a degranulation test after stimulation by 721.221 cells.

FIGS. 53A-53E show that knocking out expression of RABGGTA in NK cells increased killing efficiency and reduced convergence of granules. FIG. 53A show decreased expression of RABGGTA protein in knocked out NK cells (using CRISPR/Cas9) compared to parental cells. FIG. 53B depicts the results of a representative Cr15 killing assay 721.221 target cells compared to YTS parental NK cells showing efficient killing of target cells. FIG. 53C depicts a graph of the mean granule distance from MTOC of RABGGTA knockout cells suggesting increased granule dispersion. FIG. 53D shows the CD107a degranulation test after stimulation by 721.221 cells. FIG. 53E shows the killing efficiency of bystander cells by RABGGTA KD and YTS parental cells tested with bystander K562 cells.

FIGS. 54A-54E show that killing capacity in a single NK cell clone with expression of RABGGTA knocked out is reduced though granule dispersion is increased. FIG. 54A show no expression of RABGGTA protein is detected the single clone with RABGGTA knocked out using CRISPR/Cas9 compared to parental cells. FIG. 54B depicts the results of a representative Cr15 killing assay 721.221 target cells compared to YTS parental NK cells showing efficient killing of target cells. FIG. 54C depicts a graph of the mean granule distance from MTOC of RABGGTA knockout cells suggesting increased granule dispersion. FIG. 54D shows the CD107a degranulation test after stimulation by 721.221 cells. FIG. 54E shows the killing efficiency of bystander cells by RABGGTA KD and YTS parental cells tested with bystander K562 cells.

FIGS. 55A-55E show that knocking out expression of Beta Actin in NK cells increased killing efficiency and reduced convergence of granules. FIG. 55A demonstrate the effect of knocking out expression of Beta Actin in NK cell line with CRISPR/Cas9 resulting in loss of expression of Beta Actin protein compared to YTS parental cells. FIG. 55B depicts the results of a representative Cr15 killing assay 721.221 target cells compared to YTS parental NK cells indicating efficient killing of target cells by Beta Actin KO cells. FIG. 55C represents a graph of the mean granule distance from MTOC of Beta Actin knockout cells suggesting an increased granule dispersion. FIG. 55D shows the CD107a degranulation test after stimulation by 721.221 cells demonstrating increased degranulation in Beta Actin KO cells compared to YTS parental cells. FIG. 55E shows the killing efficiency of bystander cells by Beta Actin KO and YTS parental cells tested with bystander K562 cells.

FIG. 56 depicts the chemical structure of N-{1-[(4-chlorophenyl)methyl]piperidin-4-yl}prop-2-enamide (Compound 5814).

FIG. 57 depicts the chemical structure for the small molecule compound 1-(prop-2-enamido)-N-[(thiophen-2-yl)methyl]cyclohexane-1-carboxamide (Compound 7779),

FIG. 58 depicts the chemical structure of 1-[3-(4-fluorophenyl) prop-2-enoyl]-4-hydroxypyrrolidine-2-carboxylic acid (Compound 3977).

FIGS. 59A-59E are representative fluorescent microscopy images depicting lytic granule dispersion induced by treating NK cells with N-{1-[(4-chlorophenyl)methyl]piperidin-4-yl}prop-2-enamide (Compound 5814) at 5 μM for 30 minutes. FIGS. 59B-59E are magnified image of marked areas in FIG. 59A focusing on cells with dispersed granule. Perforin staining is shown in green, and alpha-tubulin is staining is shown in red.

FIGS. 60A-60E are representative fluorescent microscopy images depicting lytic granule dispersion induced by treating NK cells with 1-(prop-2-enamido)-N-[(thiophen-2-yl)methyl]cyclohexane-1-carboxamide (Compound 7779) at 5 μM for 30 minutes. FIGS. 60B-60E are magnified image of marked areas in FIG. 60A focusing on cells with dispersed granule. The position of the granules is indicated by perforin staining.

FIGS. 61A-61E are representative fluorescent microscopy images depicting lytic granule dispersion induced by treating NK cells with 1-[3-(4-fluorophenyl) prop-2-enoyl]-4-hydroxypyrrolidine-2-carboxylic acid (Compound 3977) at 5 μM for 30 minutes. FIGS. 61B-61E are magnified image of marked areas in FIG. 61A focusing on cells with dispersed granule. The position of the granules is indicated by perforin staining.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the term “tumor microenvironment” refers to the environment around a tumor, including the edge of the tumor, the surrounding blood vessels, immune cells, fibroblasts, signaling molecules, and the extracellular matrix.

As used herein, the term “vesicle modifying protein” refers to proteins with a role in transporting vesicles and to proteins that have a role in influencing vesicles and vesicle transport, such as cytoskeleton-related proteins. Exemplary vesicle modifying proteins include trans-Golgi golgins (for example, Golgin-97 (GOLGA1), Golgin-247 (GOLGA4 or G-247), GCC2, and GCC1), septins (for example, Septin-7 (SEPT7) and Septin-2 (SEPT2)), HAP40 (F8A1), Brefeldin A-Inhibited Guanine Nucleotide-Exchange Protein 1 (BIG1 or ARFGEP1), CENATAC (CCDC84), HKRP3, CLIP170, VASP, PYK, ARL1, Rab Geranyl-Geranyltransferase Subunit Alpha (RABGGTA), and beta actin (ACTB).

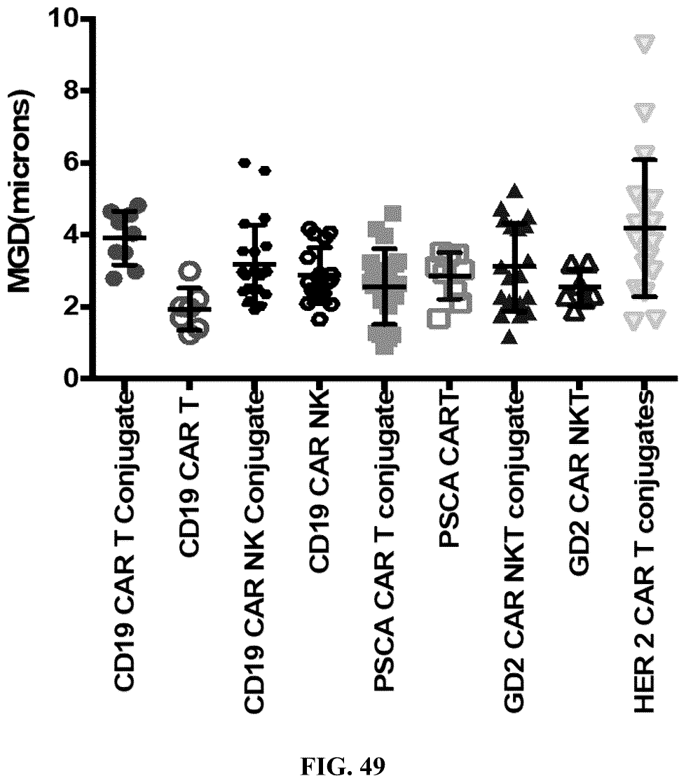

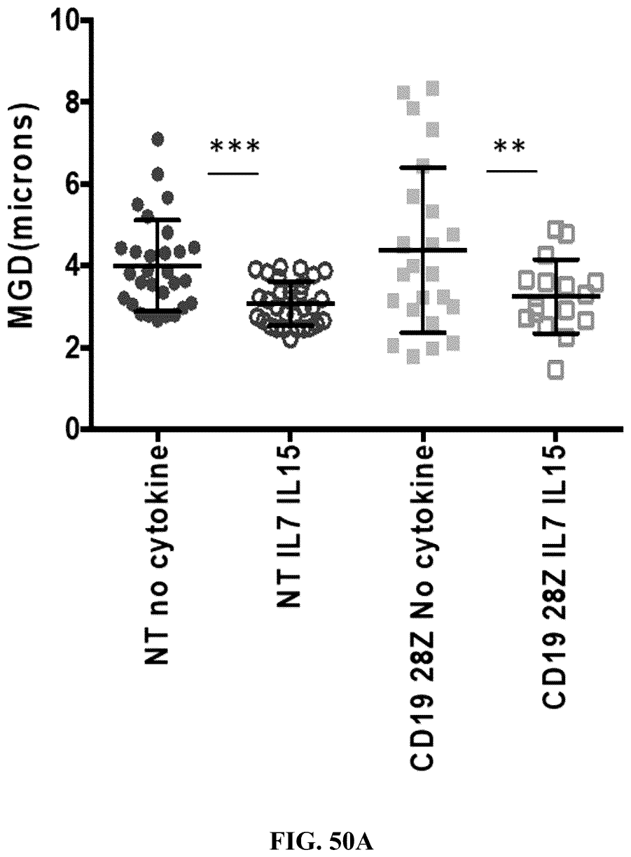

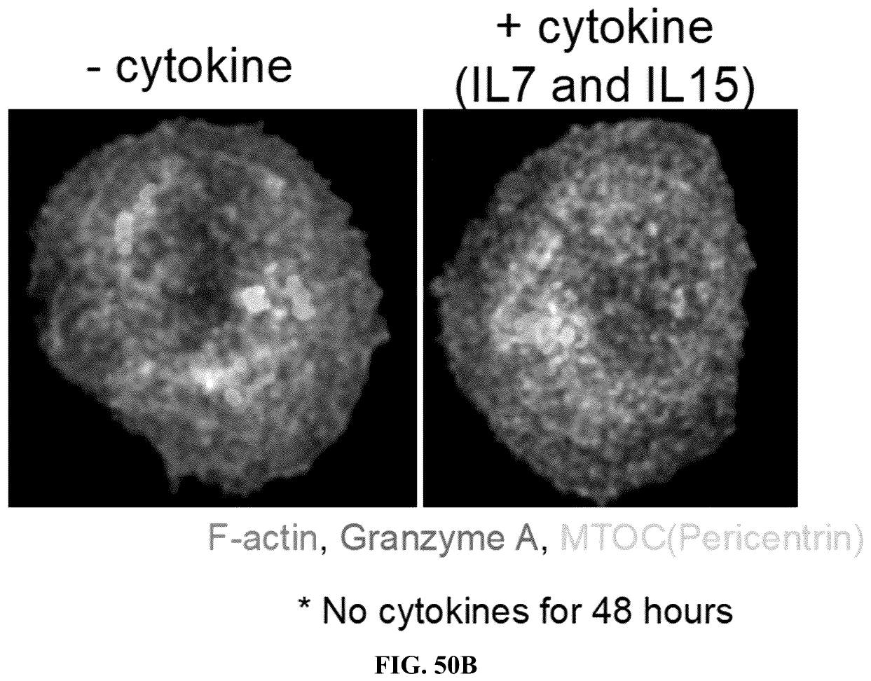

Disclosed herein are the use of known compounds for inducing multi-directional degranulation and bystander killing, for example, by interfering with convergence of lytic granules of cytotoxic effector cells. The cytotoxic effector cells may be natural killer (NK) cells, T cells, or natural killer T (NKT) cells. In some implementations, the cytotoxic effector cells comprise chimeric antigen receptors (CARs). As shown in FIGS. 49, 50A, and 50B, lytic granules in a variety of CAR T cells and CAR NK cells exhibit convergence. Thus, their therapeutic properties, such as in killing tumor cells as a cancer therapeutic, would benefit from inhibiting or reducing convergence of lytic granules.

Some of these compounds, such as dynapyrazole-A and Compound 3016, inhibit dynein motors that transport the lytic granules to the MTOC in cytotoxic effector cells. Other compounds, such as 1-oleoyl lysophosphatidic acid (LPA), induce Golgi fragmentation. Still other compounds, such as Centrinone and CFI400495, are a high affinity and selective PLK4 inhibitors that induce depletion of the centrosome and cell cycle arrest. Though Compound 5814, Compound 7779, and Compound 3977 were found to induce lytic granule dispersion, the exact mechanism of action is unknown. As shown in the Examples, the optimal working conditions for using these compounds to inhibit lytic granule dispersion have been identified and their effectiveness in inducing bystander killing of surrounding innocent cells validated the described tissue-mimicking model TheCOS. Thus, a method of inducing multidirectional degranulation within a tumor microenvironment comprising providing inhibited cytotoxic effector cells treated to tumor microenvironment is disclosed. The method comprises providing cytotoxic effector cells; administering to the cytotoxic effector cells a small molecule inhibitor selected from the group consisting of: LPA, CFI400945, Centrinone, Compound 5814, Compound 7779, Compound 3977, and a derivative thereof to produce inhibited cytotoxic effector cells; and administering the inhibited cytotoxic effector cells to the tumor microenvironment whereby bystander cells and target tumor cell in the tumor microenvironment are killed. Accordingly, therapeutic compositions comprising cytotoxic effector cells and the small molecule inhibitor is disclosed. In another embodiment, therapeutic composition comprising the inhibited cytotoxic effector cells that have been treated (as this inhibited) by the small molecule inhibitor is disclosed.

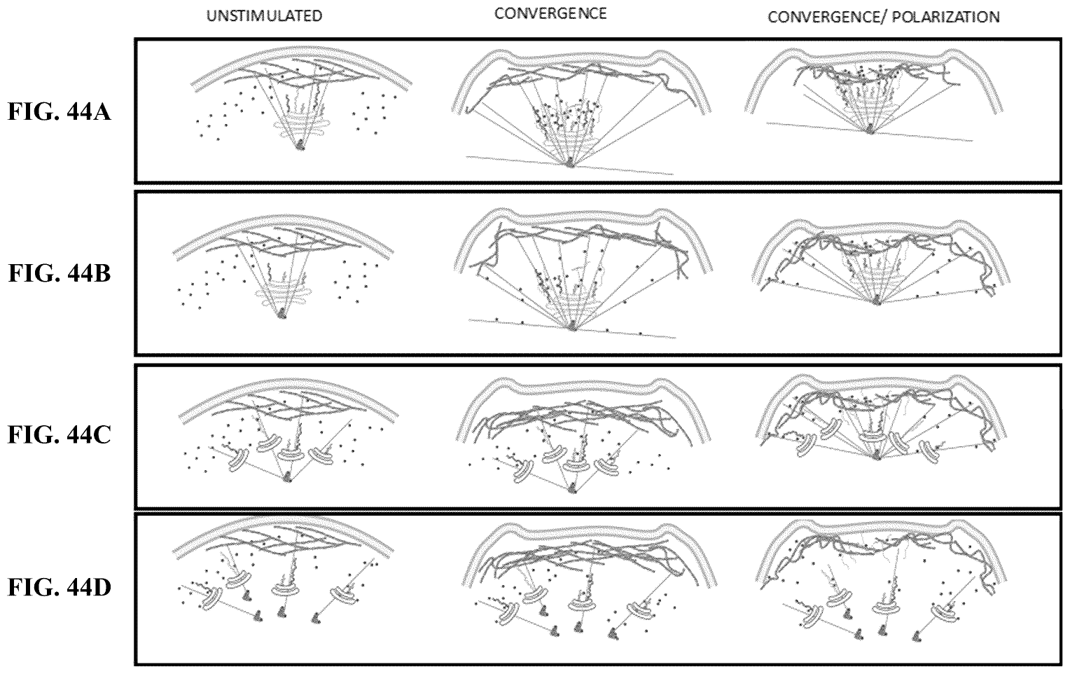

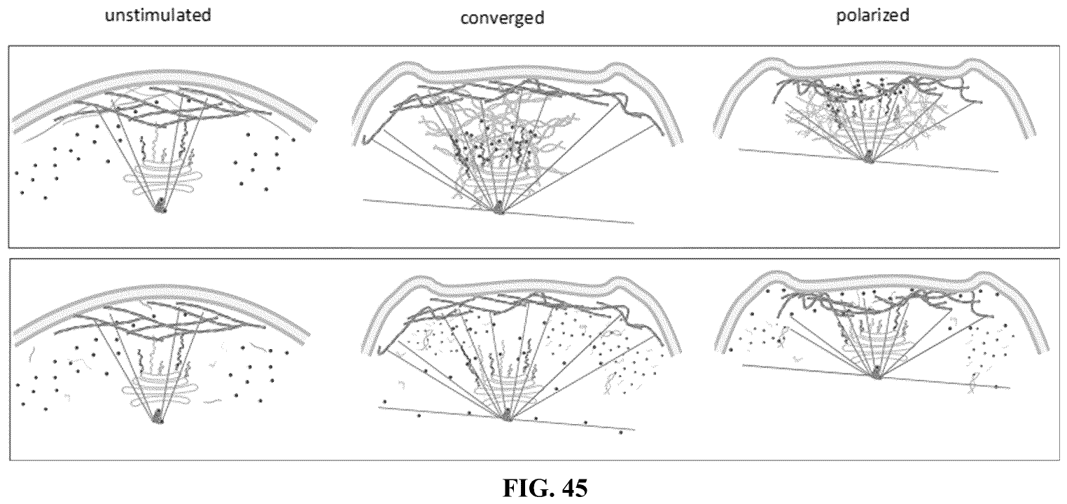

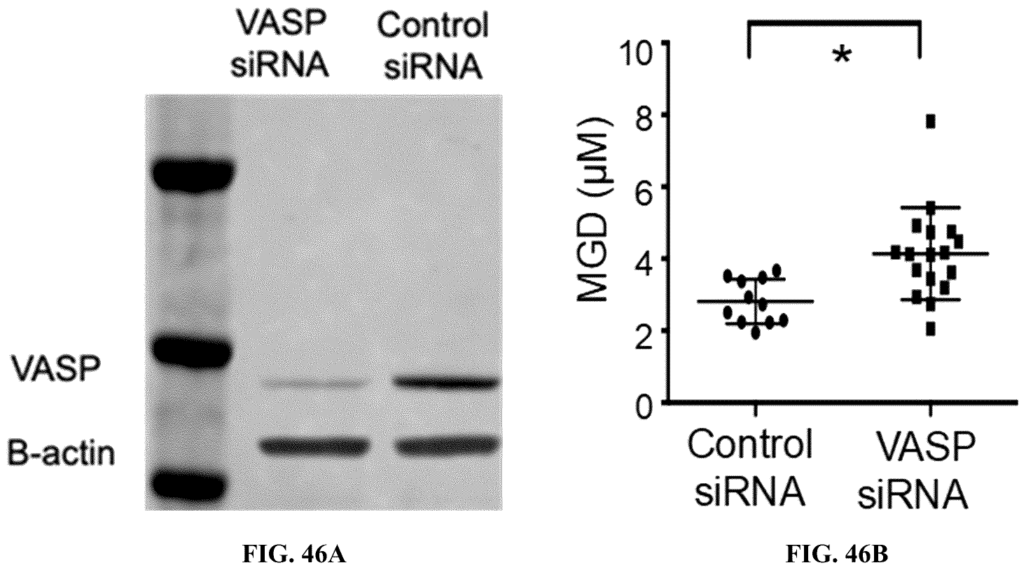

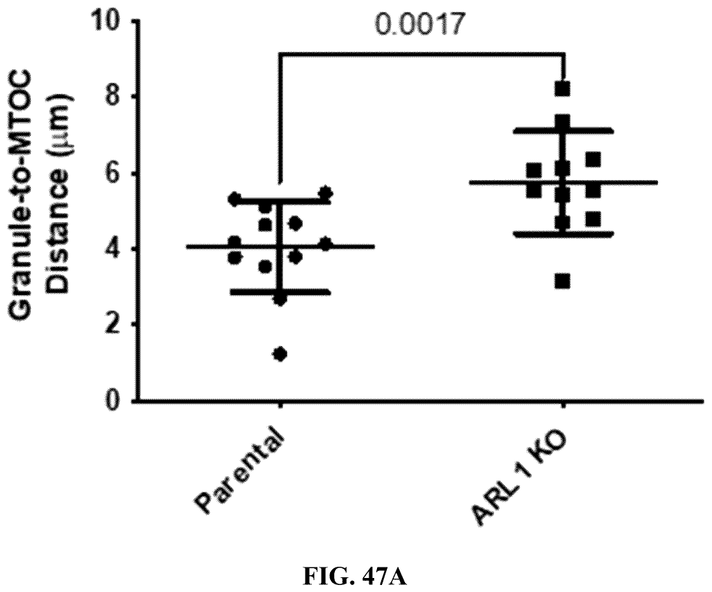

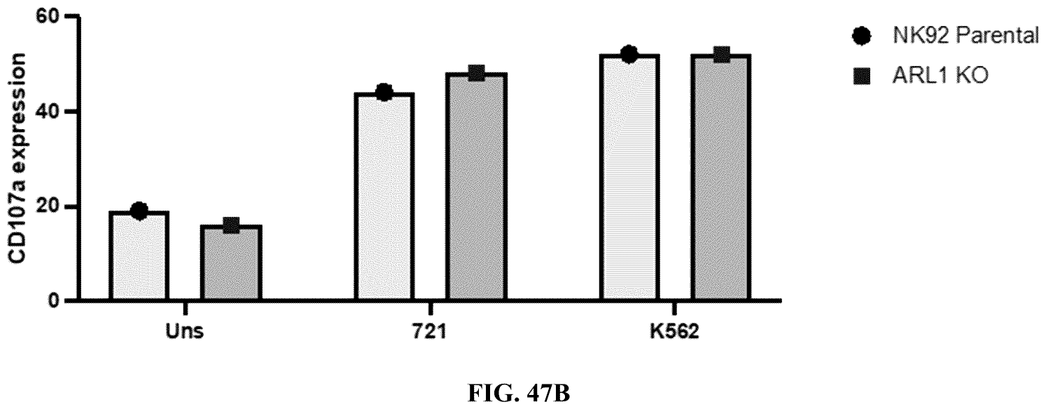

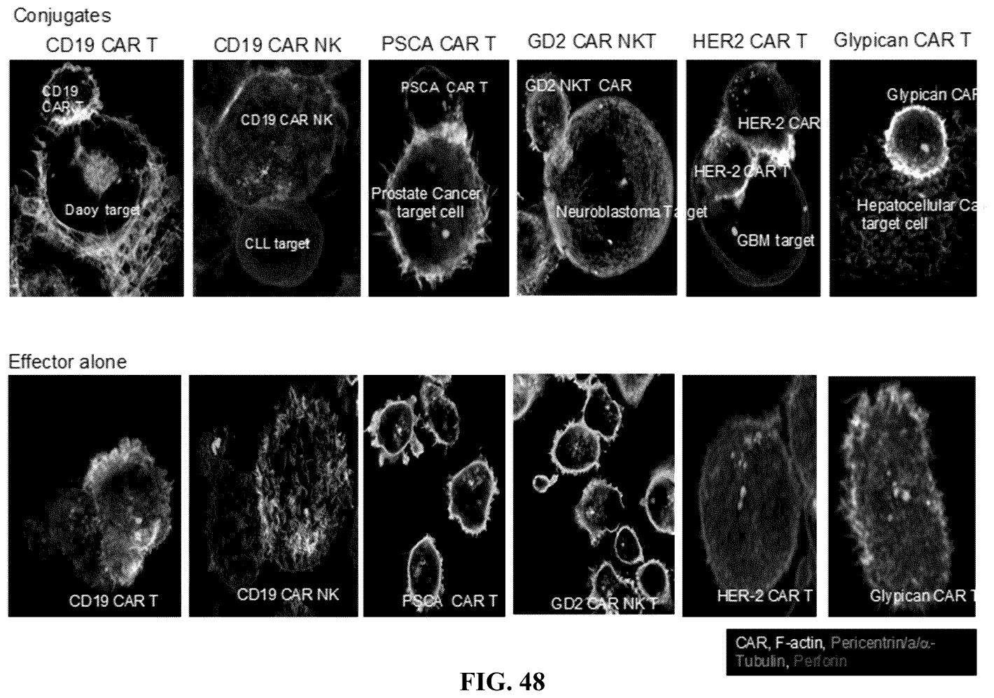

Also disclosed are cellular targets that promote lytic granule dispersion that serve as excellent targets for functionally dispersing lytic granules in NK cells and presumably in cytotoxic therapy cells. In some implementations, the cellular targets are vesicle modifying proteins. Thus, in some aspects, the cellular targets are selected from the group consisting of: a trans-Golgi golgin, a septin, BIG1, HAP40, CCDC84, HKRP3, VASP, ARL1, RABGGTA, and ACTB. In the absence of trans-Golgi golgins, convergence is partial (FIG. 44A). Few converged granules are polarized while the non-converged are randomly redirected through the tubulin network (FIG. 44B). When the Golgi Apparatus is fragmented, the convergence is deficient (no homogeneous Golgi around the mTOC), and granules are more randomly redirected through the tubulin network (FIG. 44D). When multiple MTOC are induced, the Golgi Apparatus is fragmented (secondary to pulled forces from the cell surface) and granules are more randomly redirected through the tubulin network (FIG. 44D). During convergence in healthy cells, septin interacts with other septins and septins interacts with lytic granules. Septin can from complexes such as filaments and acts as an inhibitory scaffolding cage interacting with lytic granules and other cytoskeletal components to hold lytic granules in place. During polarization, septin cytoskeleton is degraded only at the immune synapse releasing lytic granules for degranulation. However, when septin is knocked out and unable to form heterooligomeric complexes (such as filaments) and cannot form a scaffolding cage, dispersed granules result and are free to degranulate following activation (FIG. 45).

In certain implementations, the cellular targets are selected from the group consisting of: Golgin-97 (GOLGA1), Golgin-247 (GOLGA4 or G-247), GCC2, GCC1, Septin-7 (SEPT7), Septin-2 (SEPT2), HAP40 (F8A1), Brefeldin A-Inhibited Guanine Nucleotide-Exchange Protein 1 (BIG1 or ARFGEP1), CENATAC (CCDC84), HKRP3, CLIP170, VASP, PYK, ARL1, RABGGTA, and ACTB. GCC2 interacting proteins such as Rab9 and Rab 29, 44, 45 and 46 as well as CLASP1 are also cellular targets that promote lytic granule dispersion.

In view of these cellular targets that promote lytic granule dispersion, a method of inducing multidirectional degranulation by cytotoxic effector cells within a tumor microenvironment are disclosed. The method comprises inhibiting the function of at least one vesicle modifying protein in the cytotoxic effector cells. In some embodiments, the at least one vesicle modifying protein is selected from the group consisting of: a trans-Golgi golgin, BIG1, HKRP3, VASP, ARL1, RABGGTA, and a cytoskeleton-related protein. In some aspects, the trans-Golgi golgin is selected from the group consisting of: GCC1, GCC2, Golgin-97, and Golgin-245. In some aspects, the cytoskeleton-related protein is selected from the group consisting of a septin (for example Septin-7), HAP40, CCDC84, and ACTB. In some aspects, the function of at least one vesicle modifying protein is inhibited by knocking down, knocking out, or knocking in expression of the at least one vesicle modifying protein in the cytotoxic effector cells. For example, a trans-Golgi golgin, BIG1, Septin-7, HAP40, CCDC84, HKRP3, VASP, ARL1, RABGGTA, and/or ACTB is knocked out using CRISPR-mediated genome editing or knocked down using an RNA silencing method (for example, shRNA or siRNA). In other aspects, the function of at least one vesicle modifying protein is inhibited by administering an inhibitor selected from the group consisting of: LPA, CFI400945, Centrinone, Compound 5814, Compound 7779, and Compound 3977.

Accordingly, therapeutic compositions comprising construct for genetically altering the expression of vesicle modifying protein to inhibit its function in cytotoxic effector cells are disclosed and therapeutic cytotoxic effector cells, such as chimeric antigen receptor. In some embodiments, the therapeutic composition comprises a CRISPR construct for knocking out GCC1, GCC2, Golgin-97, Golgin-245, Septin-7, HAP40, CCDC84, HKRP3, VASP, ARL1, RABGGTA, or ACTB. In other embodiments, the therapeutic composition comprises RNA interference construct for knocking down GCC1, GCC2, Golgin-97, Golgin-245, Septin-7, HAP40, CCDC84, HKRP3, VASP, ARL1, RABGGTA, or ACTB are disclosed. Also disclosed herein are therapeutic compositions comprising inhibited cytotoxic effector cells such that these cells have reduced function of at least one vesicle modifying protein compared to uninhibited cytotoxic effector cells. In some aspects, the at least one vesicle modifying protein is selected from the group consisting of: a trans-Golgi golgin, BIG1, VASP, ARL1, Rab RABGGTA, and a cytoskeleton-related protein. In some implementations, the inhibited cytotoxic effectors are further inhibited by treatment with a small molecule inhibitor. Thus, in some embodiments, the therapeutic composition comprises the inhibited cytotoxic effector cells and a small molecule inhibitor selected from the group consisting of: LPA, CFI-400945, Centrinone, Compound 5814, Compound 7779, Compound 3977, Compound 5814, Compound 7779, Compound 3977, and a derivative thereof.

In certain implementations, the method of inducing multidirectional degranulation by cytotoxic effector cells within a tumor microenvironment comprises inhibiting the function of at least one vesicle modifying protein in the cytotoxic effector cells; and providing the inhibited cytotoxic effector cells to the tumor microenvironment, whereby bystander cells and target tumor cell in the tumor microenvironment are killed. In some aspects, the method further comprises administering to the cytotoxic effector cells a small molecule inhibitor prior to providing the inhibited cytotoxic effector cells to the tumor microenvironment. The small molecule inhibitor is selected from the group consisting of: LPA, CFI-400945, Centrinone, Ciliobrevin-D, Dynapyrazole A, Compound 3016, Compound 5814, Compound 7779, Compound 3977, and a derivative thereof. In some aspects, the small molecule inhibitor is a dynein inhibitor selected from the group consisting of: Ciliobrevin-D, Dynapyrazole A, Compound 3016, and a derivative thereof.

In some embodiments of the above-described methods induces multidirectional degranulation by cytotoxic effector cells within a solid tumor microenvironment, for example, that of osteosarcoma. In other aspects, the described methods induces multidirectional degranulation by cytotoxic effector cells within a lymphoblastic tumor microenvironment, such as that of a lymphoma. In particular implementations, the cytotoxic effector cells are NK cells.

Also described are a method of treating a tumor in a subject comprising: providing cytotoxic effect cells comprising chimeric antigen receptors specific to the tumor; inhibiting the function of at least one vesicle modifying protein in the cytotoxic effector cells; and administering the inhibited cytotoxic effector cells to the subject. In some embodiments, the at least one vesicle modifying protein is selected from the group consisting of: a trans-Golgi golgin, BIG1, HKRP3, VASP, ARL1, RABGGTA, and a cytoskeleton-related protein. In some aspects, the trans-Golgi golgin is selected from the group consisting of: GCC1, GCC2, Golgin-97, and Golgin-245. In some aspects, the cytoskeleton-related protein is selected from the group consisting of: a septin (for example Septin-7), HAP40, CCDC84, and ACTB. In some aspects, the function of at least one vesicle modifying protein is inhibited by knocking down or knocking out expression of the at least one vesicle modifying protein in the cytotoxic effector cells. For example, trans-Golgi golgin, BIG1, Septin-7, HAP40, CCDC84, and/or HkRP3 is knocked out using CRISPR-mediated genome editing or knocked down using RNA silencing techniques. In other aspects, the function of at least one vesicle modifying protein is inhibited by administering a small molecule inhibitor selected from the group consisting of: LPA, CFI400945, Centrinone, Compound 5814, Compound 7779, Compound 3977, and a derivative thereof. In some implementations, the method further comprises administering to the cytotoxic effector cells a dynein inhibitor prior to providing the inhibited cytotoxic effector cells to the tumor microenvironment, wherein the dynein inhibitor is selected from the group consisting of: Ciliobrevin-D, Dynapyrazole A, Compound 3016, and a derivative thereof.

In some embodiments of the above-described methods, the cytotoxic cells are treated with the inhibitor prior to delivery of the cells to the tumor microenvironment or the subject ex vivo or in vitro. In some implementations, the cytotoxic cells are exposed to the inhibitors within minutes, hours, or days of delivering the cells to the individual. However, in certain cases the cells are treated and then cryopreserved and stored for week, months, or years in that state prior to use. In some cases, the exposure of the cytotoxic cells to the inhibitor is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes or 1, 2, 3, 4, 5, 6, 7, 8, 9, 10, 11, 12, 13, 14, 15, 16, 17, 18, 19, 20, 21, 22, 23, or 24 hours. In some aspects, the exposure of the cytotoxic cells to the inhibitor is at least 1, 2, 3, 4, 5, 6, or 7 days. The maximum time that the cells are exposed to the agent is the entire culture expansion of the cells, although in specific cases to avoid difficulties in their division and further proliferation (for example, in the case of a small molecule inhibitor like a dynein inhibitor), the maximum time may be about 30 minutes.

In specific cases, the concentration of the inhibitor is 5 μM, 10 μM, 15 μM, 25 μM, 30 μM, 35 μM, 40 μM, 45 μM, 50 μM, 55 μM, 60 μM, 65 μM, 70 M, 75 μM, 80 μM, 85 μM, 90 μM, 95 μM, or 100 μM. A range of concentration may be 5 μM-100 UM, 5 μM-50 μM, 5 μM-25 μM, 10 μM-100 μM, 1 μM-75 μM, 10 μM-50 μM, 5 μM-25 μM, 25 μM-75 μM; 25 μM-50 μM; 50 μM-100 μM; 50 μM-75 μM; or 75 μM-100 μM. The inhibitor may be prepared as a stock solution in DMSO.

A cell model comprising responsive hydrogels with the ability to establish close intercellular contacts between designated cell groups in various three-dimensional geometrical configurations (also referred to herein as “TheCOS”) is also disclosed. Aside from utility in experimental sciences and biology the technology has translation potential and application. The cell model is able to help form multi-layered sheets, spheres, and structure with other shapes. Its dynamic feature also enables tissue constructs having adjustable size, cell contacts, cell distributions and inner structures. Thus, TheCOS can be used to form complex cellular structures as needed, such as bringing together complex combinations of cells to create a synthetic organoid or to promote the growth of a complex multicellular tissue-like structure. In some aspects, as shown in the Example, the cell model is an in vitro model for studying the effect of small molecules or genomic modification of cytotoxic cells on cell cytotoxicity.

In some aspects, a method of forming complex cellular structures, such as that of TheCOS is disclosed. The method comprises: preparing and assembling an upper mold and a lower mold with desired shape and size; forming an assembled mold structure; preparing a hydrogel composition containing desired components; and preparing a cell suspension. The method further comprises add a portion of the hydrogel composition to the assembled mold structure, and, after cooling, remove the upper mold. The desired amount of the cell suspension is then added to the lower mold, The upper mold and the lower mold are reassembled, and the cell suspension is allowed to gel. Once the cell suspension is gelled, the upper mold is removed. The steps of adding cell suspension to the lower mold and gelling the suspension is repeated to prepare a desired layered cell strata. Finally, a portion of the hydrogel composition is added into the lower mold to seal the cell strata.

In another aspect, a method of preparing a synthetic tissue-like spheroid using a water-in-oil emulsion is disclosed. The method comprises: prepare a cell suspension; adding a portion of the cell suspension to mineral oil; extracting gelated microbeads from mineral oil with warmed DPBS solution; performing continuous dilution to isolate single microbeads; and co-culturing isolated microbeads with target cells in incubator to produce spheroid. The method further comprises sealing the spheroid by adding agarose hydrogel. In certain implementations, the composition of cell suspension to mineral oil is mixed vigorously while warmed.

### EXAMPLES

### Example 1. Establishing TheCOS, a Dynamically Adjustable 3D Culturing System

Relative positions and dynamic interactions between different groups of cells carry profound biological significance. Highly organized segmentations inside tissues and organs accommodate and regulate complex biological processes, such as the maturation and education of immune cells in thymus and lymph nodes. Meanwhile, active translocations via circulation routes or migration mechanisms are vital for multiple cell subsets to execute their functions, such as the tumor infiltration by cytolytic lymphocytes. To reproduce the intercellular interactions happening in physiological conditions, people need to model a complex multicellular structure, and most importantly, bring dynamic spatial control into it.

For dynamically adjustable 3D culturing, there are three other technologies that can achieve similar outcomes: 1) Chemo-responsive hydrogel; 2) UV-responsive hydrogel; and 3) Microfluidics system. Similar to TheCOS, both 1) and 2) also utilize responsive hydrogels to change multicellular structure and can translocate cells through designated paths. Unlike our technology, however, 1) relies on enzymes (usually proteases) or other reactive chemicals to depolymerize the backbones of hydrogel, while 2) projects spatially modulated UV light to degrade hydrogels at selected sites.

Due to the rate limit of depolymerization, chemo-responsive hydrogels all share slowness. The attempts to speed reaction by increasing enzymes or reactive chemicals are hindered by increased cost and cytotoxicity. Similarly, degradation rate of UV-responsive hydrogels positively correlates with UV-light intensity, thus also limited by UV-induced phototoxicity. Moreover, 2) requires special lithographic optics to project UV light to selected areas, making this technology significantly expensive. Microfluidics systems utilize delicate micro-channels and chambers to manipulate behavior of fluids and small particles. Carried by well-designed flow, cells can be moved rapidly and precisely with minimum mechanical stress. Although potentially sophisticated, these devices (usually being built on chips) are highly customized for unique experimental purposes and suffer from high cost and lack of flexibility.

Thus, TheCOS, an in vitro model, was developed. TheCOS can be used to study effect of certain small molecules on NK cell cytotoxicity based on the general strategy of establishing close intercellular contacts between designated cell groups in multiple three-dimensional geometrical configurations. It can rapidly move cells in a controllable, mechanically gentle manner and is significantly cheaper compared to other technology. TheCOS can also be used for creating an experimental model that studies cell behaviors in cell aggregates as well as constructing multi-layered sheets or spheres in tissue or organoid engineering.

Responsive hydrogels consisting of temperature-sensitive polymers are utilized to segregate and aggregate cells in a spatially and temporally controlled manner. These responsive hydrogels undergo gel-solution phase transition following the tuning of environment temperature. This transition greatly changes mechanical properties of and triggers spatial collapse at the designated part of a multicellular structure. During the collapse, cells will experience rapid yet mechanically gentle translocation.

Compared to other similar technologies, TheCOS only relies on global temperature control, and thus makes itself non-contact, ultra-low cytotoxic, equipment-light, low-cost and compatible with common real-time imaging platforms.

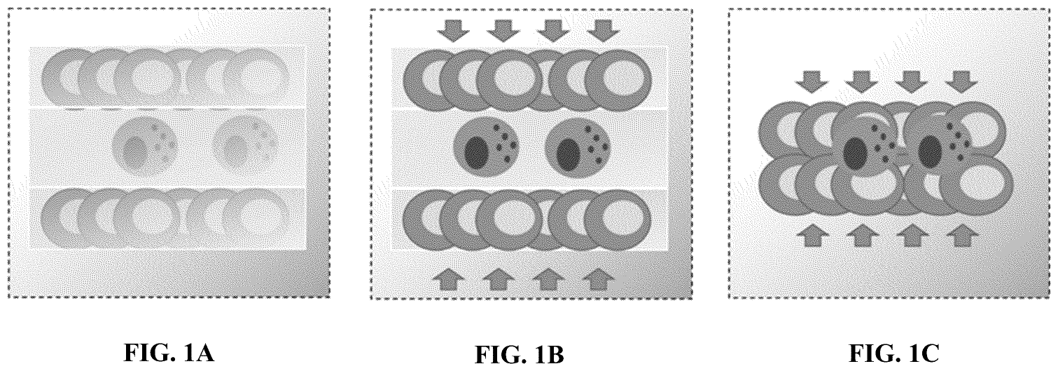

As one specific case (FIGS. 1A-1C), cells of interest are embedded in responsive hydrogels consisting of temperature-sensitive polymers. These cells may be a single type of cell (such as, NK92 cell, Jurkat cell, HEK293T cell, and YTS cell), or a mixture of different cells. Under a temperature stabilizing gel-state, cell-embedding responsive hydrogels are parallelly layered together to form a strata structure. Around it, a cell-free, non-responsive hydrogel jacket is built to fully surround the strata (FIG. 1A). When temperature is altered to allow gel-solution transition, embedded cells are unleashed from the entanglement by extracellular matrices and can move freely in polymer solution (FIG. 1B). Meanwhile, compressed by the internal forces of non-responsive hydrogel jacket, the central spaces previously occupied by cell strata gradually collapse and all cells are extruded to a single sheet, where cells are densely packed but still preserve the original pattern of layered distribution (FIG. 1C).

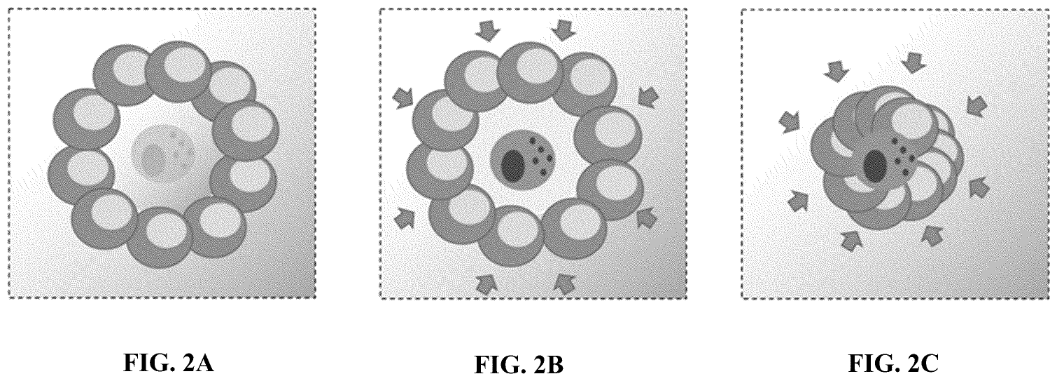

As another specific case (FIGS. 2A-2C), a single cytolytic cell is embedded in a responsive hydrogel microbead consisting of temperature-sensitive polymers. Under a temperature maintaining gel-state, a spheroid of target cells is formed around the microbead and then a cell-free, non-responsive hydrogel jacket is built outside to fully surround the spheroid (FIG. 2A). When temperature is altered to allow gel-solution transition, the central microbead collapses and the spheroid is compressed by the internal forces of non-responsive hydrogel jacket (FIG. 2B). Target cells are pushed toward center, where they meet the unleashed cytolytic cell and form close intercellular contacts with it (FIG. 2C).

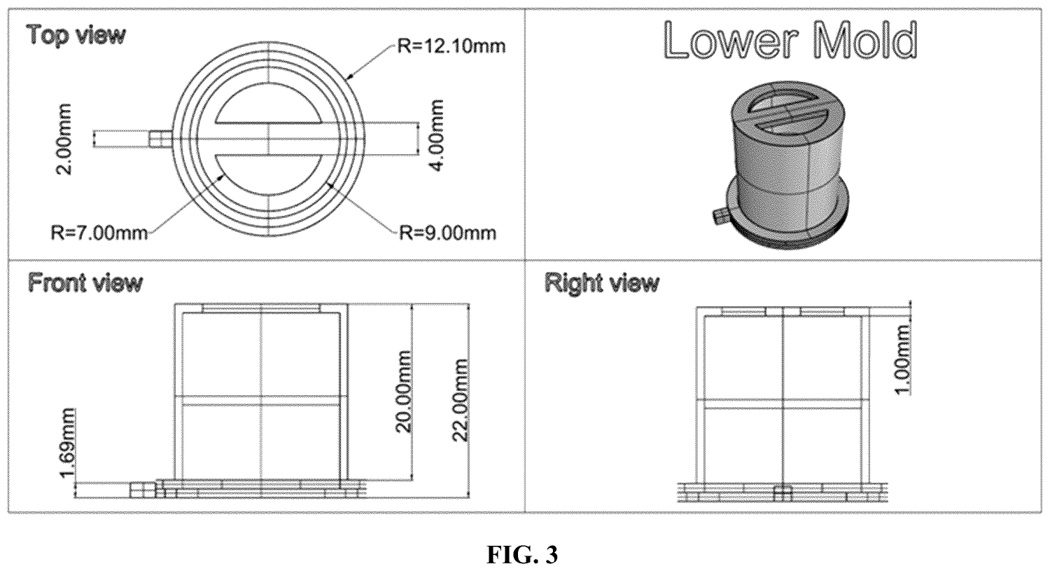

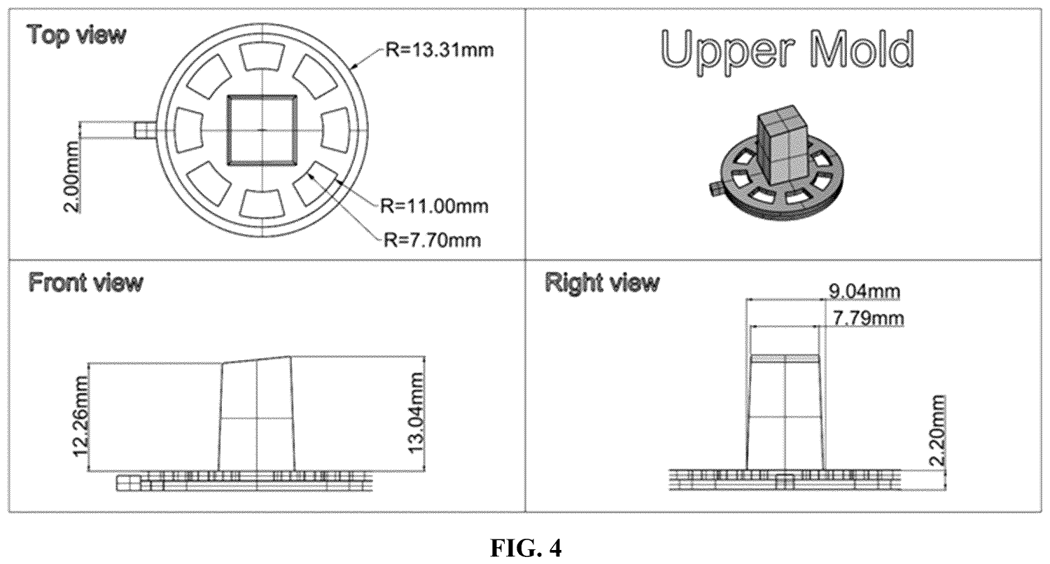

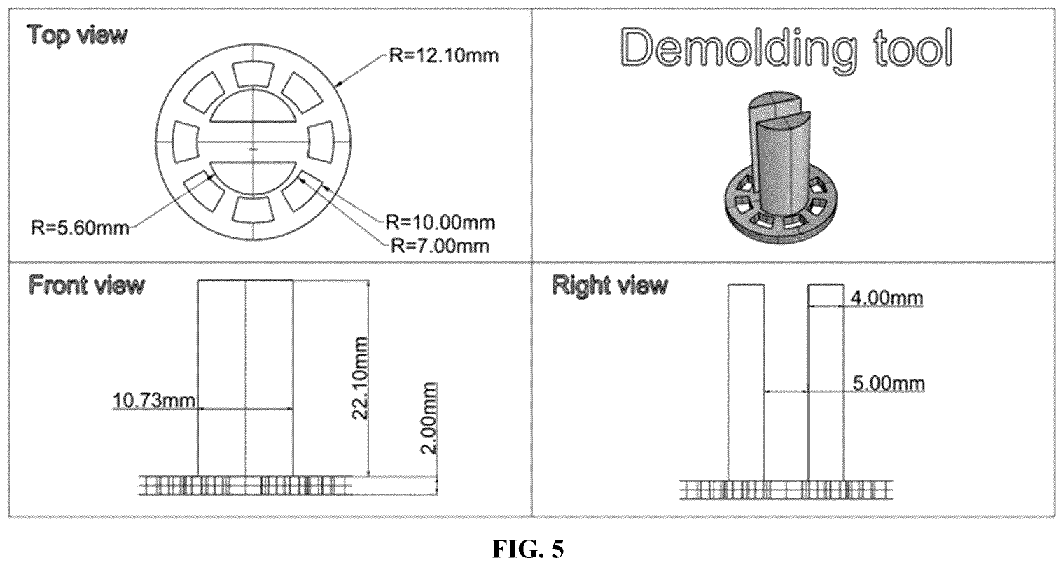

Self-designed and produced equipment used in TheCOS are: lower mold (FIG. 3), upper mold (FIG. 4), and demolding tool (FIG. 5).

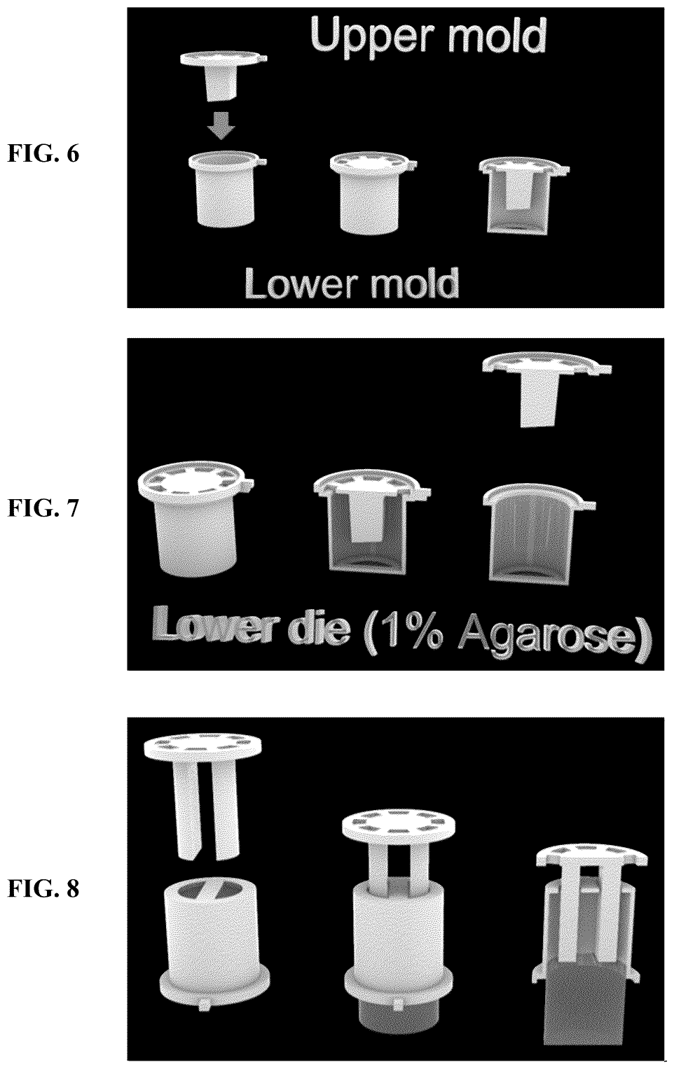

Cell strata described above are prepared by sequential extrusion. Briefly, the steps are:
1) Assemble upper and lower molds together. (FIG. 6)
2) Pour 45° C. 1% agarose hydrogel into the mold assembly and cool it under room temperature for 20 min to make the lower die of cell strata. After sufficient cooling, carefully remove upper mold to expose the lower die. (FIG. 7)
3) Prepare cell suspension with desired concentration in 1% PNIPAM solution. Maintaining environment temperature below the phase transition temperature of PNIPAM (Tₘ˜ 32° C.).
4) Add certain volume of cell suspension into the bottom of lower die. The volume should be calculated from the desired thickness of cell stratum (see table below). Insert and gently extrude the upper mold toward the lower die until it reaches to the bottom.

TABLE 2

| Desired thickness of cell stratum | Volume of cell suspension |
|---|---|
| 50 μm | 14 μl |
| 100 μm | 28 μl |
| 150 μm | 42 μl |
| 200 μm | 56 μl |

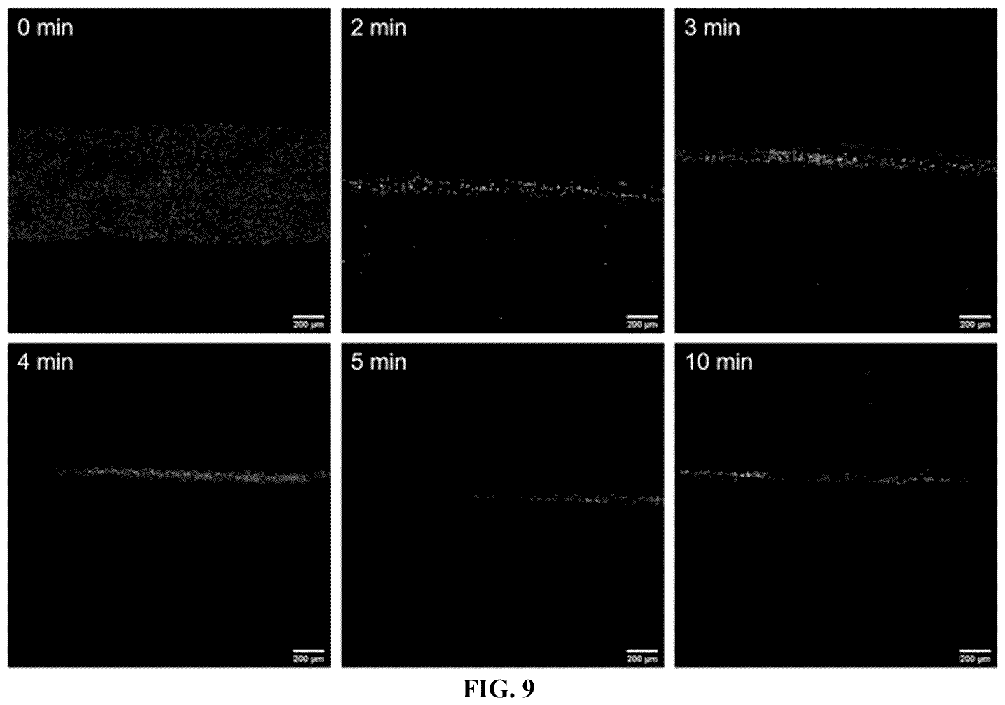

5) Place the assembly on isothermal plate (37˜40° C.) for 5 min to complete the gelation of cell suspension. After sufficient warming, carefully remove upper mold to expose the lower die.
6) Repeat step 4 and 5 to build new cell stratum on top of old ones.
7) Finally, pour 45° C. 1% agarose hydrogel into the lower die to fill it up and seal cell strata. Cool it on isothermal plate (37˜40° C.) for 30 min.
8) Carefully remove the gel assembly (agarose+PNIPAM) from the lower mold using demolding tool. Always keep gel assembly contacting with isothermal plate during the operation (FIG. 8).
9) To maintain the structure of cell strata, keep the gel assembly in a warm (>Tm) and humid environment.
10) To trigger the collapse of cell strata, just cool the gel assembly down below transition temperature for 5˜10 min (FIG. 9).

Spheroids described above are prepared by water-in-oil emulsion. Briefly, the steps are:
1) Prepare cell suspension with desired concentration in 1% PNIPAM solution.
2) Pre-warm cell suspension and mineral oil to 28° C.
3) Add 100 ul of cell suspension into 2 ml of mineral oil. Using the highest speed, vortex the water-in-oil mixture vigorously for 30 second. After vortexing, immediately immerse emulsion vessel into 37° C. water bath and keep shaking it vigorously for 3 min.
4) After being lifted from water bath, emulsion vessel needs to be wiped off and placed on isothermal plate (37˜40° C.) immediately.
5) Extract gelated microbeads from mineral oil with pre-warmed (37˜40° C.) DPBS (Dulbecco's phosphate-buffered saline) solution.
6) Count the concentration of microbeads in warm DPBS solution and perform continuous dilution to get single microbeads in 96-well plate. The whole procedure needs to be done on isothermal plate.
7) Co-culture isolated microbeads with targets cells in incubator for 72 hours to grow spheroid.
8) Seal spheroid by adding equal volume of 45° C. 2% agarose hydrogel in to each 96-well. Cool it on isothermal plate (37˜40° C.) for 30 min.
9) To maintain the structure of spheroid, keep it in a warm (>Tm) and humid environment.
10) To trigger the collapse of spheroid, just cool it down under room temperature for 5˜10 min.

To assess tumor cell and bystander killing, TheCOS comprises NK cell lines, tumor cell targets, and NK-resistant tumor cell bystanders. LAMP1-mApple-pHluorin-expressing NK cell lines enable visualization of granules (mApple) and degranulation events (pHluorin transition). mTurquoise2 mark susceptible tumor cells, and Citrine mark bystander cells. SYTOX deep red is included in the hydrogel to identify the nuclei of dying cells. A cell positive for Turquoise and SYTOX fluorescence denote a dying tumor cell, whereas a cell positive for Citrine and SYTOX indicate a dying bystander cell. 1×10⁵ target tumor cells and 1×10⁵ NK cells (for an initial 1:1 tumor to effector ratio)+/−1×10⁶ bystander cells are deposited into separate thermally responsive hydrogel layers and then encased in thermally stable hydrogel. This sandwich is then deposited the into our imaging chamber. Prewarmed media is added to collapse the hydrogel layers.

FIGS. 8A and 8B depict one of the original “sandwiches” showing bystander killing (labeled with cell membrane and vital cell dyes, which are alternatives to fluorescent protein-based approaches). In NK/tumor target cell only experiments, a successful candidate increases MGD and radial degranulation and allows an NK cell to kill multiple target cells at the same time. When bystander cells are included, dispersed conditions also result in collateral killing of non-triggering cells. CIL-D-treated or HkRP3-deleted NK cells are positive dispersion controls. Vehicle-treated or vector only NK cells are negative controls.

More complex tumor microenvironments are modeled by including surrogates for a triggering tumor cell (e.g. 721.221 cell), an escaping tumor cell (e.g. NK cell-resistant Raji cell), a tumor stromal cell (e.g. HS-5 human stroma), and a suppressive tumor resident immune cell (e.g. THP-1 macrophage (72)) in the hydrogel stack. Each cell is transduced with one of several fluorescent proteins to allow simultaneous detection (for example, mTurquoise2, Citrine, and LSSmOrange) or labeled using cell dyes. Each cell type will be suspended in its own thermal-responsive hydrogel, layered, encased, and collapsed as above.

### Example 2. Methods Related to the Identification of Genomic Modification or Small Molecules that can Sustain Lytic Granule Dispersion

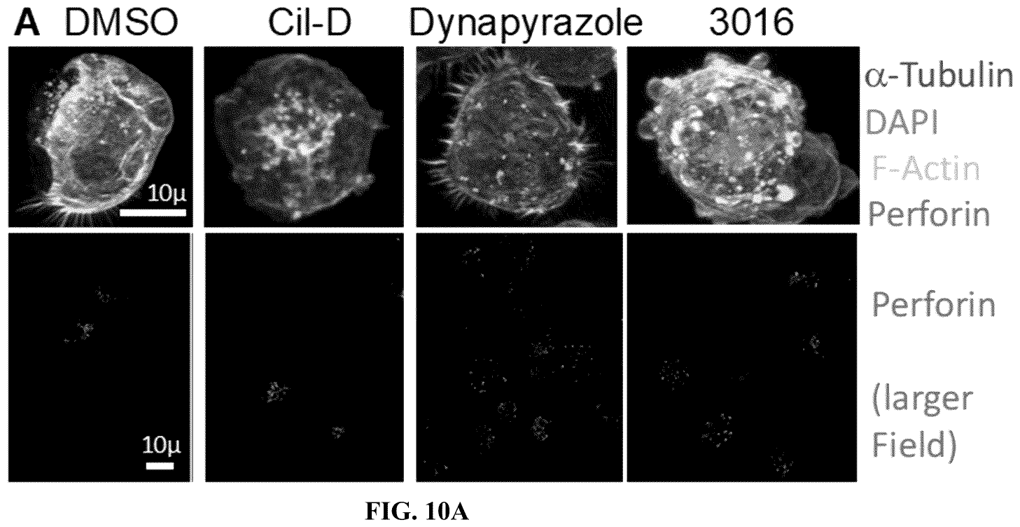

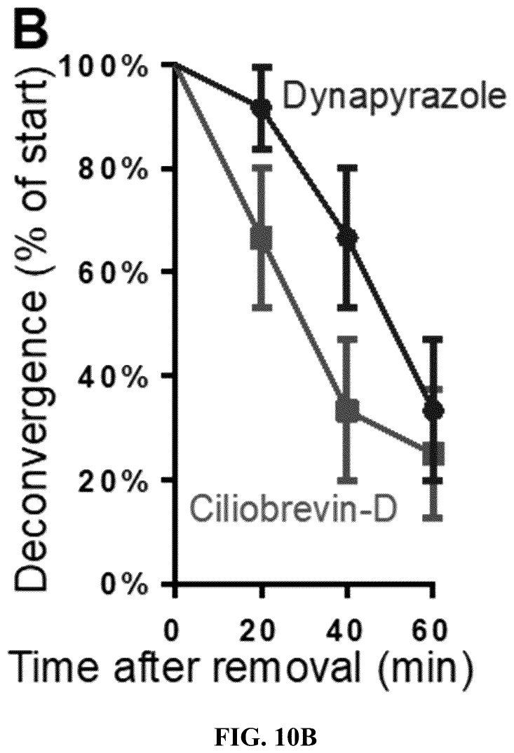

Dynein inhibitor Ciliobrevin D (CIL-D, 2-(7-chloro-4-oxo-3,4-dihydroquinazolin-2 (1H)-ylidene)-3-(2,4-dichlorophenyl)-3-oxopropanenitrile; CAS No: 1370554-01-0) and LFA-1 blocking antibodies have been previously used to inhibit lytic granule convergence and enable non-directional degranulation. Both approaches promoted killing of bystander cells in UGATm (Ultrasound-Guided Acoustic-Trap microscopy) and other single cell assays. This work served as proof-of-concept that influencing positional degranulation can be used to harness the NK cell lytic machinery. However, in the case of CIL-D, inhibition of convergence was quickly reversible (FIG. 10B) and required high concentrations in culture media. Like CIL-D, dynapyrazole-A and Compound 3016 were also highly effective in preventing lytic granule convergence (FIG. 10A). However, the effect of dynapyrazole-A in inhibiting convergence is more sustained than CIL-D (FIG. 10B).

If cytotoxic therapy cells were treated in vitro and then infused into a patient, CIL-D effects would dissipate prior to them reaching a tumor. LFA-1, on the other hand, is a major NK cell adhesion receptor. Its binding by ICAM during adhesion-mediated extravasation promotes convergence, priming the cell for directed killing outside of the circulation. While the effect of blocking antibodies can be durable enough for clinical use, it is likely that blocking LFA-1 will also inhibit extravasation, precluding therapeutic cell delivery to the diseased site of interest. While these experiments prove that convergence can be influenced experimentally, new tools are clearly needed.

Table 1 lists the candidate genes/proteins and molecules along with methods for their testing in single cell and complex cell environments to determine whether granule dispersion may be sustained without disrupting other critical NK cell functions. These candidates impact viability, proliferation, degranulation, actin accumulation/function, and polarization.

TABLE 1

| Genes/Proteins | Small Molecules |
|---|---|
| GOLGA1: Golgin-97 | Centrinone: 2-[[2-Fluoro-4-[[(2-fluoro-3- |
| GOLGA4: G-247, | nitrophenyl)methyl]sulfonyl]phenyl]thio]-5-methoxy- |
| Golgin-247 | N-(5-methyl-1H-pyrazol-3-yl)-6-(4-morpholinyl)-4- |
| GCC2: GCC2 | pyrimidinamine |
| GCC1: GCC1 | CAS No: 1798871-30-3 |
| SEPT7: Septin-7 | Concentration: 5 nM |
| SEPT2: Septin-2 | CFI400945: Spiro[cyclopropane-1,3′-[3H]indol]- |
| F8A1: HAP40 | 2′(1′H)-one, 2-[3-[(E)-2-[4-[[(2R,6S)-2,6-dimethyl-4- |
| ARFGEP1: BIG1 | morpholinyl]methyl]phenyl]ethenyl]-1H-indazol-6-yl]- |
| CENATAC: CCDC84 | 5′-methoxy-, (1R,2S)-, (2E)-2-butenedioate (1:1) |
| HkRP3 | CAS No: 1338806-73-7 |
| CLIP170 | Concentration: 50 nM |
| VASP | Dynapyrazole A: 2-[1-(4-Chlorophenyl)cyclopropyl]- |
| PYK2 | 4,5-dihydro-7-iodo-5-oxopyrazolo[1,5-a]quinazoline- |
| RABGGTA | 3-carbonitrile |
| ACTB | CAS No: 2226517-75-3 |
| | Concentration: 5 μM |
| | Compound 3016: TDI 3016 aminopyrazole- |
| | containing dynapyrazole derivative |
| | LPA: 1-Oleoyl lysophosphatidic acid sodium salt |
| | CAS No: 325465-93-8 |
| | Concentration: 20 μM |
| | Compound 5814: N-{1-[(4- |
| | chlorophenyl)methyl]piperidin-4-yl}prop-2-enamide |
| | Concentration: 5 μM |
| | Compound 7779: 1-(prop-2-enamido)-N-[(thiophen- |
| | 2-yl)methyl]cyclohexane-1-carboxamide |
| | Concentration: 5 μM |
| | Compound 3977: 1-[3-(4-fluorophenyl)prop-2- |
| | enoyl]-4-hydroxypyrrolidine-2-carboxylic acid |
| | Concentration: 5 μM |

Each candidate according to its impact on three critical synaptic functions: 1) degranulation; 2) polarization, and 3) actin accumulation/function. The candidate inhibitors are used to pretreat NK cells as above, while the candidate genes are removed or reduced via CRISPR editing or siRNA.

CD107a (also known as LAMP-1) is the standard to measure the process of degranulation. The more CD107a is expressed on the surface of the cell after stimulation, the more granules have been released. This is limited to the immunological synapse, in part secondary to the convergence process. CD107a upregulation is measured via flow cytometry after PMA/lonomycin activation and can be measured following target cell conjugation. The former bypasses receptor ligation and signal transduction and therefore reveals more about terminal degranulation processes. The latter requires synapse formation and engagement of receptors for downstream activation signaling. Differences in results between these two approaches (target cell conjugation versus PMA/lonomycin) map the candidate's impact to early (synapse formation and signaling) or late (mechanics of) degranulation.

To assess polarization, an MTOC-to-activating surface assay is used. NK cells are incubated on a glass surface coated with anti-NKp30/anti-CD18 or anti-CD28/anti-CD18, and then fixed and stained with pericentrin to identify the MTOC. The stained cells' MTOC distance from the slide are measured following a time that allows for maximal polarization to occur. Since MTOC polarization is required for directed secretion, candidates that regulate this process represent critical activation-induced lytic synapse functions and potential opportunities to influence therapeutic function. Since actin organization precedes this and is required for MTOC polarization, proteins that block the latter without affecting the former will be especially relevant.

To assess actin accumulation, NK cells are incubated on an anti-NKp30/anti-CD18-coated or anti-CD28/anti-CD18 glass surface (or to confirm, with target cells), stained with phalloidin, and the thickness and extent of actin accumulation is measured. If actin accumulation is abnormal, the nature of the disruption is then defined.

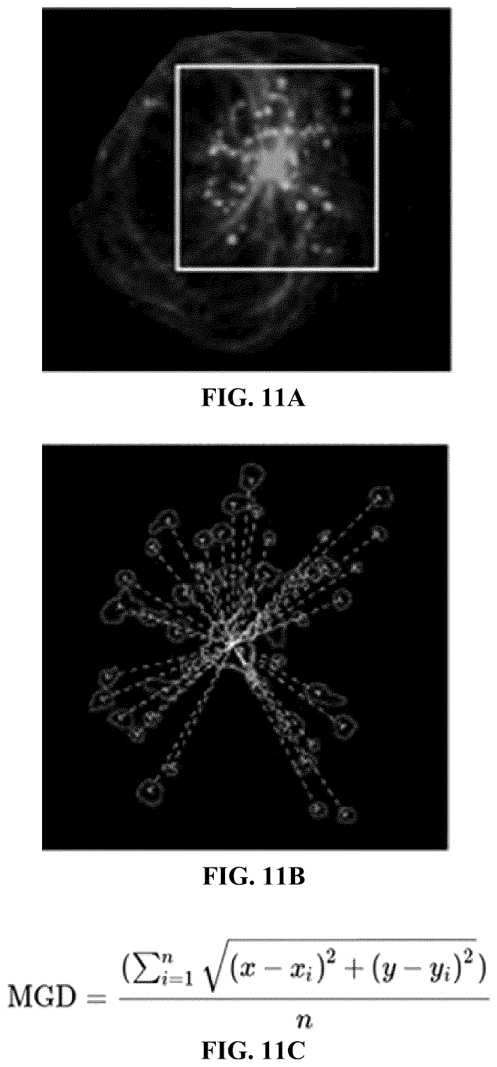

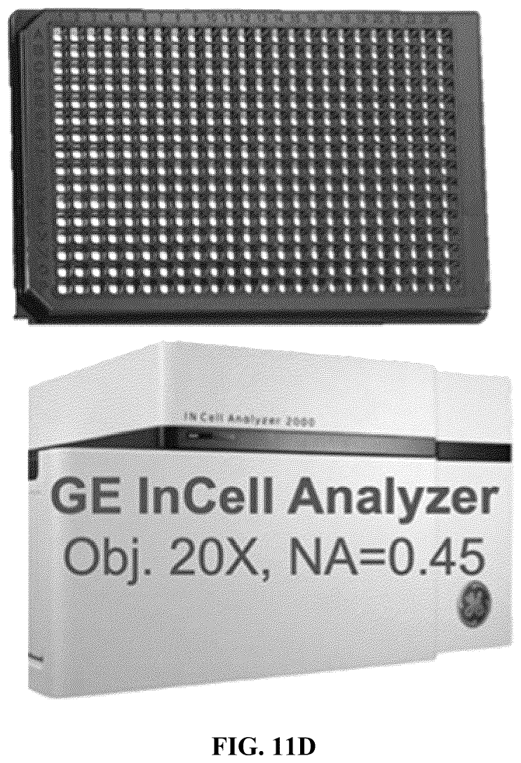

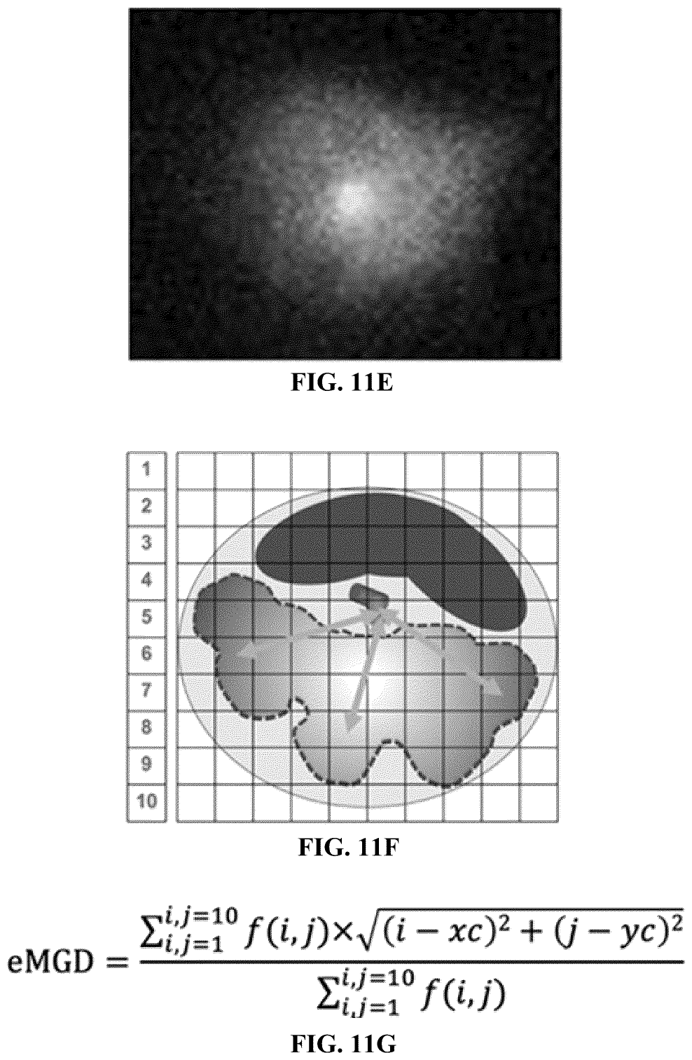

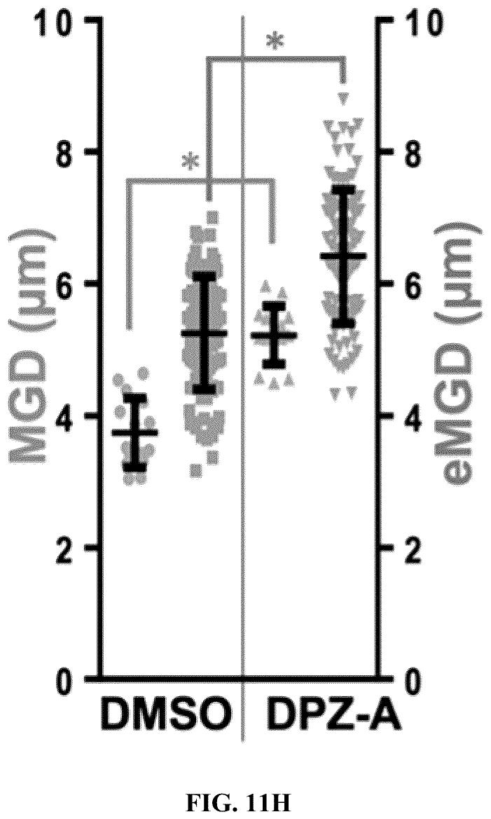

To identify the best candidates to control lytic granule positioning, a high throughput screen (HTS) for activation-induced lytic granule convergence was developed. This is a novel method that utilizes a simple shape-based approach (a grid based on the a-tubulin defined NK cell footprint and Euclidean distance from the MTOC) to generate an estimated mean granule distance (eMGD) from the MTOC (FIGS. 11A-11C). This approach allows us to quickly screen large numbers of molecular candidates. MGD will be measured in 3D in fixed NK cells activated on an anti-NKp30/anti-CD18 or anti-CD28/anti-CD18 coated glass surface over a range of activation times, conditions, and NK cell types. Cells will be stained to identify the MTOC (for example, pericentrin) and lytic granules (mature perforin). The measured MGD is an indicator of convergence.

Briefly, glass bottom 384-well plates (such as in FIG. 11D) coated with anti-CD18 and anti-NKp30 are loaded with an optimized number (5×10³ in 15 μl) of YTS or NK92 cells for 30 min at 37° C. prior to fixation, permeabilization, and staining with anti-a-tubulin and anti-perforin. Liquids are handled by a Beckman Coulter Echo and cells are imaged in a GE InCell Analyzer using a 20×0.45NA objective. While this allows low-resolution detection of lytic granules and MTOC in a large number of samples, individual granules and mean granule distance from the MTOC (MGD) cannot be measured. However, a Euclidean method for assessing granule localization relative to a total cell, tubulin-defined footprint can be used to estimate MGD (FIG. 11F). eMGD is then calculated using a Matlab script and the formula of FIG. 11G. The script pixelates the image, retrieves the MTOC position from the a-tubulin channel (xc, yc), and then traverses all pixels in the perforin channel, first calculating the intensity of each pixel f(i,j) and then defining its distance from the MTOC. Pixels are then summed to generate eMGD.

Compounds are dispensed by the Echo liquid handler into anti-CD18 and anti-NKp30 coated wells across three concentrations (working range of known inhibitors) prior to adding NK cells and then activating, staining, and imaging via the method described above.

Reversibility time for convergence is determined by activating small molecule inhibitor pre-treated NK cells on anti-NKp30/anti-CD18-coated or anti-CD28/anti-CD18 glass surfaces in the absence of the compound of interest. In short-term live-cell experiments, cells are labeled with lysotracker and fluorescent protein-conjugated tubulin; for longer term experiments, the more durable lytic granule label, LAMP1-mApple, is used. The efficiency of non-directional degranulation will be measured with the previously disclosed LAMP1-pHluorin cell line, which marks the lytic granule pH change that accompanies degranulation. Indicator-expressing NK cells will be pretreated with candidate compound or be deleted of the gene of interest, activated on anti-NKp30/anti-CD18-coated or anti-CD28/anti-CD18 glass, and imaged live through 3-dimensions at 1-minute intervals.

### Identifying Candidate Genes:

Well-established CRISPR-Cas9 gene interruption methods are used in NK cell lines and gene knockdown in ex vivo NK cells to identify genes/proteins that promote, enable, and/or maintain granule convergence. The CRISPR-Cas9 gene interruption methods involve stable doxycycline-inducible Cas9-expressing YTS and NK92 cells into which the lentiviral CRISPR pools will be introduced. Lentiviral transfection is performed in 96-well plates. An optimal number of transfected cells are then be transferred into 396 well plates for the convergence screening as described above for identifying candidate inhibitors.

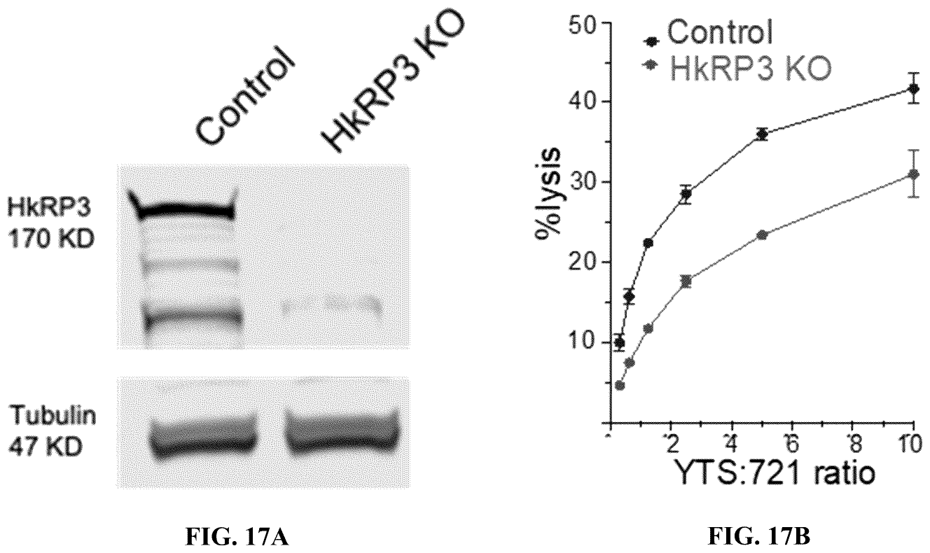

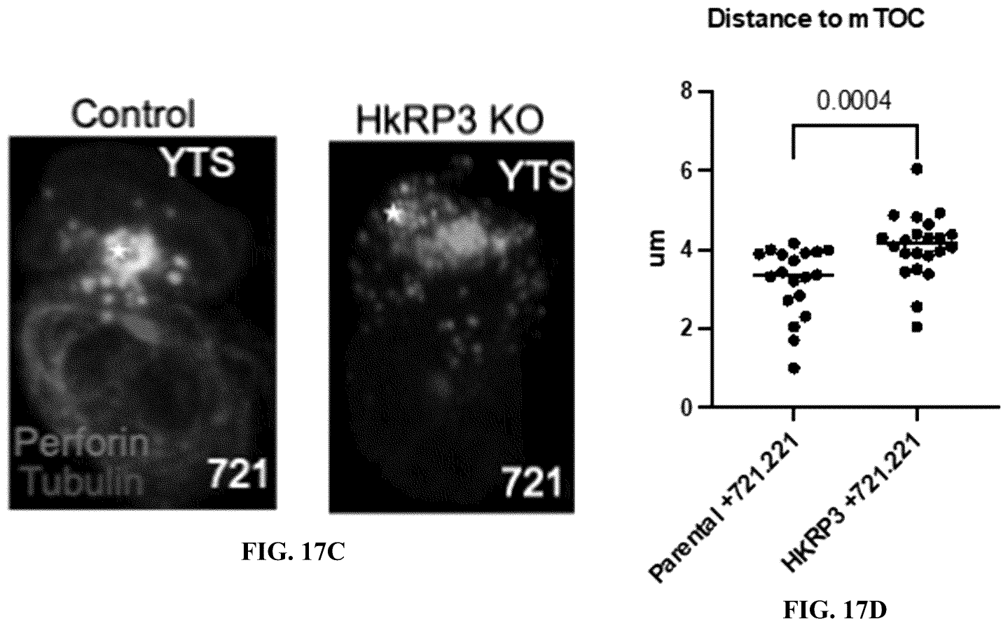

The target genes/proteins are listed in Table 1 and include the hook-related microtubule binding protein HkRP3, the microtubule plus end tracking protein CLIP170, the actin regulatory protein VASP, and the activation signaling protein PYK2. CRISPR-Cas9 editing of HkRP3 in YTS NK cells promotes granule dispersion and alters activation-induced granule positioning, although it also reduces cytotoxic efficiency in short-term standard killing assays (FIGS. 17A-17D).

### Example 3. Small Molecules for Inducing Lytic Granule Dispersion

### 1. Dynapyrazole A

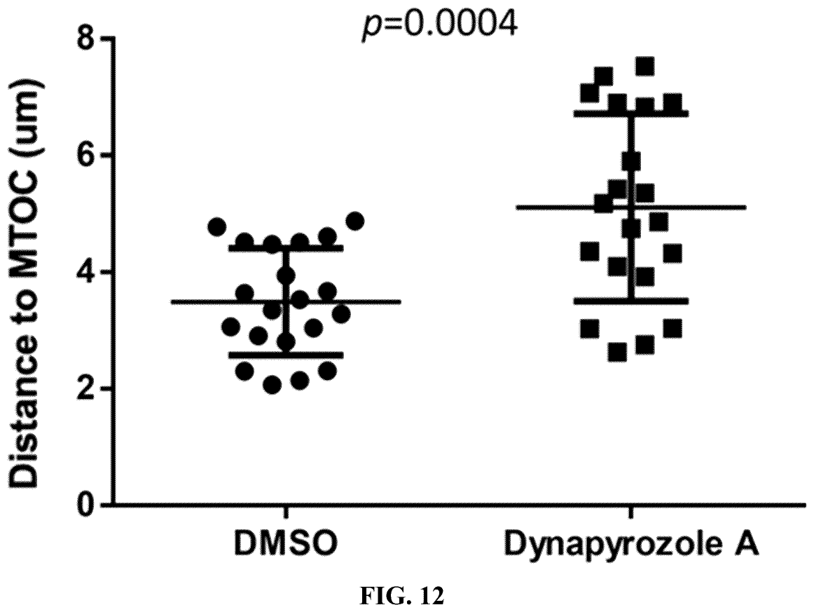

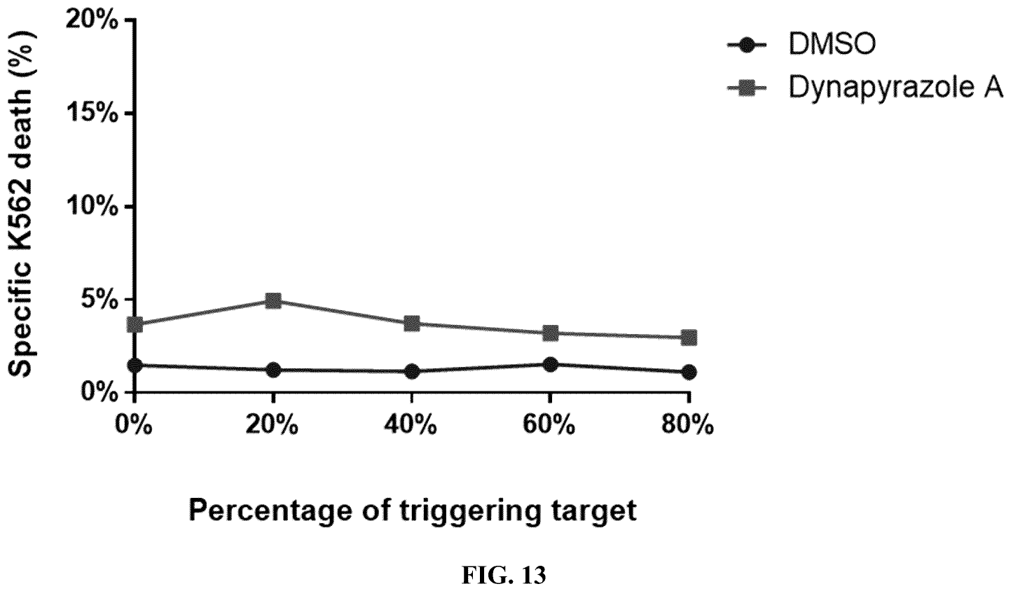

YTS cells are treated with 5 μM of Dynapyrazole A for 30 minutes, then washed twice with culture media. Then cells are stimulated either with 721.221 target cells, K562 target cells, or with an activating surface (coverslip coated with anti CD18 and/or anti CD28). Dynapyrazole A induces lytic granule dispersion in YTS cells (FIG. 12). Dynapyrazole A-pretreated YTS cells kill bystander K562 cells (FIG. 13).

### 2. LPA

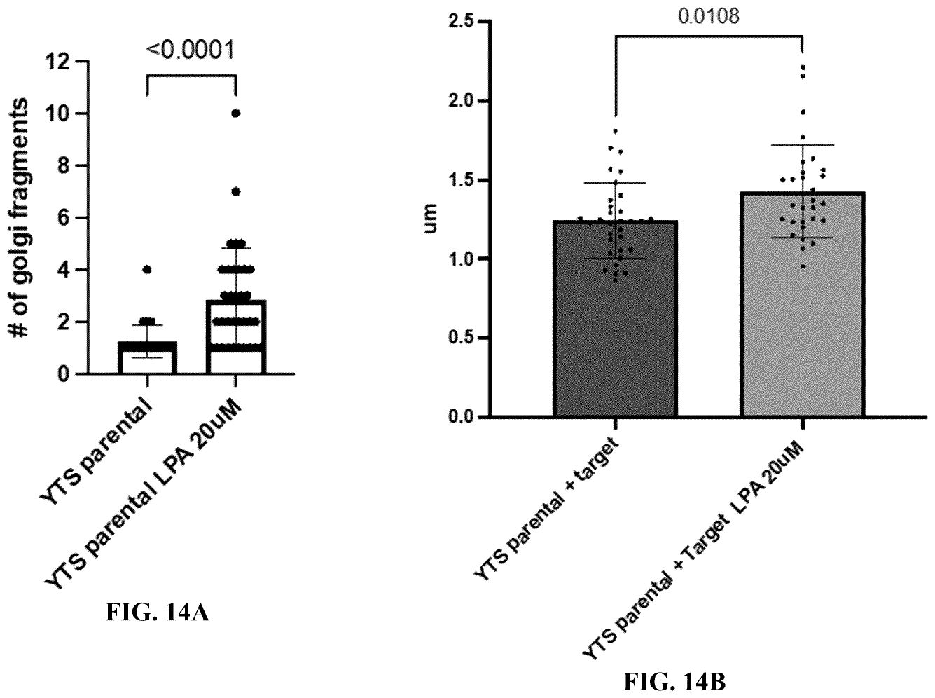

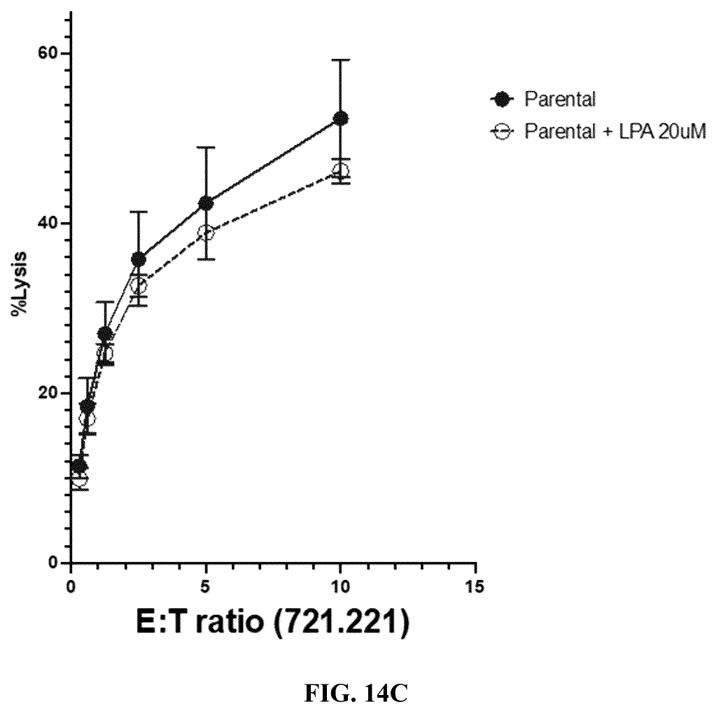

One way to induce the Golgi outpost formation through elongation and fission, is using LPA, which is predicted to activate a pathway dependent on transforming protein RhoA and effectively induce the formation of Golgi Outpost. YTS cells were stimulated with a 20 μM LPA for two hours and then evaluate Golgi fragmentation and granule dispersion (FIGS. 14A and 14B, respectively). The cells were also tested for Cr-51 (FIG. 14C). The treatment with LPA blocks convergence, enabling widespread granule dispersion that is associated with an increase in Golgi fragments. This dispersion was thus increased in quantity and spread on the GCC2 KO cells.

### 3. CFI-400945 and Centrinone

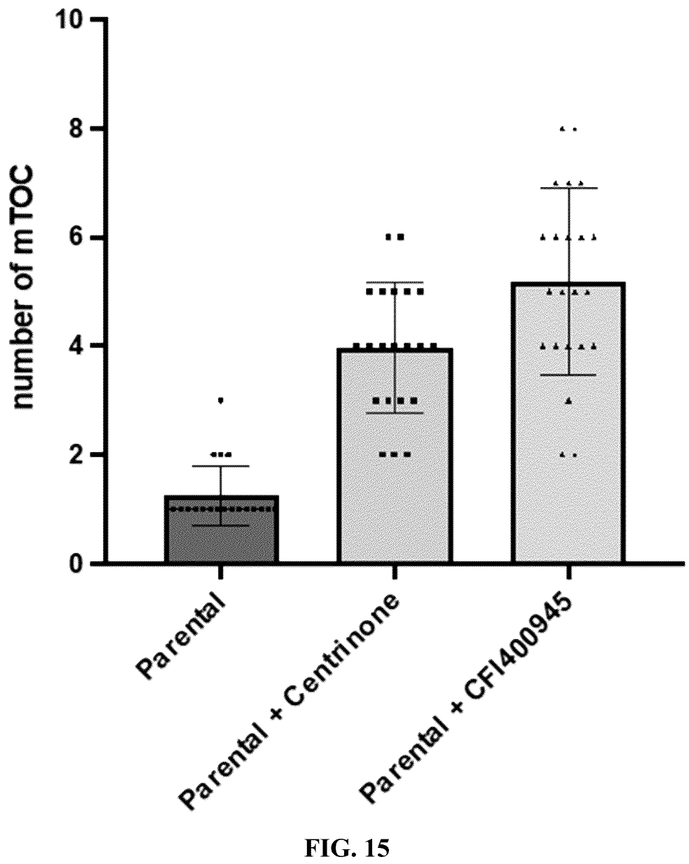

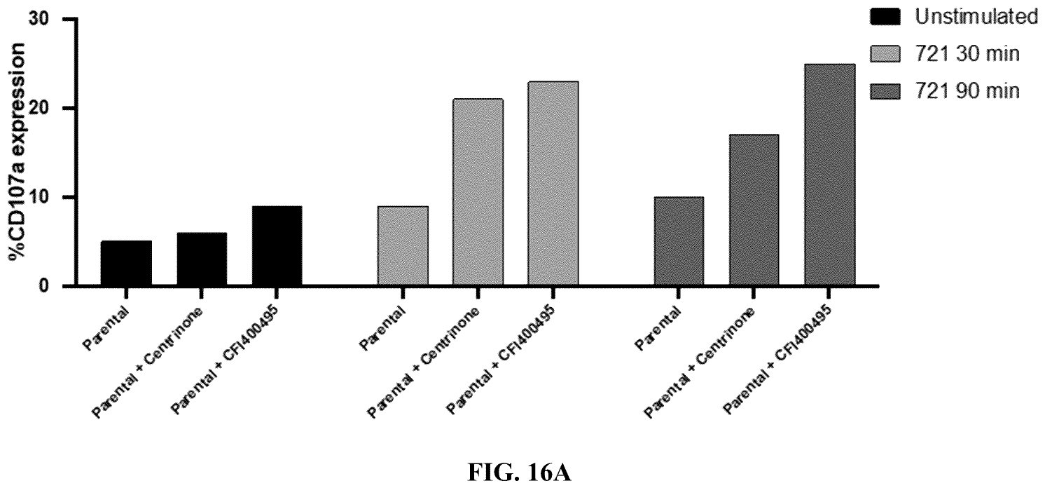

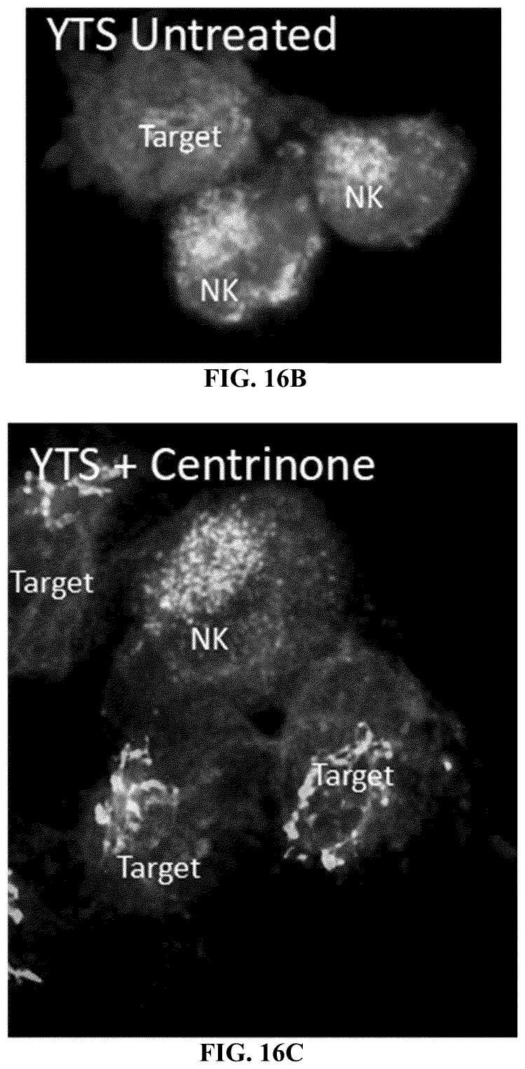

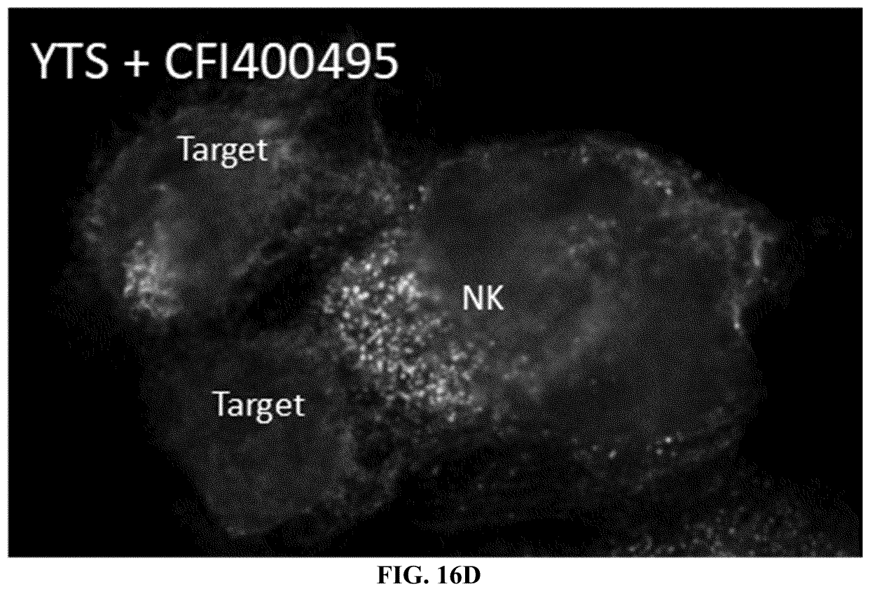

Centrinone and CFI400495 are high affinity and selective PLK4 inhibitors that induce depletion of the centrosome and cell cycle arrest. YTS cells were treated for 5 days with Centrinone 5 nM or CFI400495 50 nM. Cells treated with either inhibitor showed an increase number of mTOC, measured as the number of alpha Tubulin hyper densities (FIG. 15). Treated cells also showed an increase degranulation after conjugation with targets cells 721.221 (FIGS. 16A-16D). For this experiment, 5×10⁵ cells/ml YTS cells were incubated during 5 days Centrinone 5 nM or CFI400495 50 nM, every 24 hours the media were replaced with fresh media containing similar amounts of the drug. On day 5, cells were assessed by microscopy for gamma-tubulin or alpha-tubulin.

4. N-{1-[(4-chlorophenyl)methyl]piperidin-4-yl}prop-2-enamide (Compound 5814)

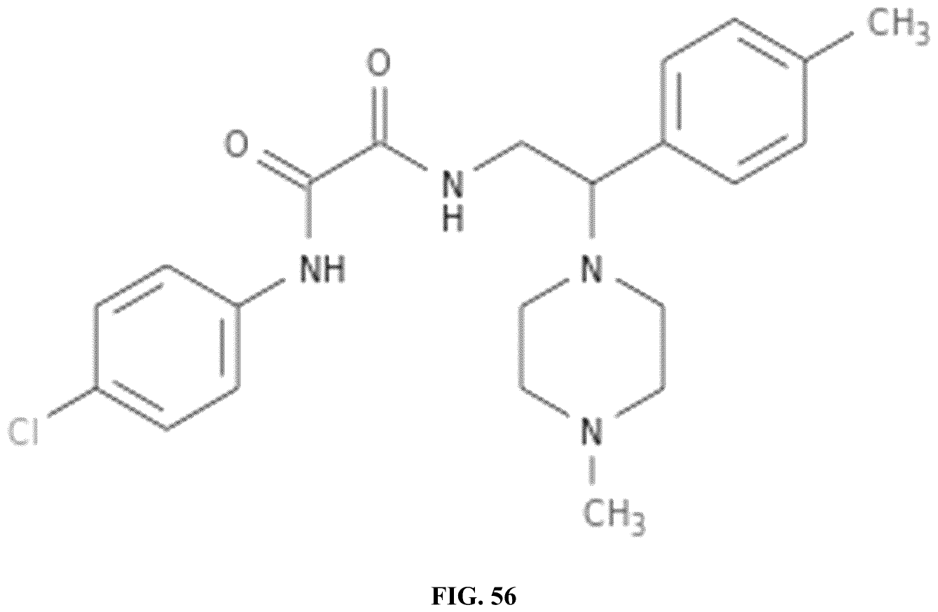

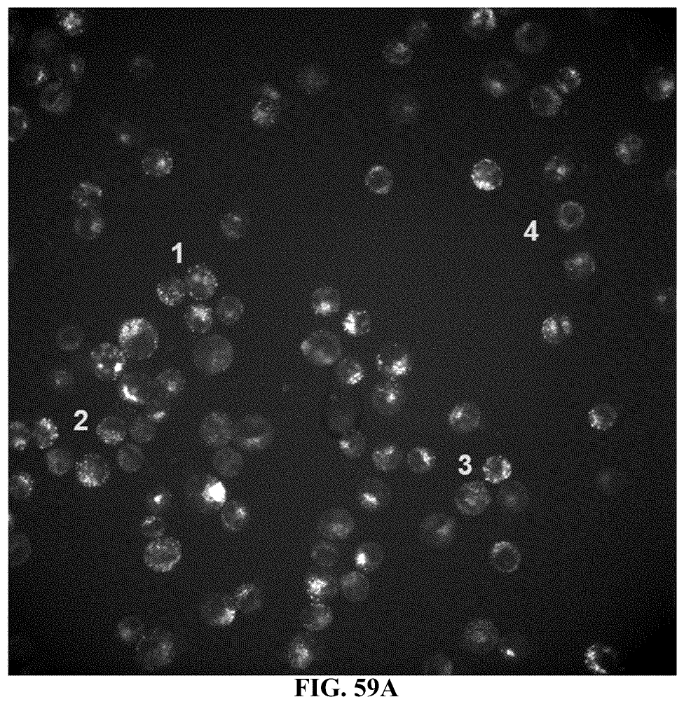

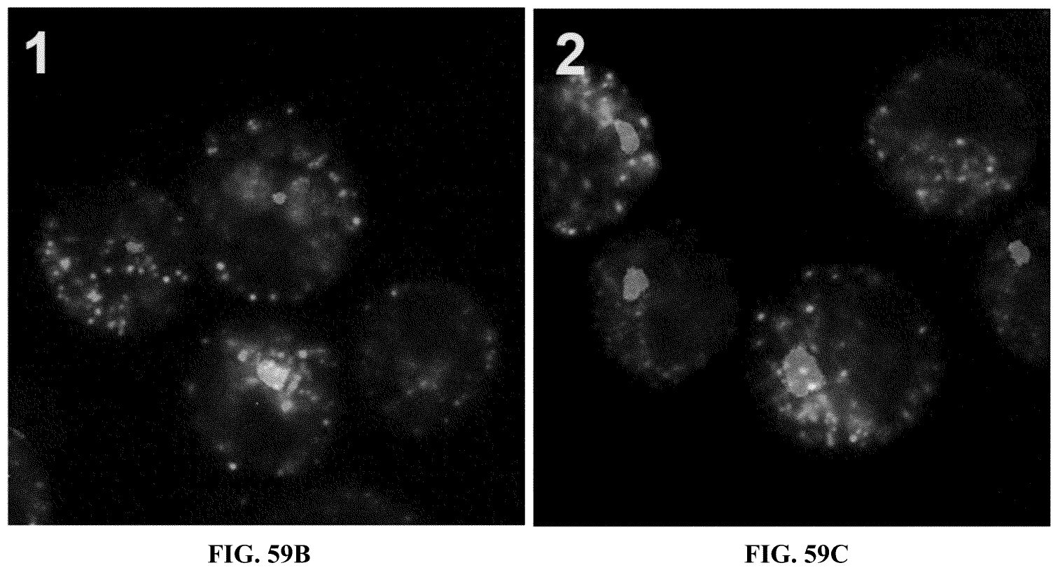

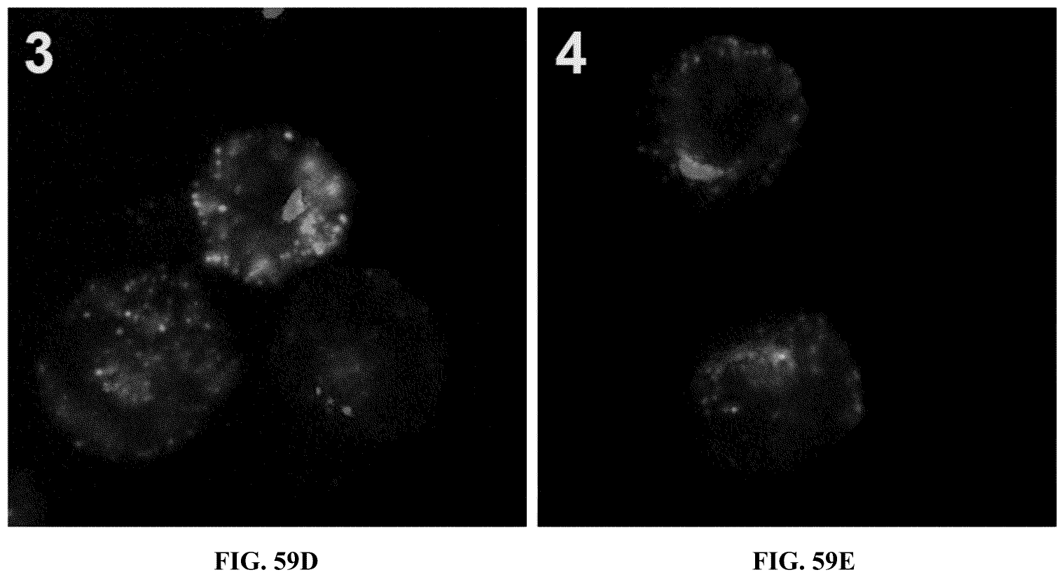

NK cells were pretreated with N-{1-[(4-chlorophenyl)methyl]piperidin-4-yl}prop-2-enamide (FIG. 56) at 5 M for 30 min. The treated NK cells were seeded onto surfaces coated with activating antibodies. Pretreatment with Compound 5814 induced lytic granule dispersion in NK cells (FIGS. 59A-59D).

5. 1-(prop-2-enamido)-N-[(thiophen-2-yl)methyl]cyclohexane-1-carboxamide (Compound 7779)

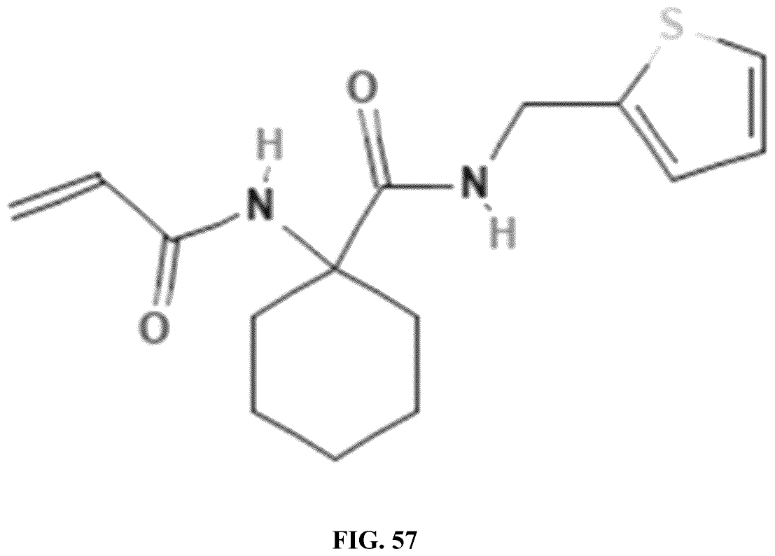

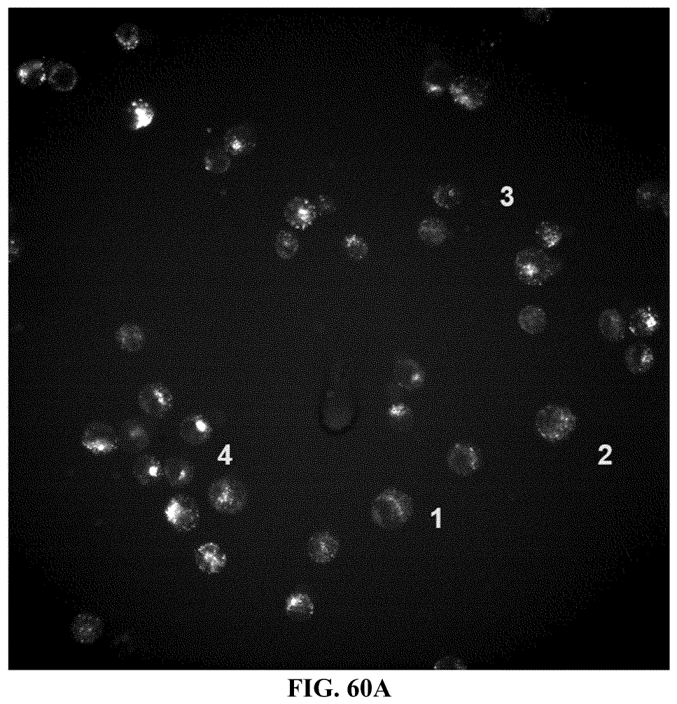

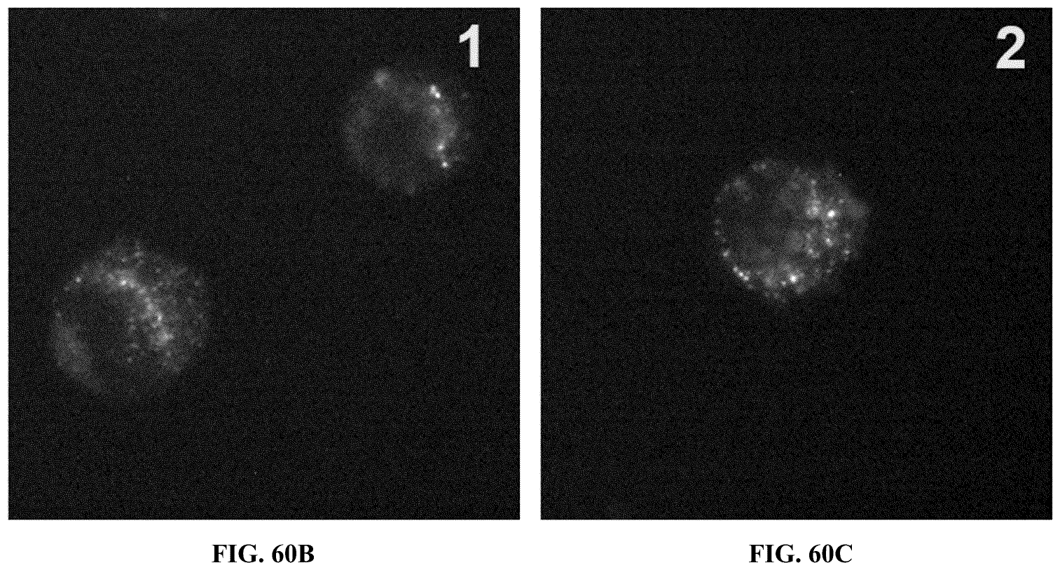

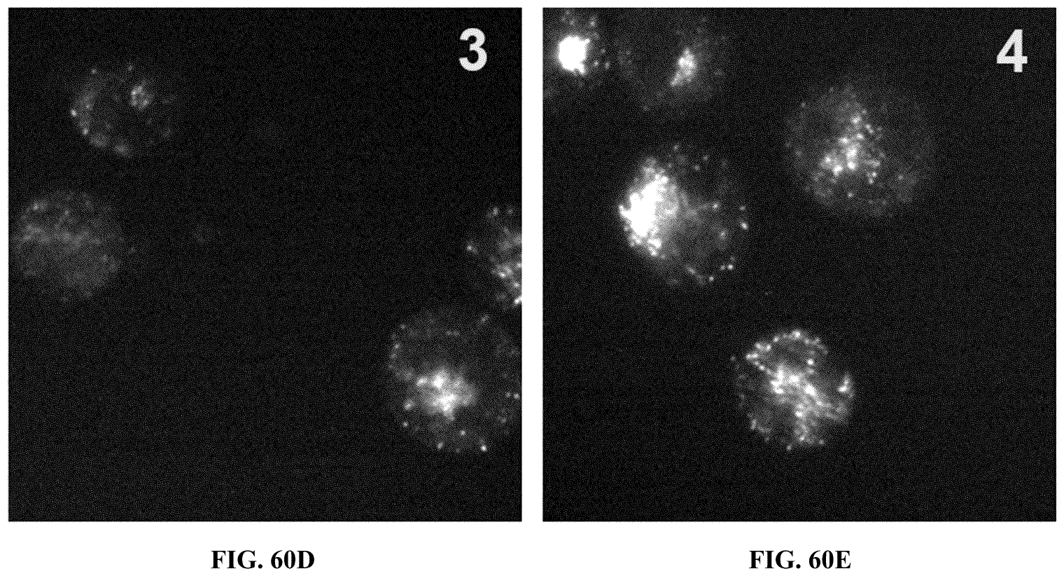

NK cells were pretreated with 1-(prop-2-enamido)-N-[(thiophen-2-yl)methyl]cyclohexane-1-carboxamide (FIG. 57) at 5 μM for 30 min. The treated NK cells were seeded onto surfaces coated with activating antibodies. Pretreatment with Compound 7779 induced lytic granule dispersion in NK cells (FIGS. 60A-60D).

6. 1-[3-(4-fluorophenyl) prop-2-enoyl]-4-hydroxypyrrolidine-2-carboxylic acid (Compound 3977)

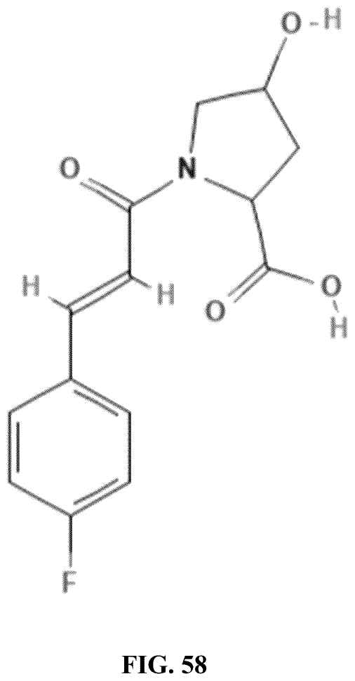

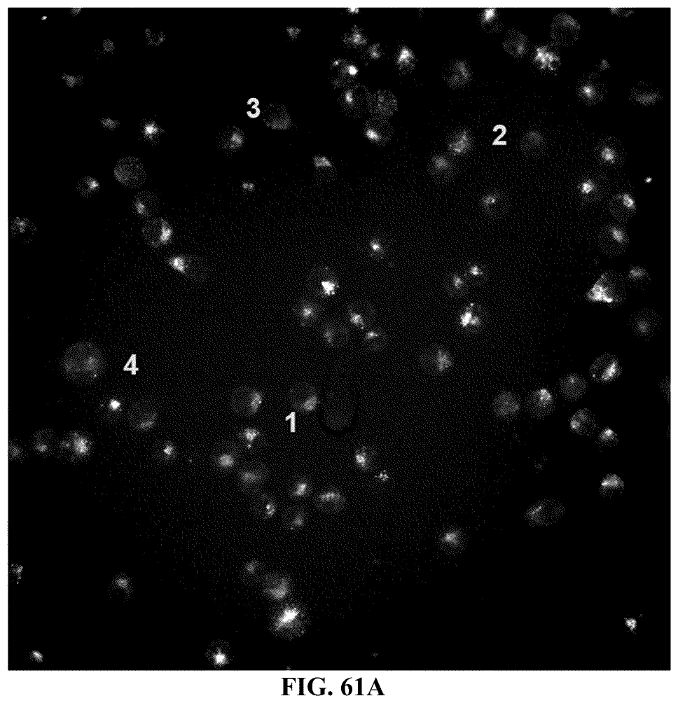

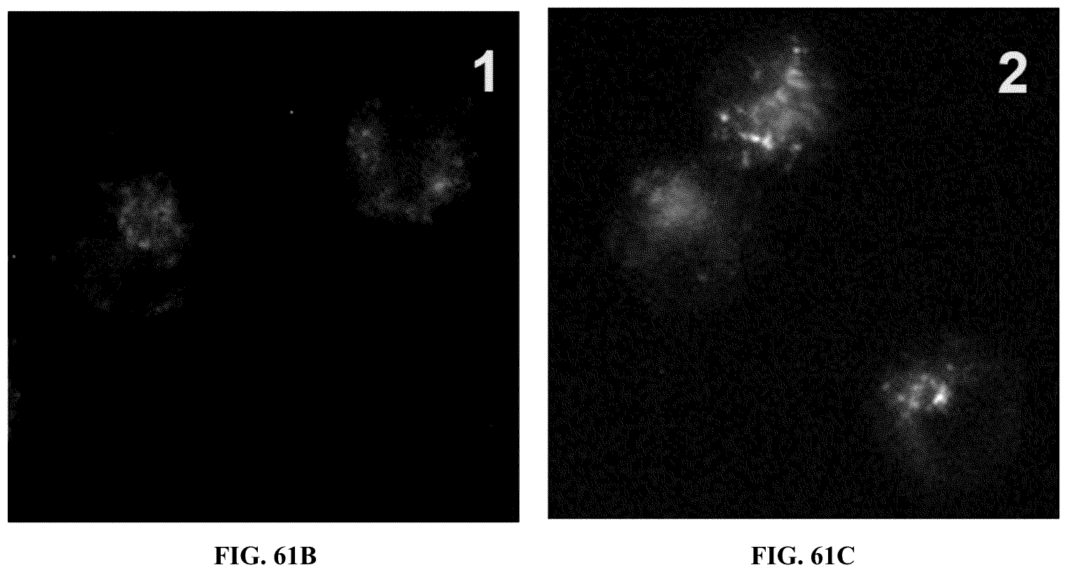

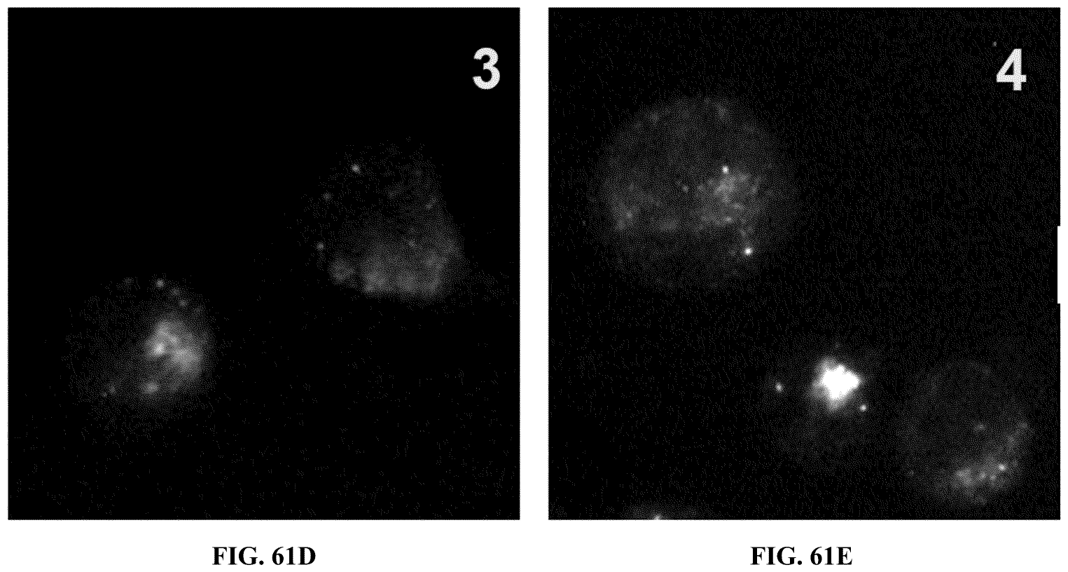

NK cells were pretreated 1-[3-(4-fluorophenyl) prop-2-enoyl]-4-hydroxypyrrolidine-2-carboxylic acid (FIG. 58) at 5 μM for 30 min. The treated NK cells were seeded onto surfaces coated with activating antibodies. Pretreatment with Compound 3977 induced lytic granule dispersion in NK cells (FIGS. 61A-61D).

### Example 4. Genes for Manipulation to Induce Lytic Granule Dispersion

### 1. Golgins

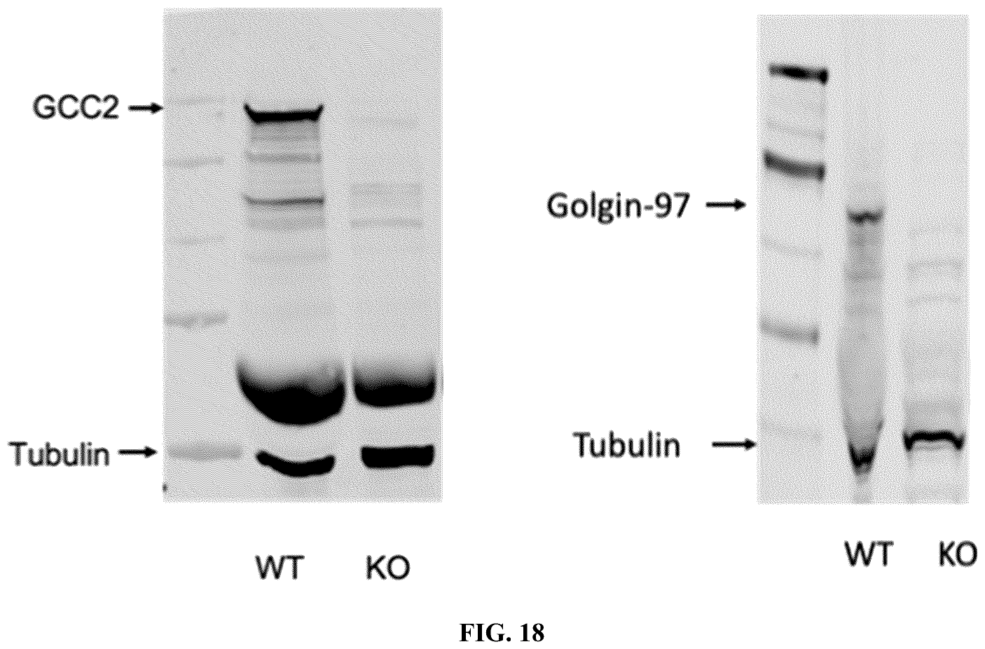

Trans golgins were knocked down using shRNA or knocked out using CRISPR Edition in NK cell lines: YTS (GCC2, G97, GCC1 and G245), NK92 (GCC2 and G97) and ex-vivo expanded Human NK cells (for GCC2 and G97) (FIG. 18).

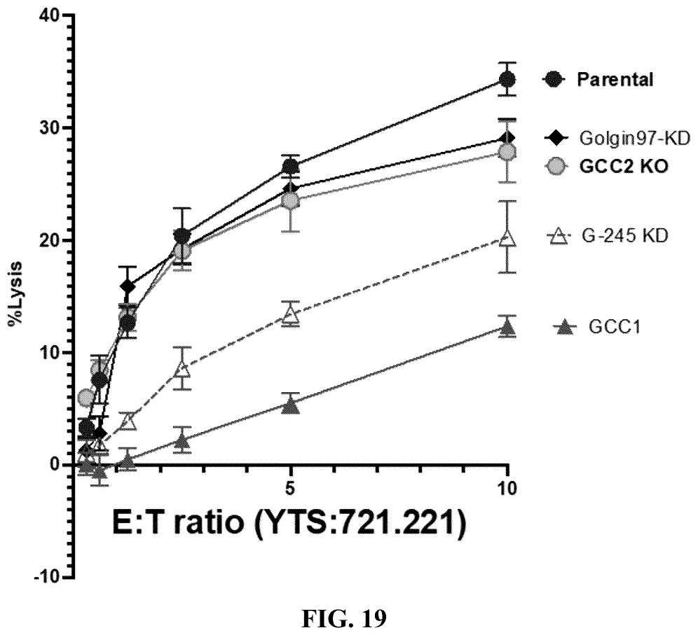

Release of Cr51 from target cells when co incubated with NK cells (cell lines or ex vivo cells) is the gold standard for assessing NK cell function. The higher counts per minute (CPM) of Cr51 indicate the more efficient killing. As shown in FIG. 19, the killing efficiency of the NK cells deficient on Golgin-97 and GCC2 within normal range, suggesting the NK cells are still efficient in killing target cells. The killing efficiency of the NK cells deficient on GCC1 and G-245 are decreased, but still present (FIG. 19).

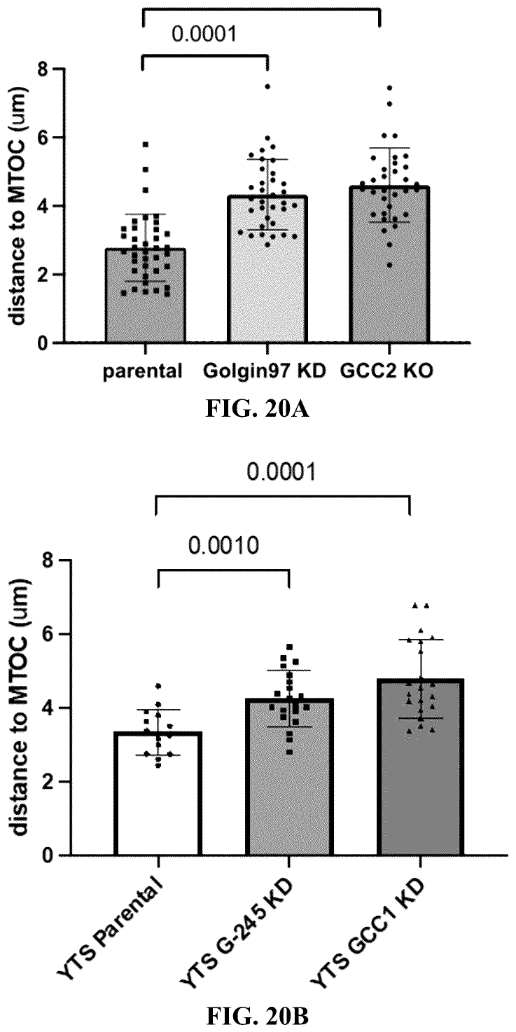

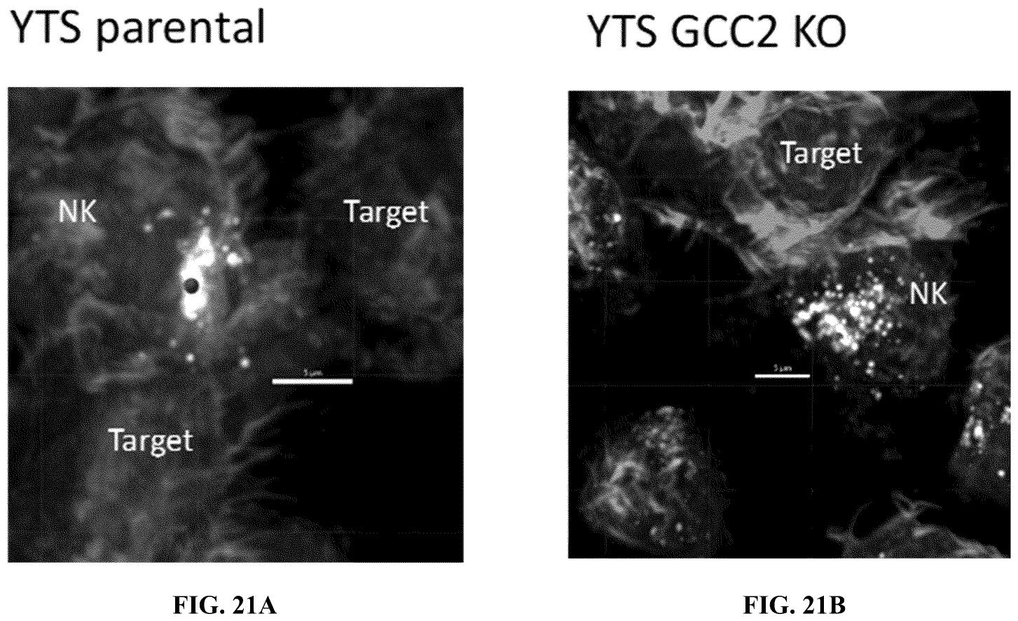

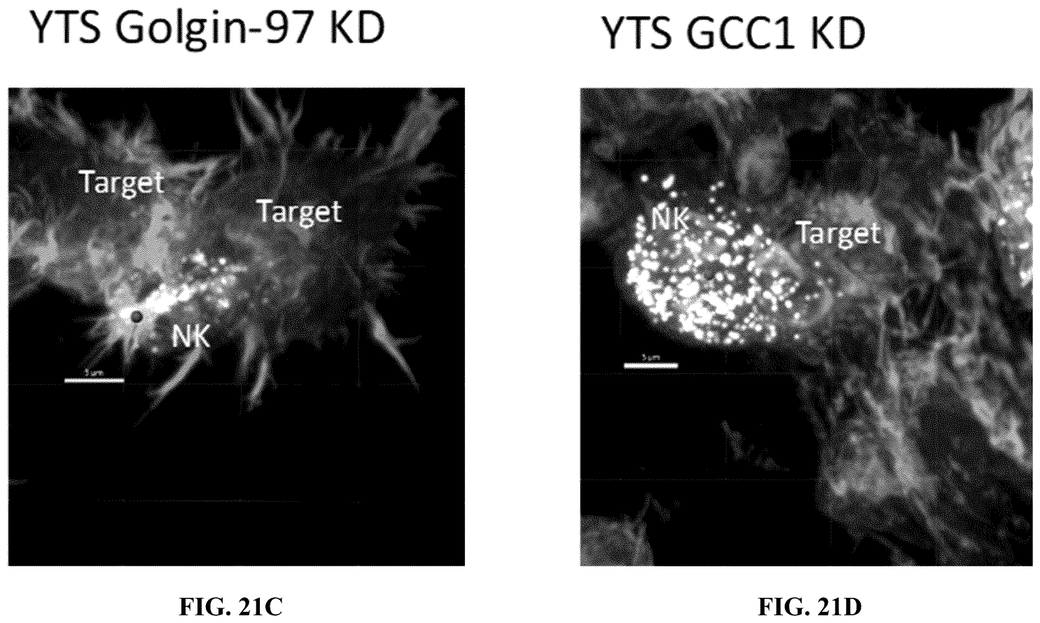

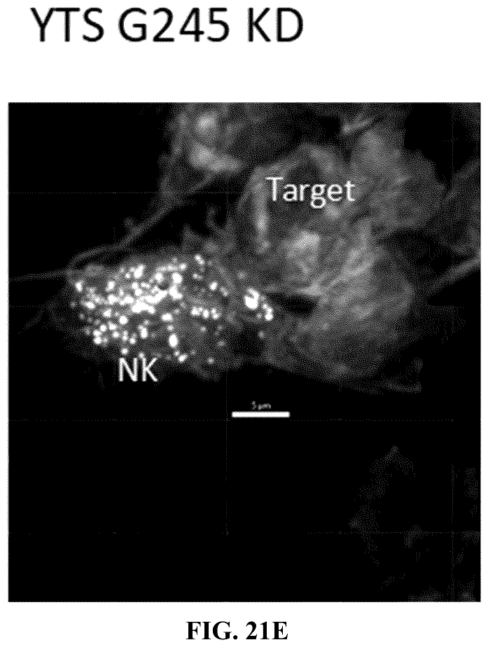

NK cytolytic granules are known to contain perforin, which can be used to visually track the position of the granules in a given moment and can be used to assess granule dispersion. YTS cells were incubated with targets 721.221 for 60 min and stained for perforin (granules marker) and tubulin, to track the MTOC. The average distance between the granules and the MTOC was measured on the software Imaris. YTS cells deficient for the golgins show increase in the distance of the granules to the MTOC during the conjugation with Target cell, suggesting an increase granule dispersion (FIGS. 20A, 20, and 21A-21E).

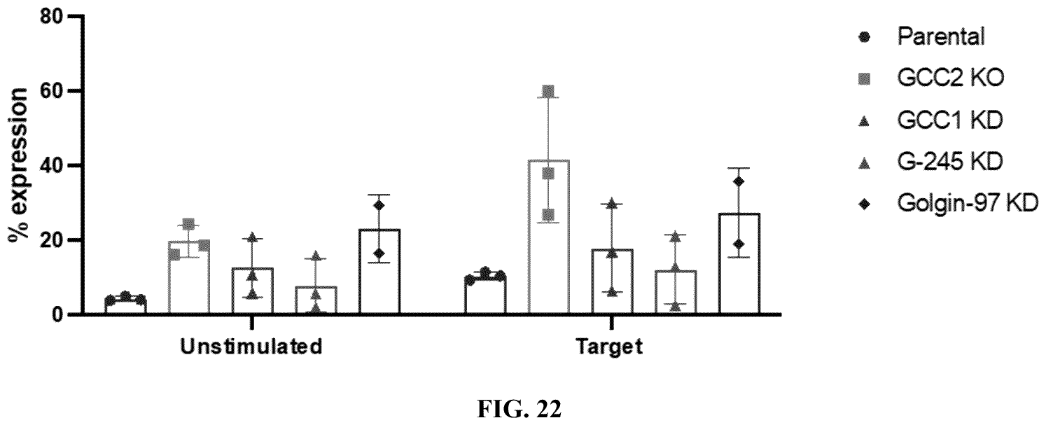

YTS cells were stimulated for 60 min with Target cells 721.221, stained for CD107a and analyzed by FACS. GCC2 KO cells and Golgin-97 KD cells show a significant increase on degranulation, with GCC1 KD and G-245 KD showing a residual increase on degranulation (FIG. 22).

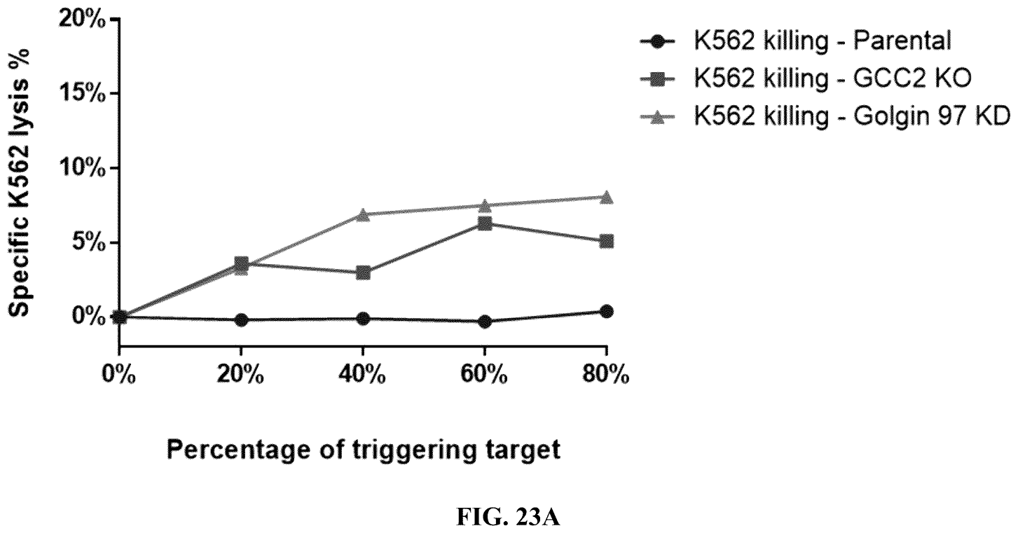

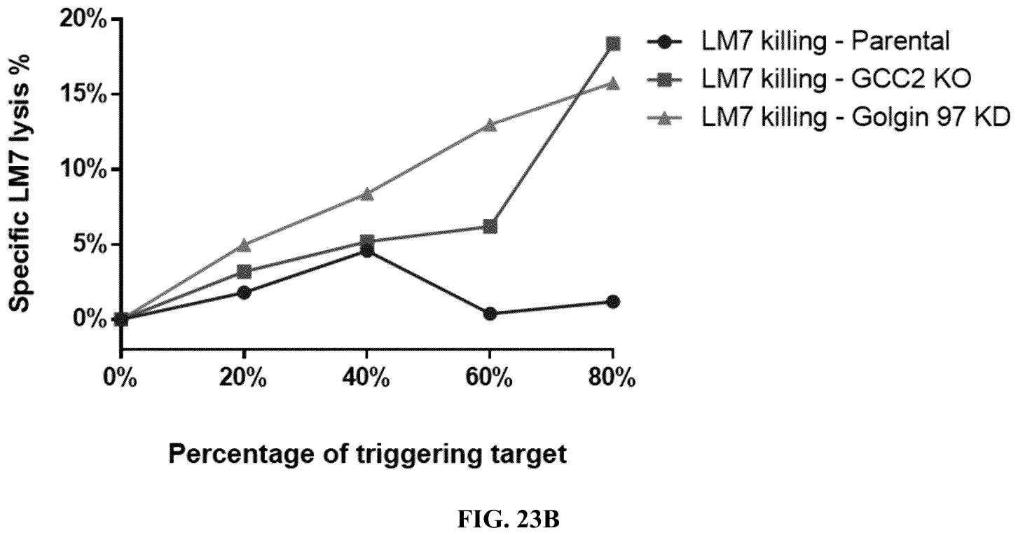

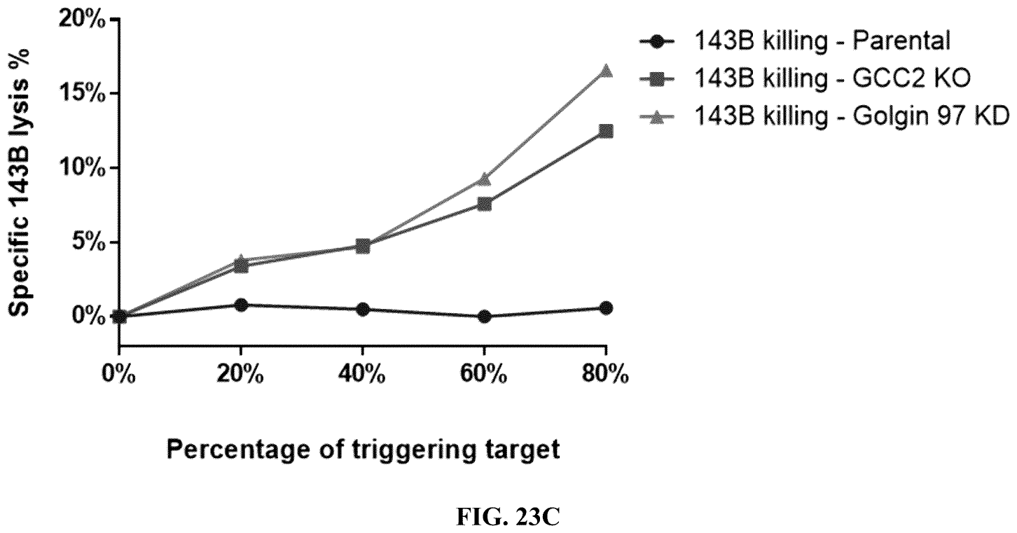

A TheCOS model was created with either GCC2 KO and Golgin 97 KD YTS cells to study the killing efficiency of bystander K562 cells (FIG. 23A), bystander LM7 cells (FIG. 23B), and bystander 143B cells (FIG. 23C).

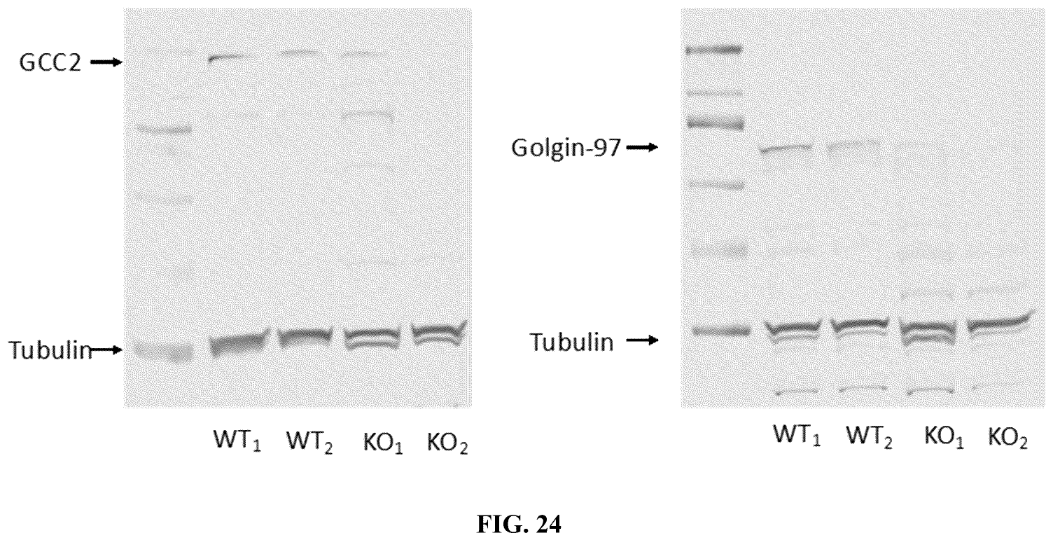

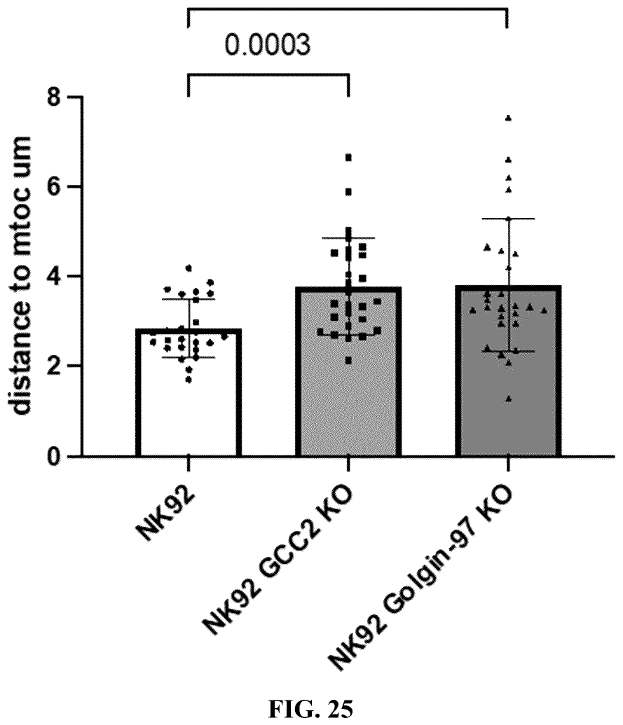

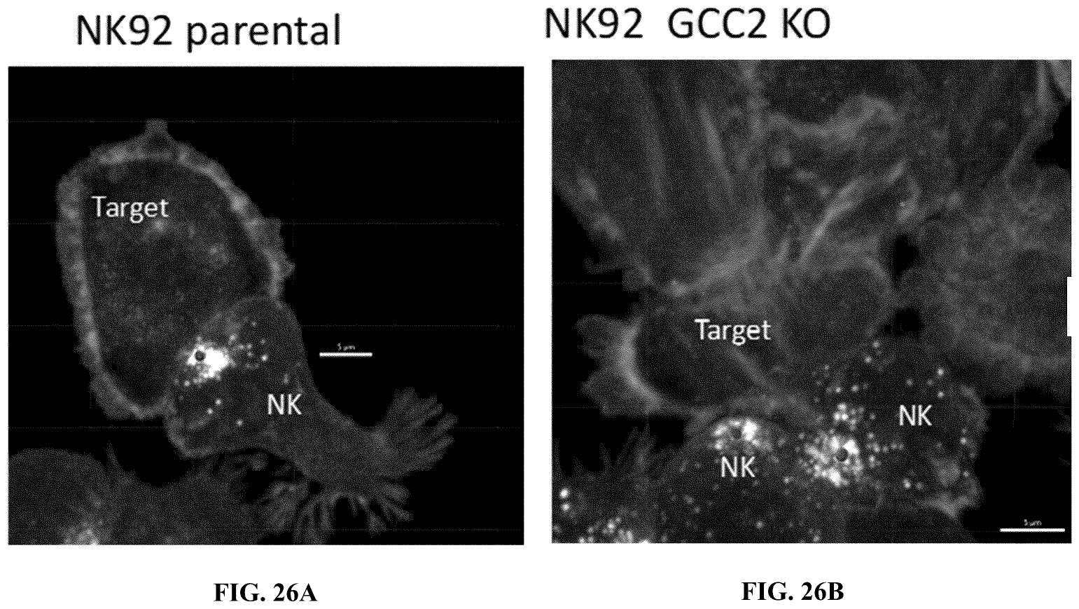

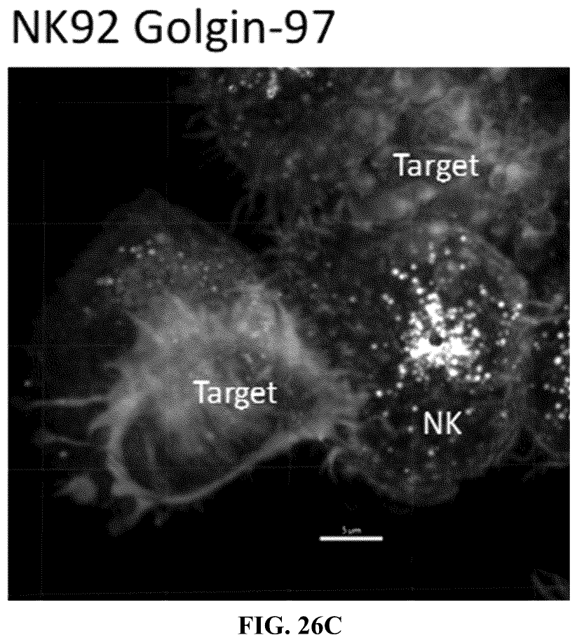

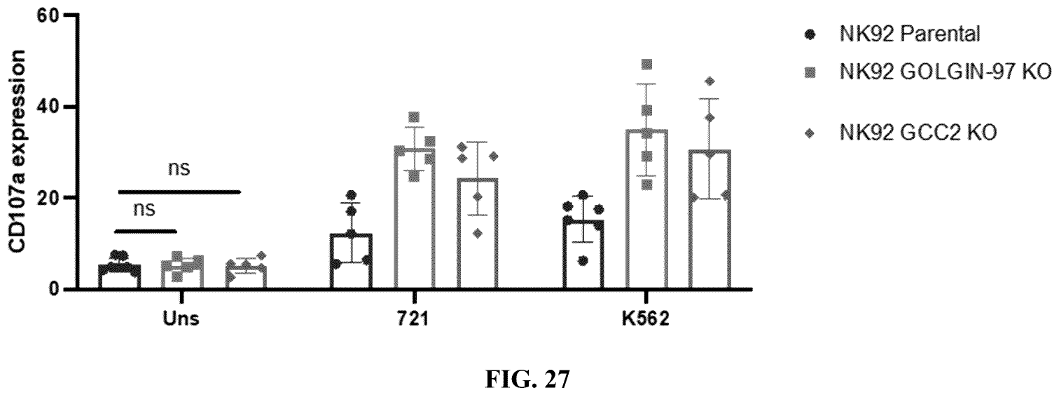

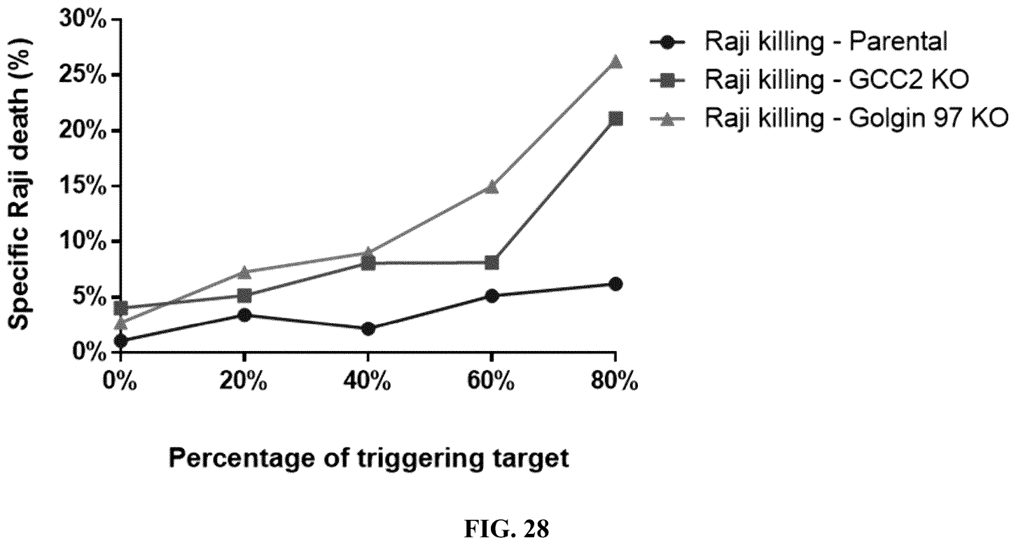

The above experiments were replicated in a second NK cell line, NK92. GCC2 KO and Golgin 97 KO NK92 cells were created (FIG. 24). These cells show an increase in the distance of the granules to the MTOC during the conjugation with a target cell, suggesting an increase granule dispersion (FIGS. 25 and 26A-26C). NK92 cells with GCC2 or Golgin 97 knocked out were stimulated for 60 min with target cells 721.221 or K562 and then stained for CD107a and analyzed by FACS. GCC2 KO cells and Golgin-97 KO cells show an increase on degranulation (FIG. 27). A TheCOS model was created either GCC2 KO and Golgin 97 KO NK92 cells to study the killing efficiency of bystander Raji cells (FIG. 28).

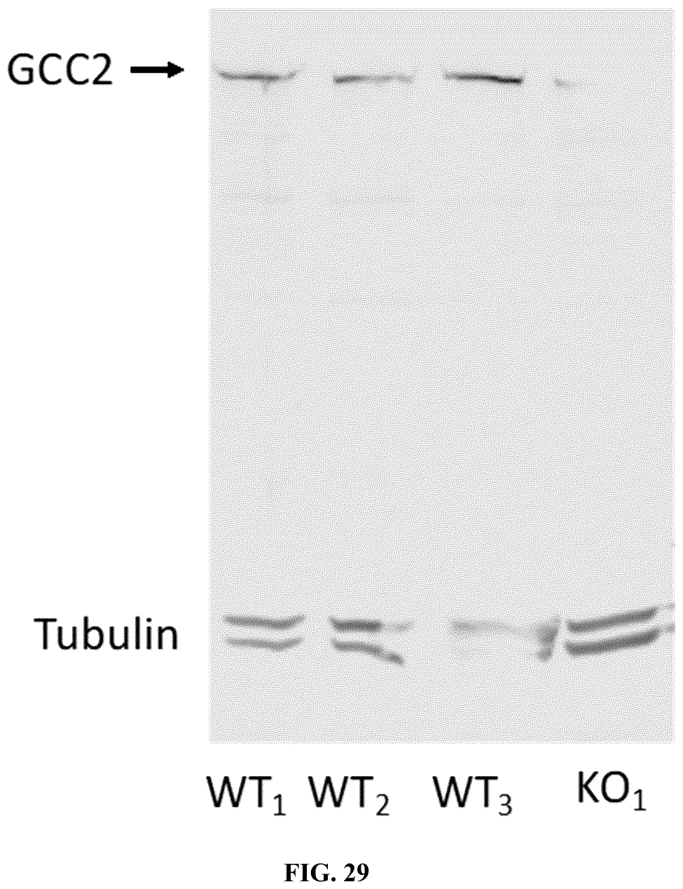

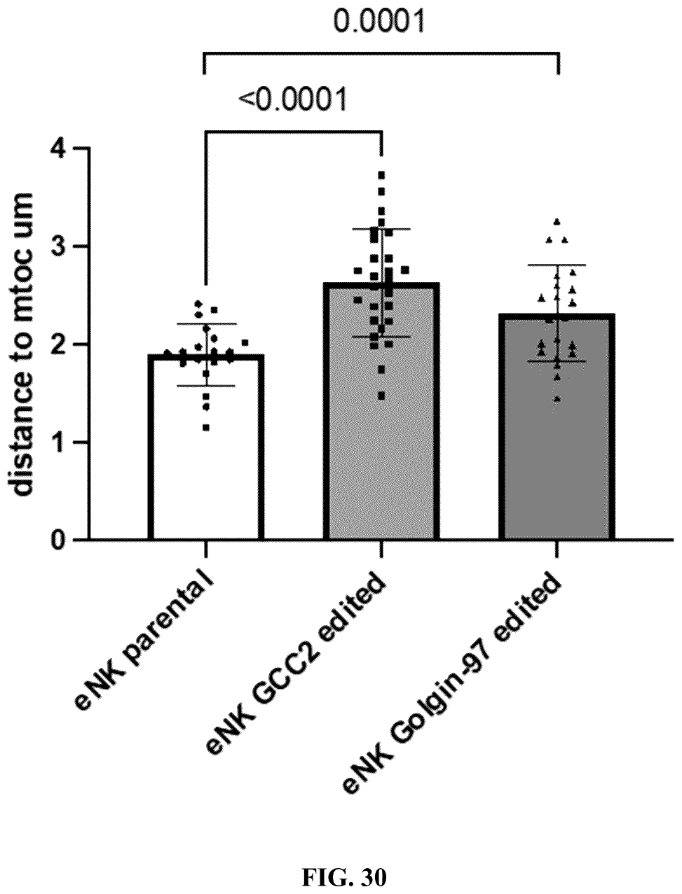

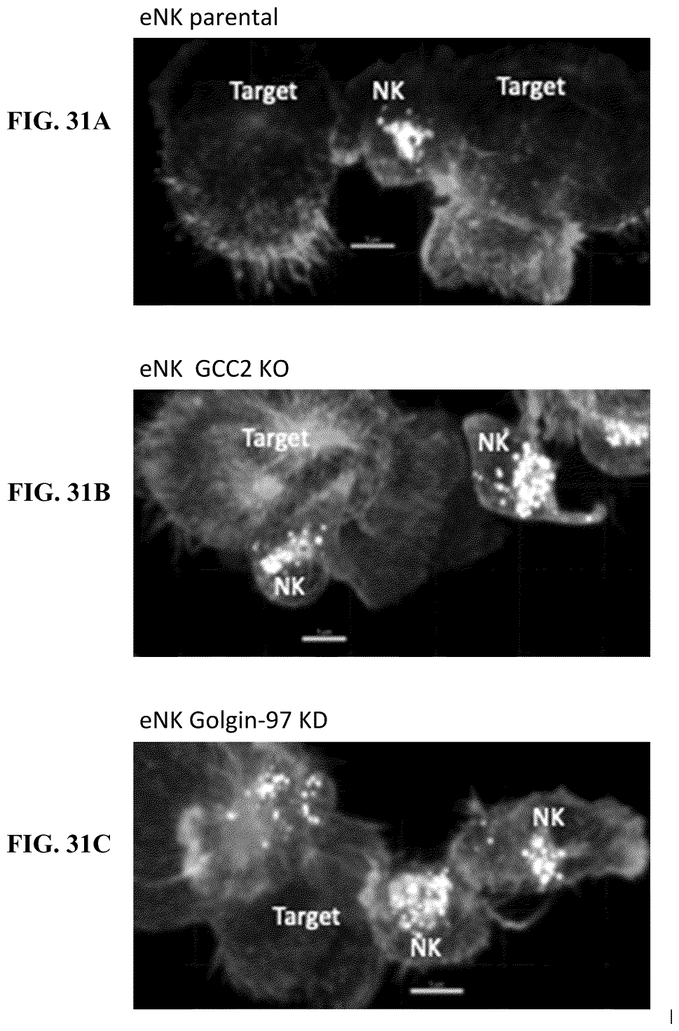

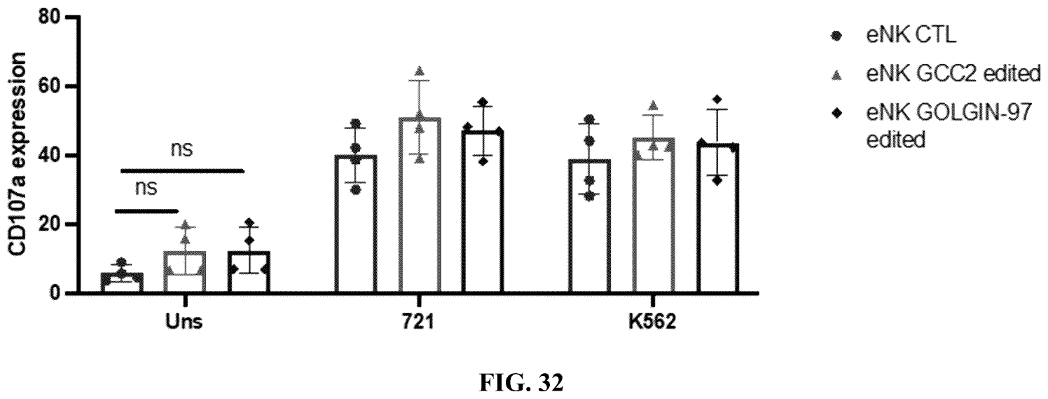

For ex vivo NK cell studies, NK cells from a healthy donor were isolated using Rossette-sep method and cultured on specific NK media (NK-MACS from Miltenyi). The cells were then CRISPR edited for GCC2 (FIG. 29) and Golgin-97 (data not shown). eNK cells were incubated with targets K562 for 60 min and stained for perforin (granules marker) and tubulin, to track the MTOC. The average distance between the granules and the MTOC was measured using the software Imaris. Edited cells deficient for GCC2 and Golgin-97 show an increase in the distance of the granules to the MTOC during the conjugation with Target cell, suggesting an increase granule dispersion (FIGS. 30, 31A-31C, and 32).

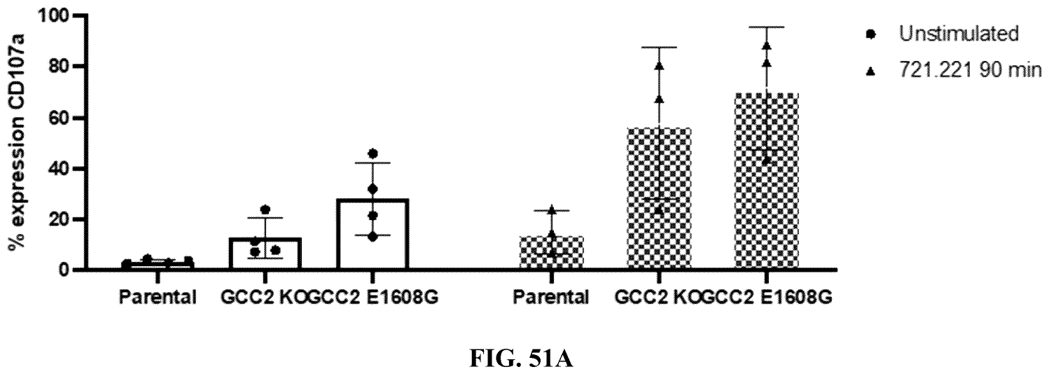

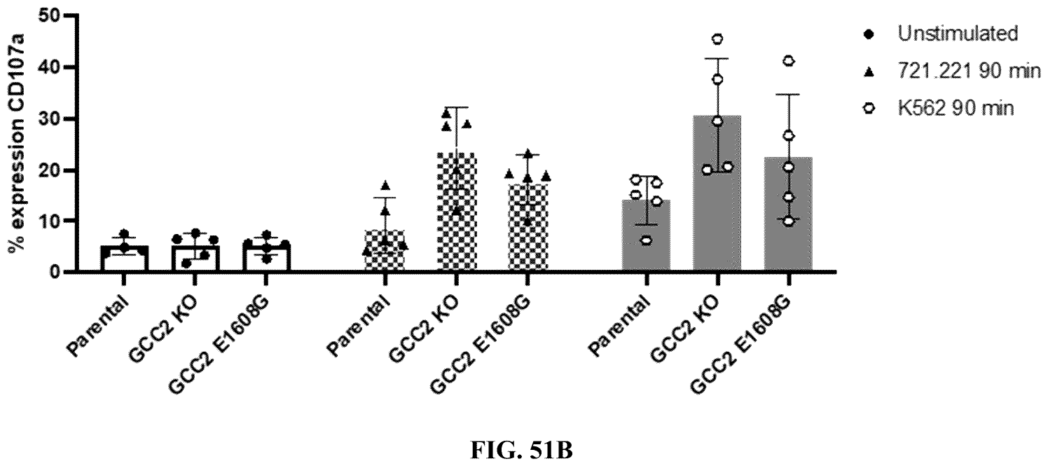

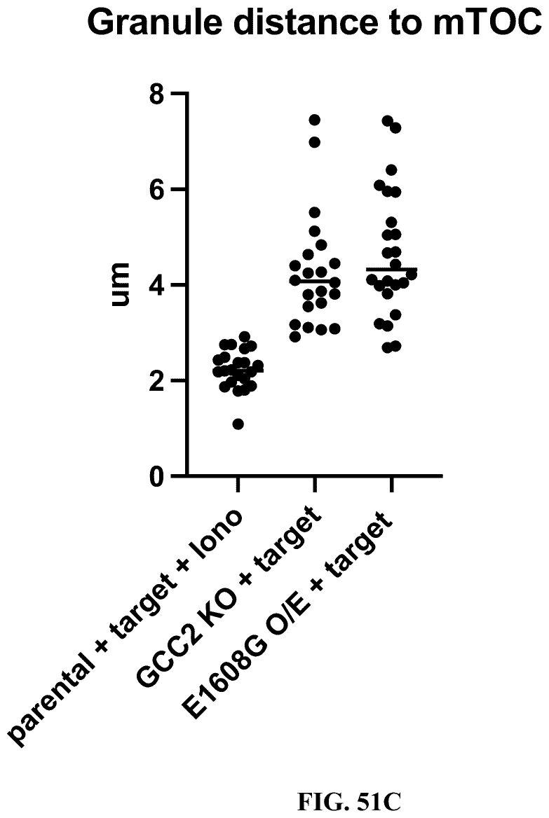

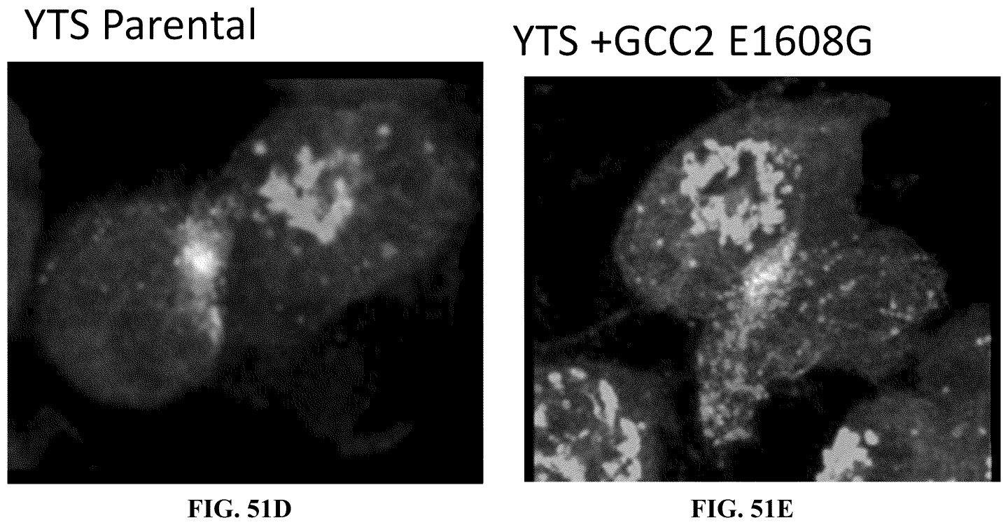

Inhibiting the function of golgins by knocking in the expression of the vesicle modifying protein is also sufficient to cause degranulation. For example, knocking in the expression of GCC2 in YTS cells and NK92 cells using site directed mutagenesis of the gene at residue 1608 (Glutamic Acid to Glycine) results in a similar increase on the degranulation in both cells (FIGS. 51A and 51B). The mutated GCC2 gene was subcloned on plasmid pCDH-CMV-MCS-EF and transfected into the two NK cell lines before being conjugated with K562 target cells and/or 721.221 target cells for 90 minutes before measuring the concentration of CD109a. GCC2 knocked in YTS cells have increased granule distance to MTOC after conjugation with a target cell at a level similar to YTS cells with GCC2 expression knocked out (FIG. 51C),

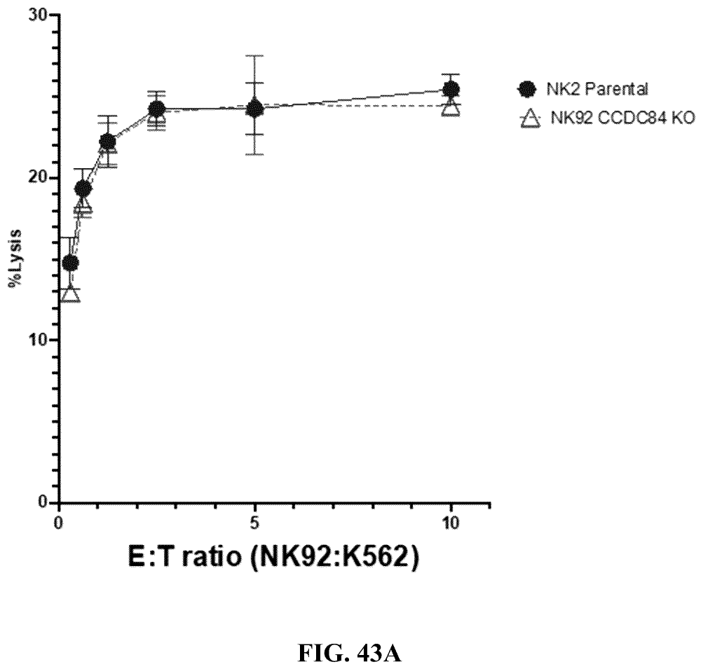

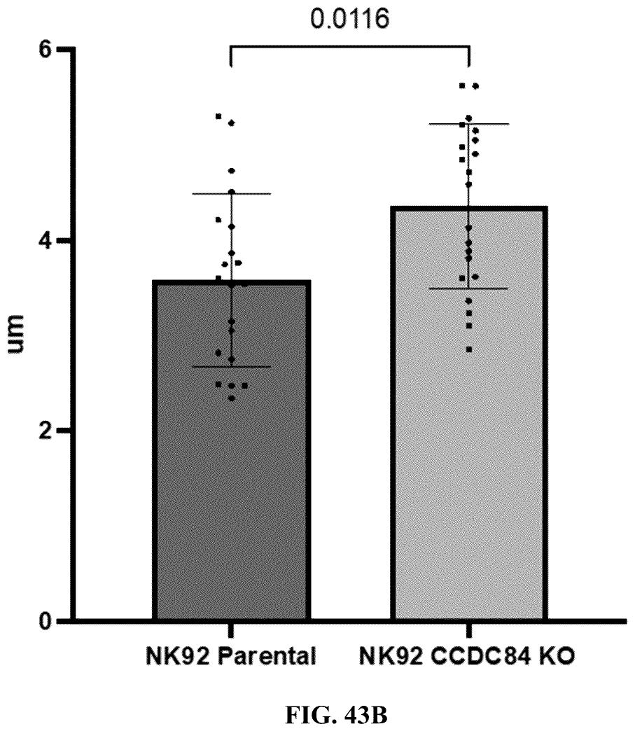

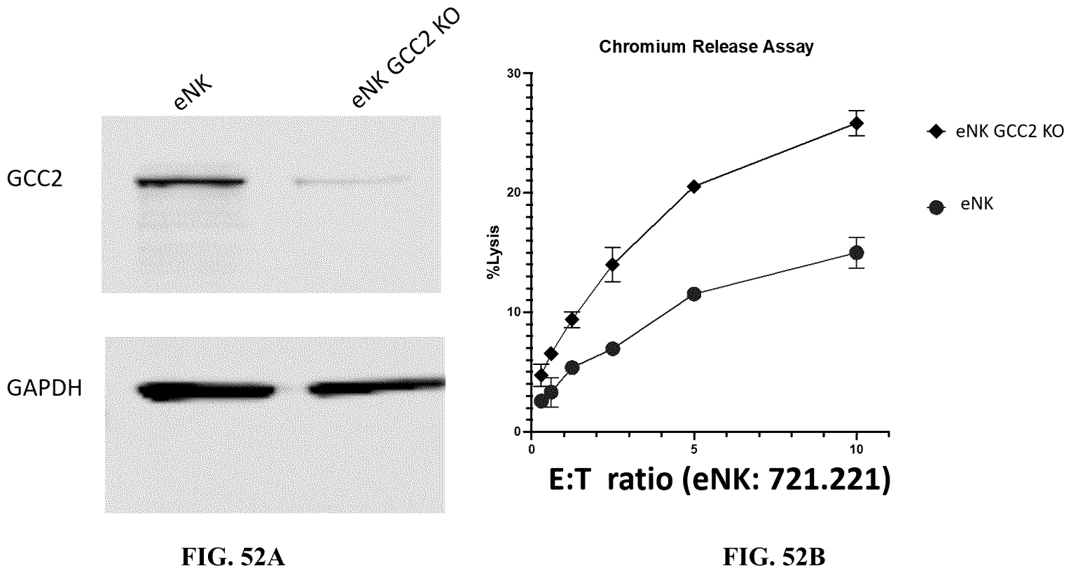

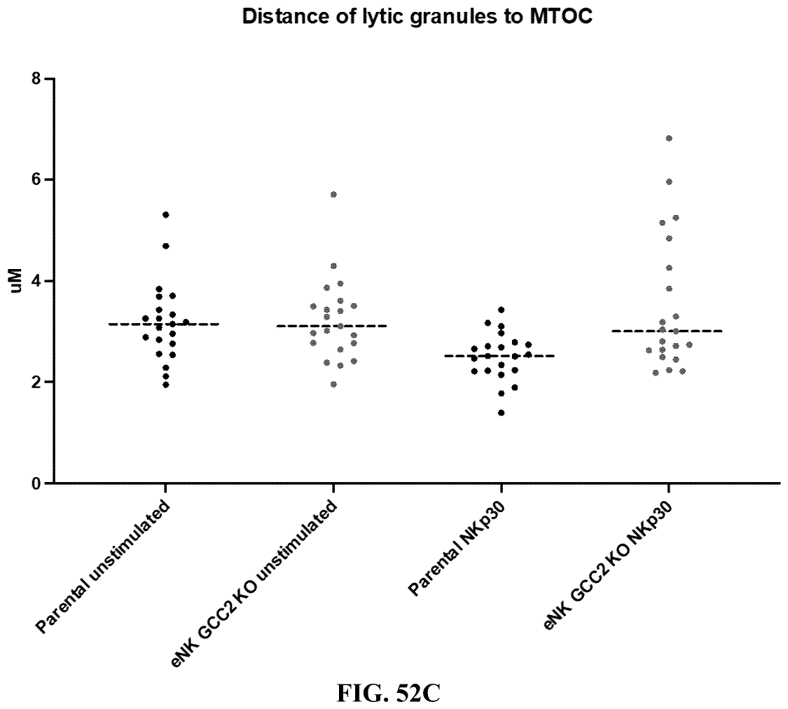

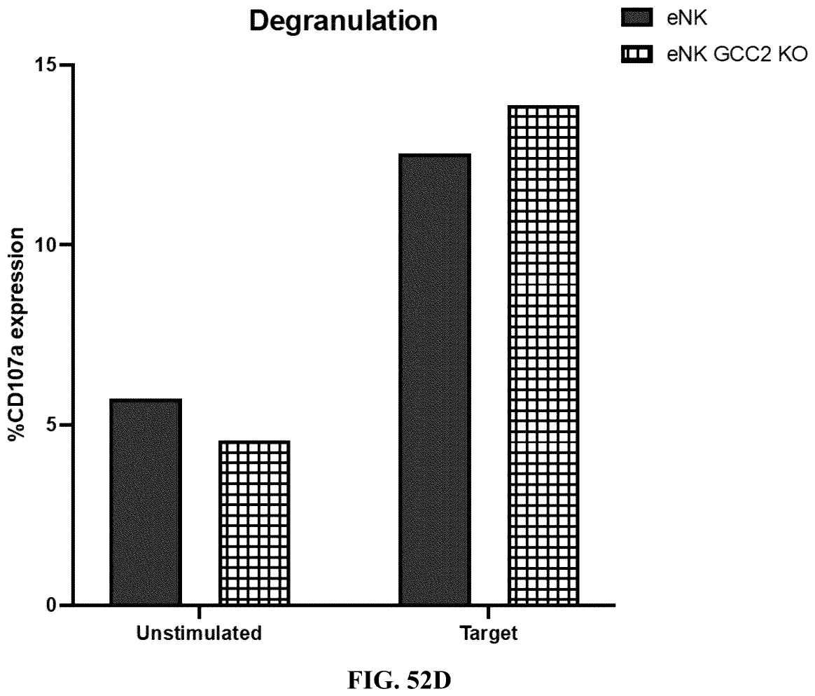

In another example, GCC2 was knocked out using CRISPR/Cas9 edition using ex vivo expanded NK cells isolated from a healthy donor. Western blot demonstrates diminished GCC2 protein in the edited eNK cells relative to a GADH loading control (FIG. 52A). Release of Cr51 from target cells when co-incubated with NK cells (cell lines or ex vivo cells) is the gold standard for assessing NK cell function. The higher CPM of ⁵¹Cr indicate the more efficient killing, for this, NK92 cells were incubated with targets K562 (previously marked with ⁵¹Cr) in different ratios (as showed on the FIG. 43A), during 4 hours. The killing efficiency of eNK GCC2 KO cell towards 721.221 target cells is increased compared to unedited eNK cells indicating that eNK GCC2 KO cells are very efficient killers of target cells (FIG. 52B). eNK cells deficient for GCC2 show an increasing trend in the distance of the lytic granules to the MTOC on an activated glass surface (anti-NKp30 coating) (FIG. 52C) indicating increased granule dispersion for eNK GCC2 KO cells. eNK cells were stimulated for 60 min with target cells 721.221, stained for CD107a and analyzed by FACS. The analysis of the eNK GCC2 KO cells shows an increasing trend in the level of degranulation when compared to unedited eNK cells (FIG. 52D).

### 2. BIG1

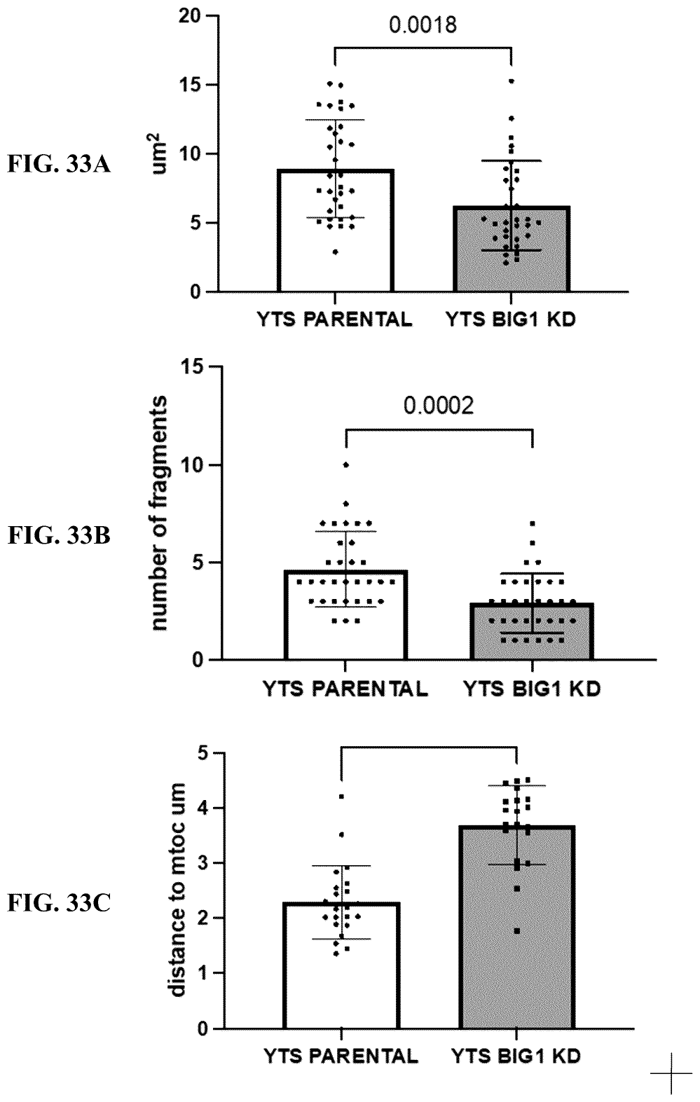

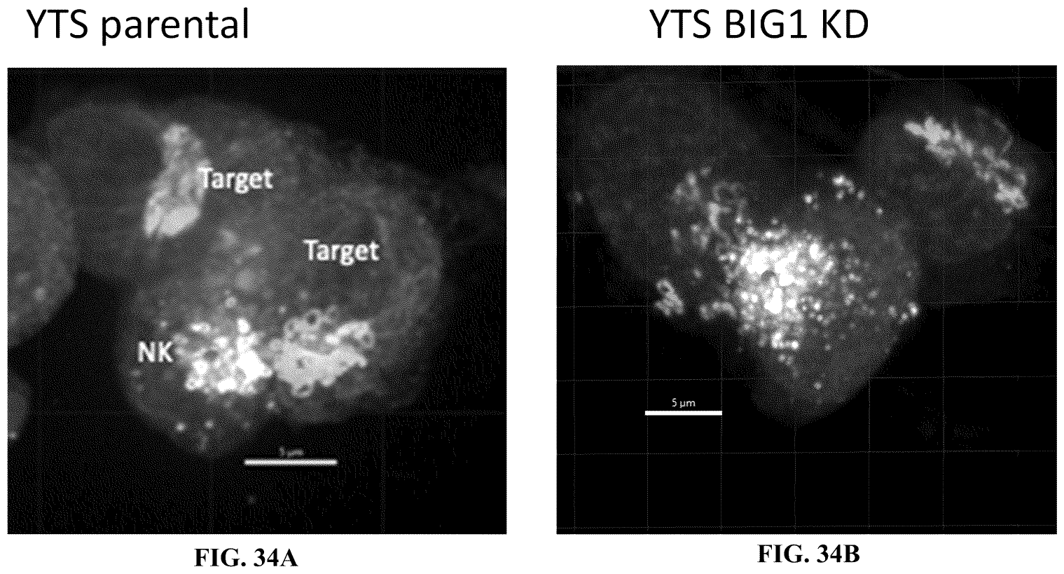

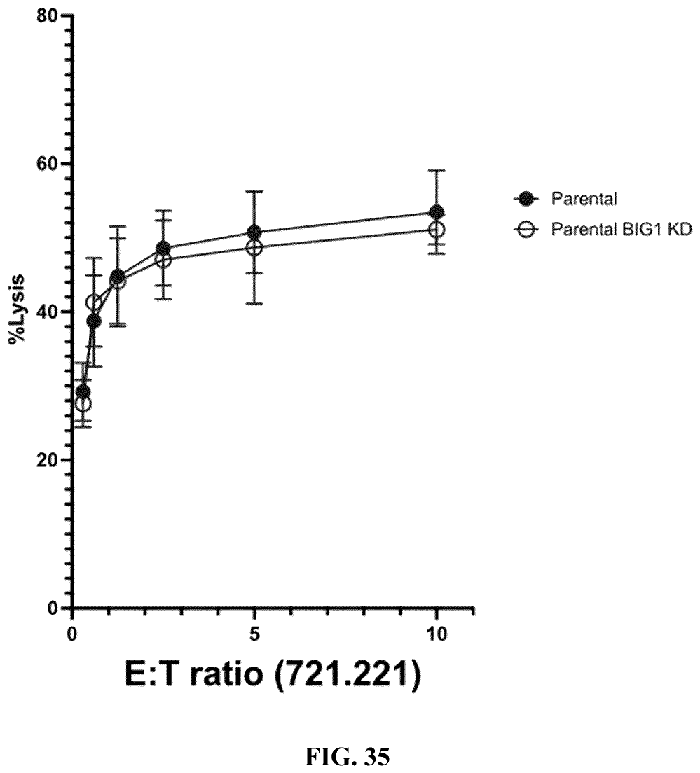

BIG1 is an ADP-ribosylation factor that has been previously reported to induce Golgi fragmentation. YTS cells were transduced with lentivirus expressing BIG1 specific shRNA and selected using Puromycin. YTS BIG1 KD cells were incubated with 721.221 target cells for 60 min and stained for perforin (granules marker) and tubulin, to track the MTOC. The average distance between the granules and the MTOC was measured on the software Imaris. The cells were also stained for Giantin, a specific Golgi protein, and the number of Golgi fragments were measured using the software Imaris. The level of fragmentation on BIG1 KD was higher than expected and the Golgi fragments were smaller than the cutoff value used (FIGS. 33A and 33B). YTS BIG1 KD cells show increase in the distance of the granules to the mTOC during the conjugation with target cell, suggesting an increase granule dispersion (FIGS. 33C, 34A, and 34B). The killing efficiency of the YTS BIG1 KD are within normal range, suggesting the YTS cells are still efficient in killing target cells (FIG. 35).

3. RABGGTA (Rab Geranyl-Geranyltransferase Subunit Alpha) RABGGTA is an enzyme that catalyzes the transfer of a geranylgeranyl moiety from geranylgeranyl diphosphate to both cysteines present on the C-terminal region of Rab proteins (sequence-XXCC, -XCXC and -CCXX). Geranylgeranyl modification of Rab proteins is important for the membrane targeting of these proteins. In its absence the Rab proteins are predicted to be in soluble form in the cytoplasm. Rab proteins are key players in regulating trafficking of vesicles (including, but not limited to lytic granules) to different compartments of the cell, including movement to the plasma membrane.

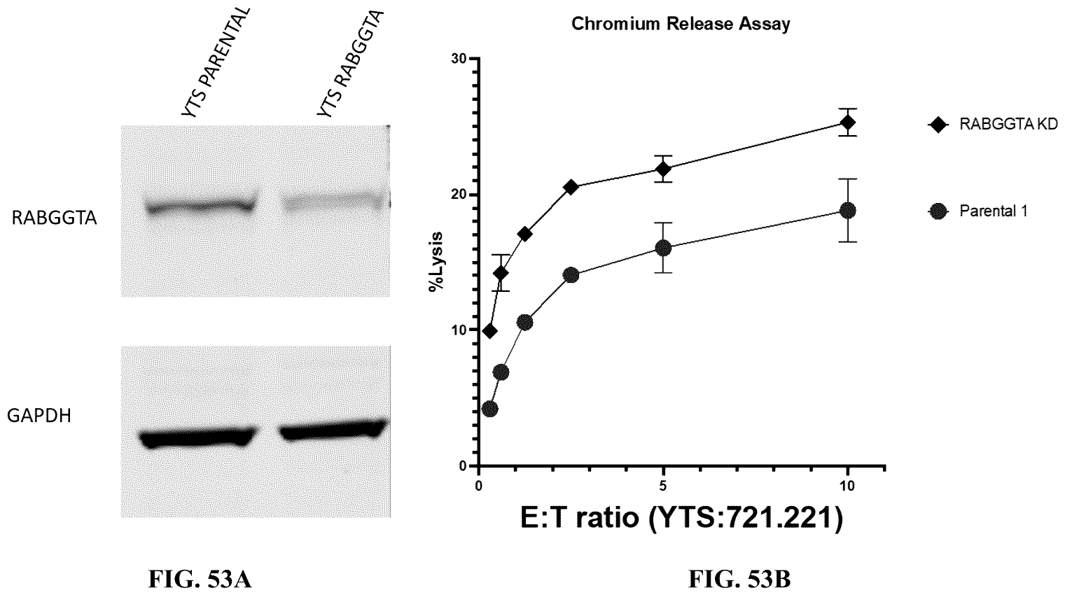

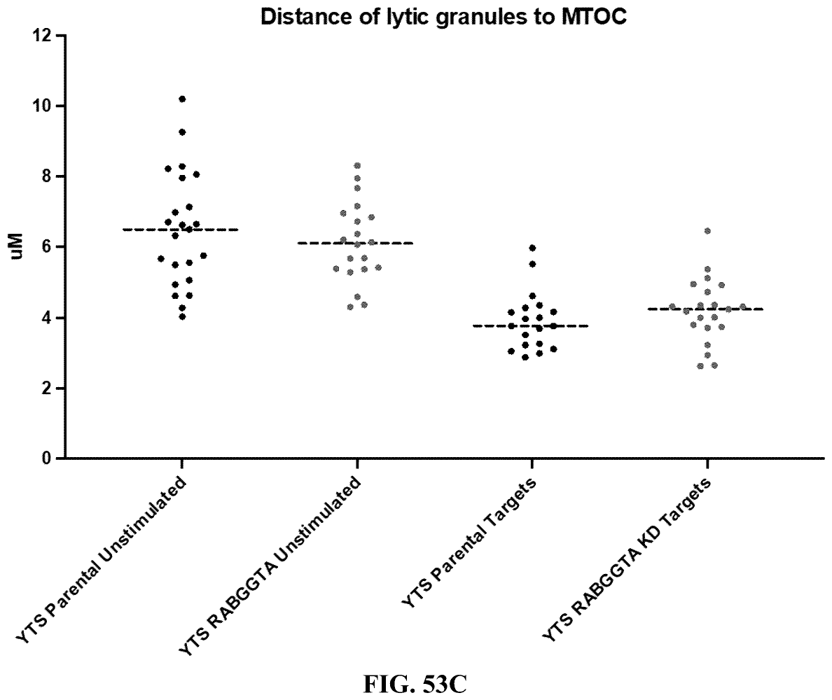

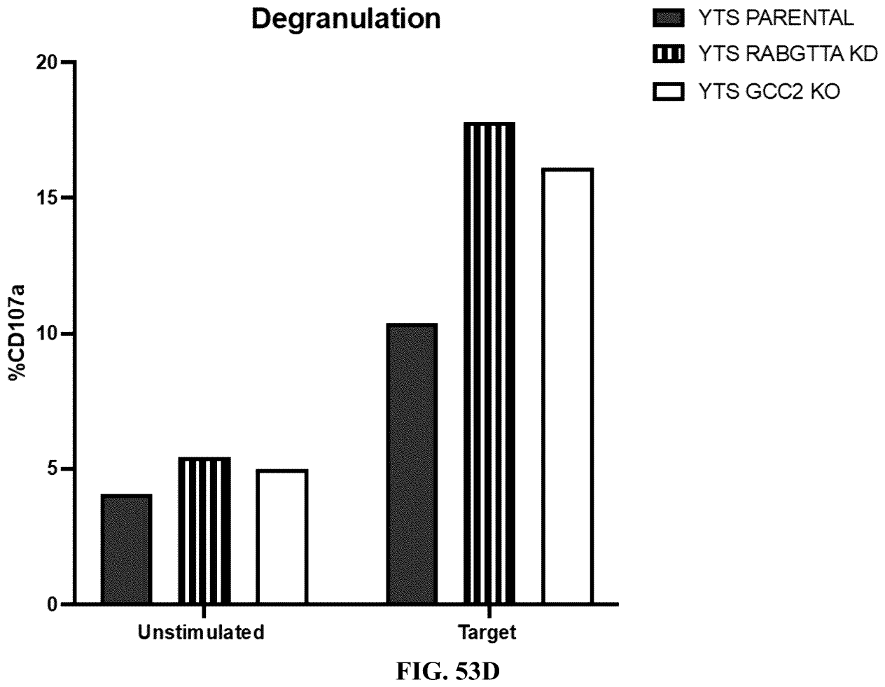

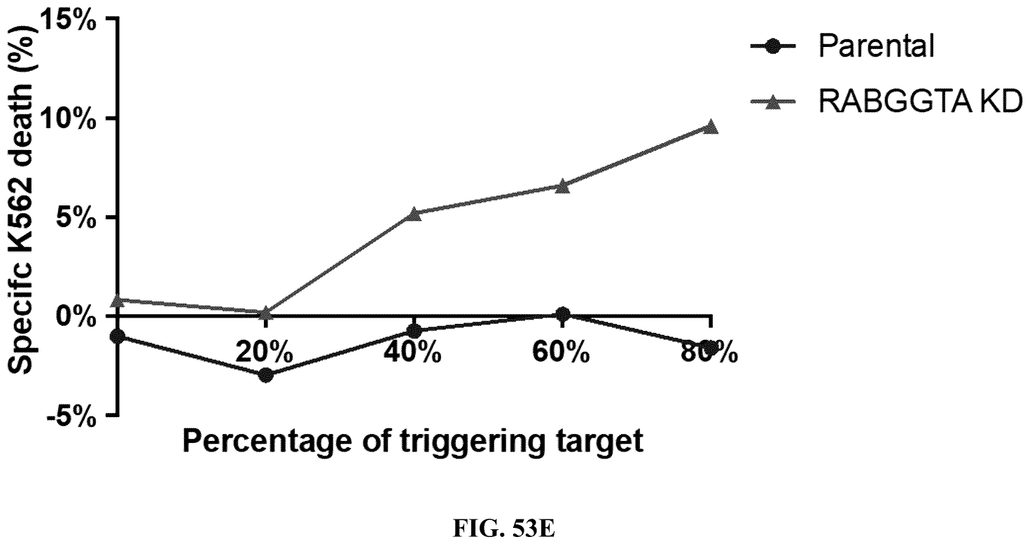

RABGGTA was edited using CRISPR/Cas9 and a bulk population of cells analyzed. RABGGTA protein level is decreased compared to parental cells (approx. 60% knock down efficiency) in bulk cell population (RABGGTA KD NK cells) relative to GAPDH loading control as demonstrated by Western Blot (FIG. 53A). The killing efficiency of RABGGTA KD NK cells towards 721.221 target cells is increased compared to YTS parental NK cells which indicates that RABGGTA KD cells are very efficient killers of target cells (FIG. 53B). YTS cells depleted for RABGGTA show an increasing trend in the distance of the granules to the MTOC during conjugation with target cells, suggesting an increase in granule dispersion (FIG. 53C). YTS cells were stimulated for 60 min with target cells 721.221, stained for CD107a and analyzed by FACS. RABGGTA KD cells show an increased level of degranulation when compared to YTS parental cells. YTS GCC2 KO cells function as positive control for increased degranulation (FIG. 53D).

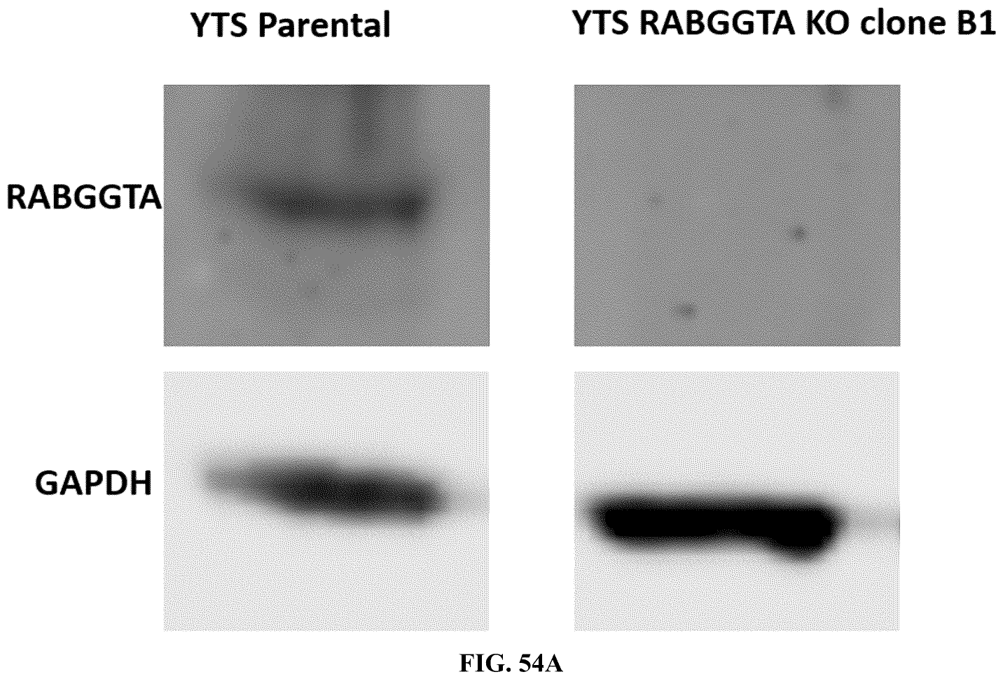

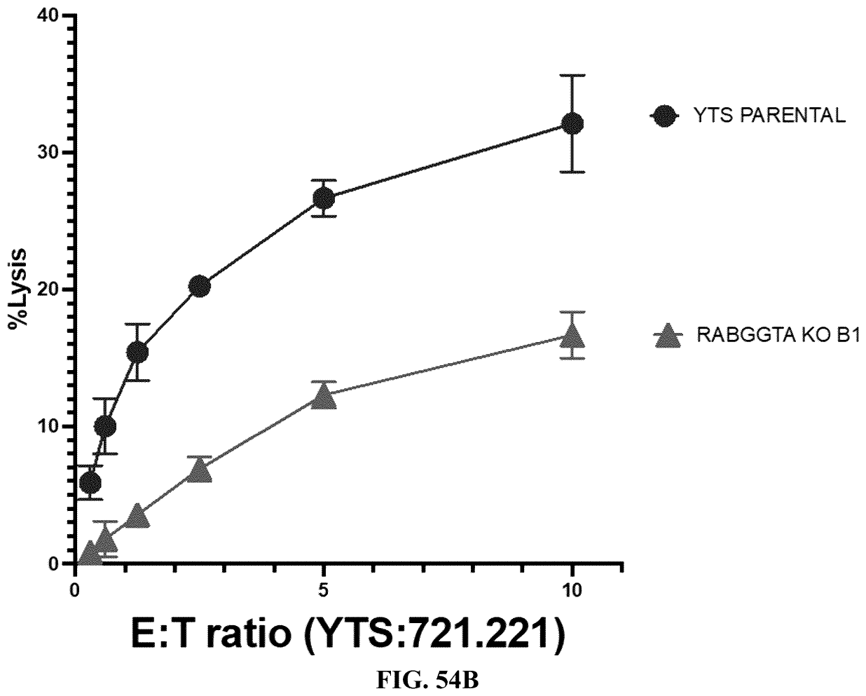

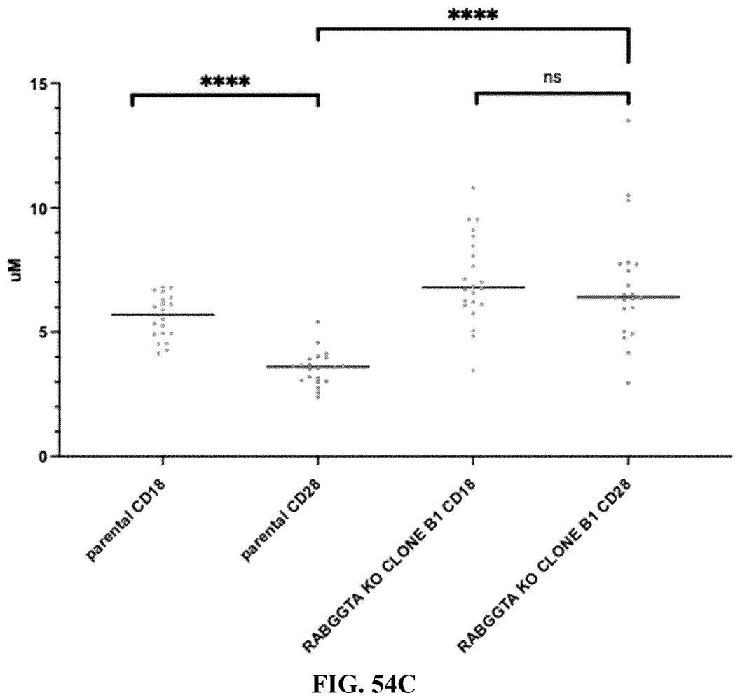

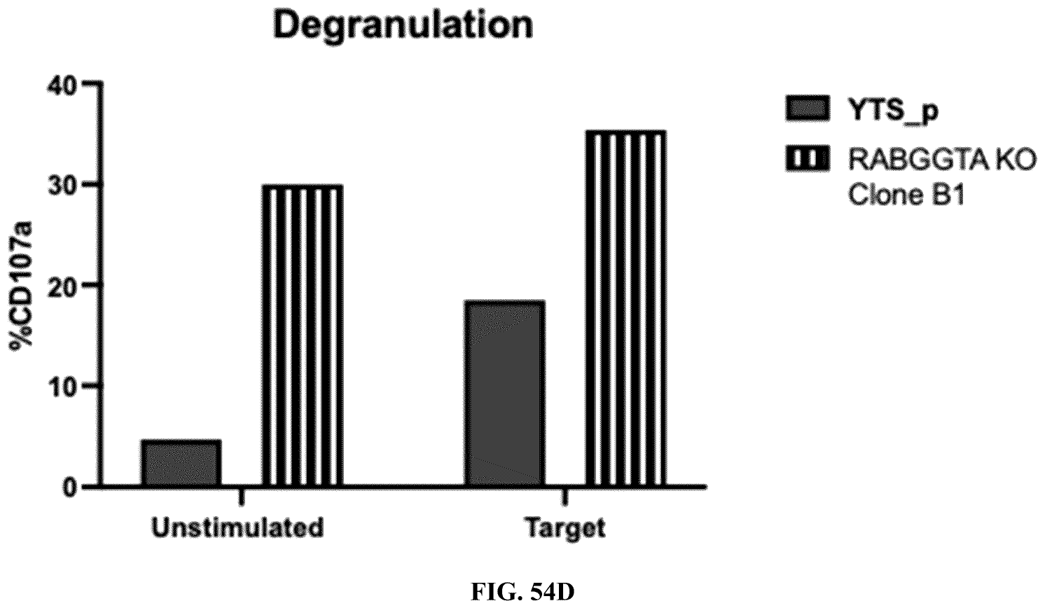

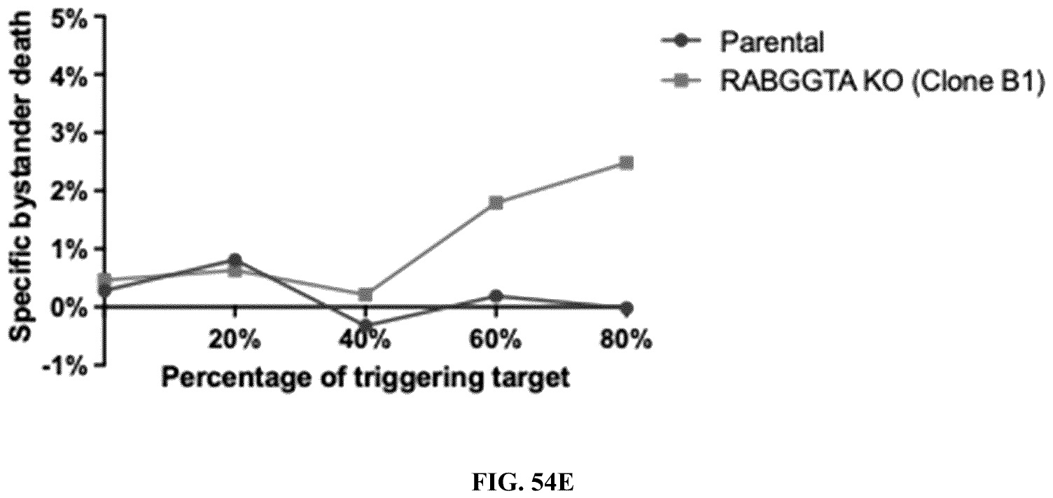

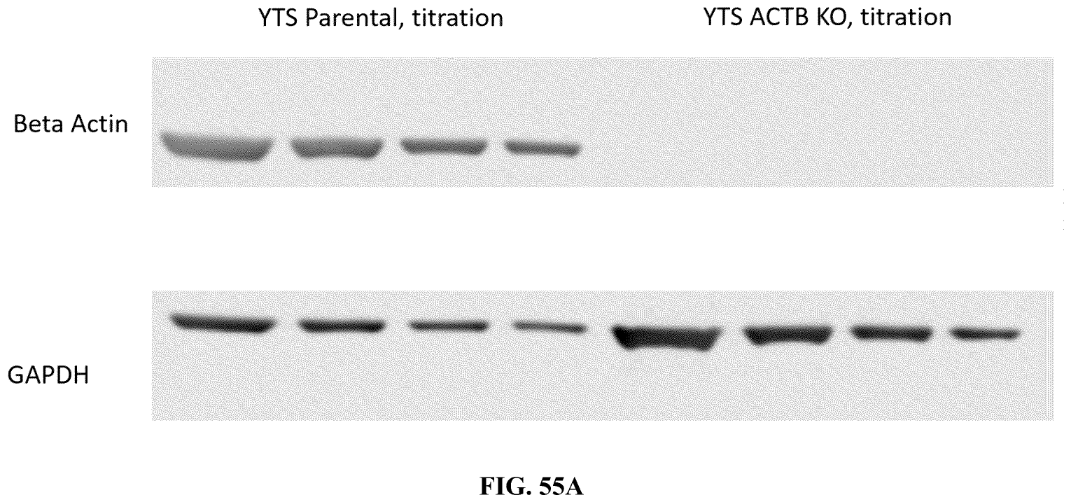

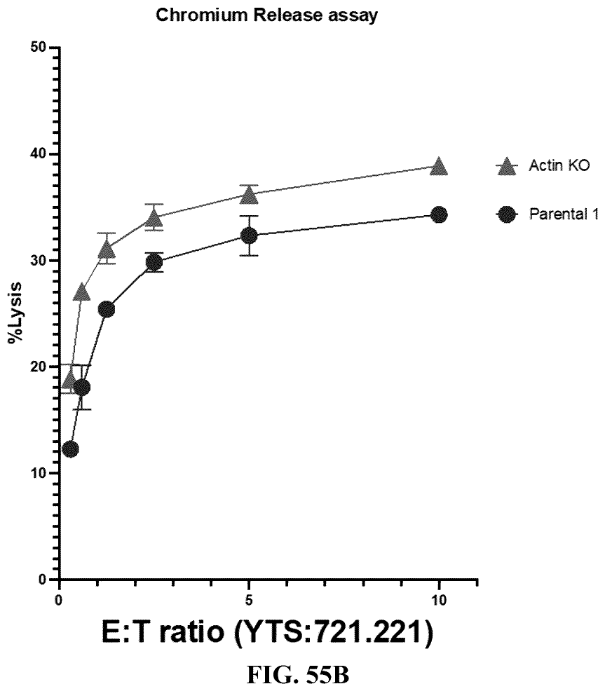

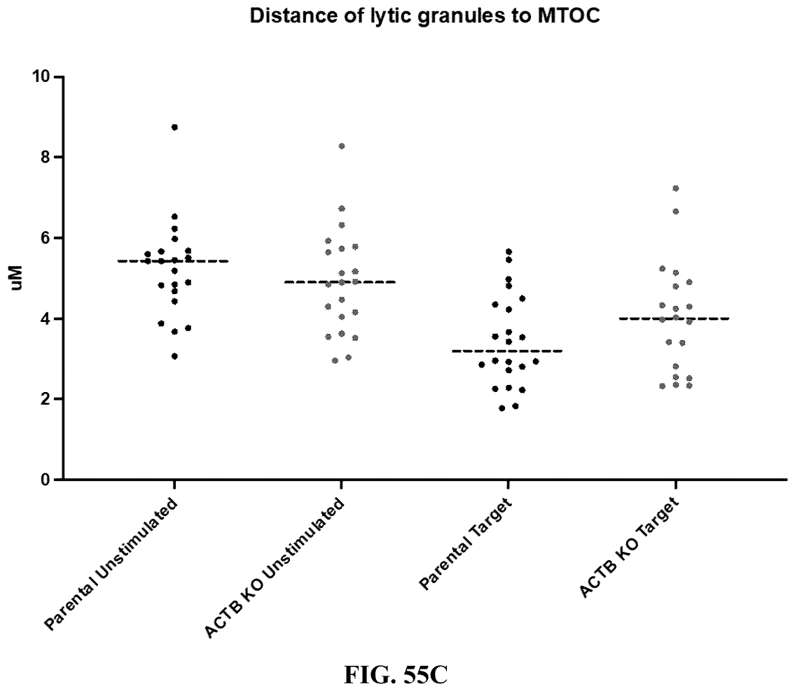

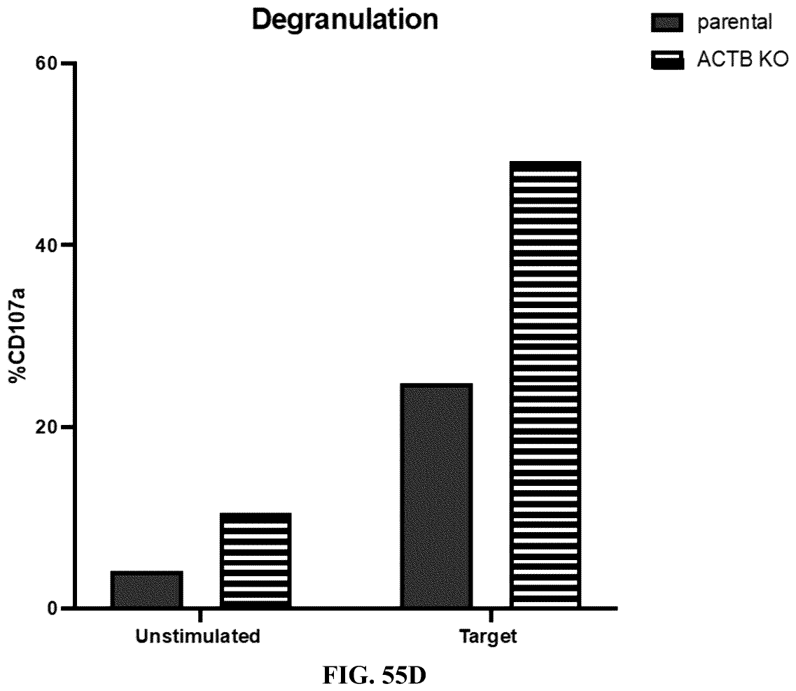

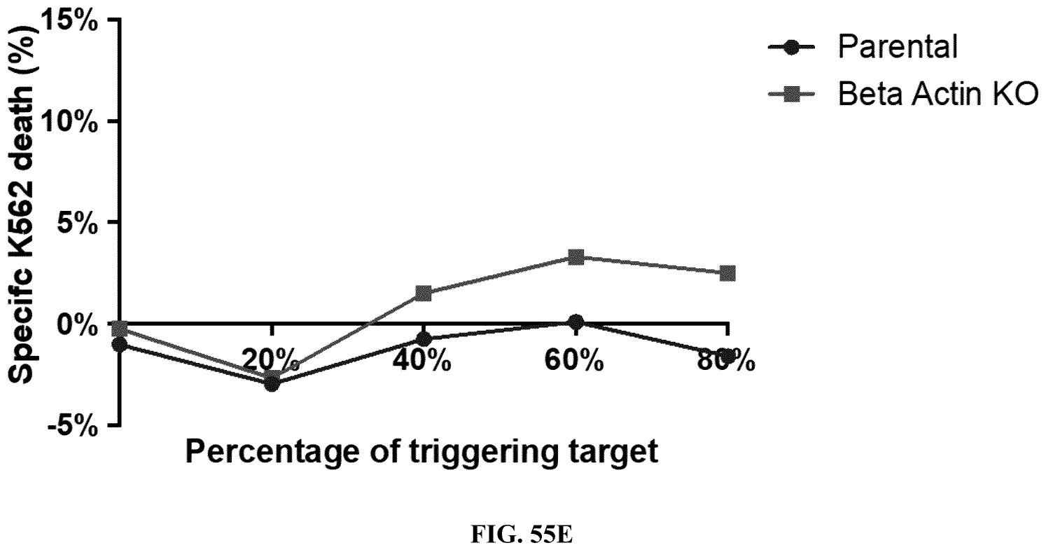

In an expansion of single clone with expression of RABGGTA knocked out using CRISPR/Cas9 (FIG. 54A), killing capacity of such RABGGTA KO NK cells is reduced though retained, unlike the bulk population of cells of RABGGTA KD NK cells (FIG. 54B). Specifically, YTS cells depleted for RABGGTA show an increased distance of the granules to the mTOC on activating surface (CD28) compared to parental cell line, suggesting an increase in granule dispersion in clone B1 cells (FIG. 54C). The CD28 label in FIG. 54C refers to a glass surface coated with anti-CD28 antibody and is an activating condition. The CD18 label in FIG. 54C refers to a glass surface coated with anti-CD18 antibody and is the unstimulated condition. For the CD107a degranulation analysis, YTS cells were stimulated for 60 min with target cells 721.221, stained for CD107a, and analyzed by FACS. As shown in FIG. 54D, RABGGTA KO cells have increased level of degranulation when compared to YTS parental cells. TheCOS model created with YTS parental cells and RABGGTA KO cells to study the killing efficiency of bystander K562 cells showed that RABGGTA KO cells kill resistant bystander K562 cells (FIG. 54E).

### 4. Cytoskeleton-Related Proteins

a. HkRP3

HkRP3 is required for lytic granule convergence. HkRP3 was deleted from YTS cells via CRISPR/Cas9 gene editing. Western blot analysis confirmed the absence of HkRP3 protein in edited cells relative to a tubulin loading control (FIG. 17A).

Release of Cr51 from target cells when co incubated with NK cells (cell lines or ex vivo cells) is the gold standard for assessing NK cell function. The higher CPM of ⁵¹Cr indicate the more efficient killing, for this, NK92 cells were incubated with targets K562 (previously marked with ⁵¹Cr) in different ratios (as showed on the FIG. 43A), during 4 hours. The supernatant was transferred to LUMA plates and read on a gamma counter, for the CPM. HkRP3-deleted YTS cells lysed ⁵¹Cr-labeled 721.221 target less efficiently in a 4-hour assay (FIG. 17B). YTS HKRP3 KO cells were incubated with targets 721.221 for 60 min and stained for perforin (granules marker) and tubulin, to track the MTOC. The average distance between the granules and the MTOC was measured on the software Imaris. Exemplary confocal images of control and HkRP3-deleted YTS cells (top cells) conjugated with 721.221 target cells (bottom cells) fixed and stained for perforin, a-tubulin, and DNA are shown in FIG. 17C. Lytic granules are converged in control cells and dispersed in the HkRP3-deleted YTS cells. The MTOC is marked by a white star. FIG. 17D shows quantification of lytic granule convergence. MGD was measured for 721.211-conjugated control and HkRP3-deleted YTS cells (*=p<0.01).

b. Septins

Septins are GTP binding proteins. Septins bind other septins to from heterooligomeric complexes such as filaments of the septin cytoskeleton and can also form septin cages or septin ring structures. Septins are involved in many biological processes, such as actin dynamics, microtubule regulation, cell shape and rigidity, chromosome segregation, cytokinesis, DNA repair, bacterial clearance, membrane trafficking, and neurotransmitter release.

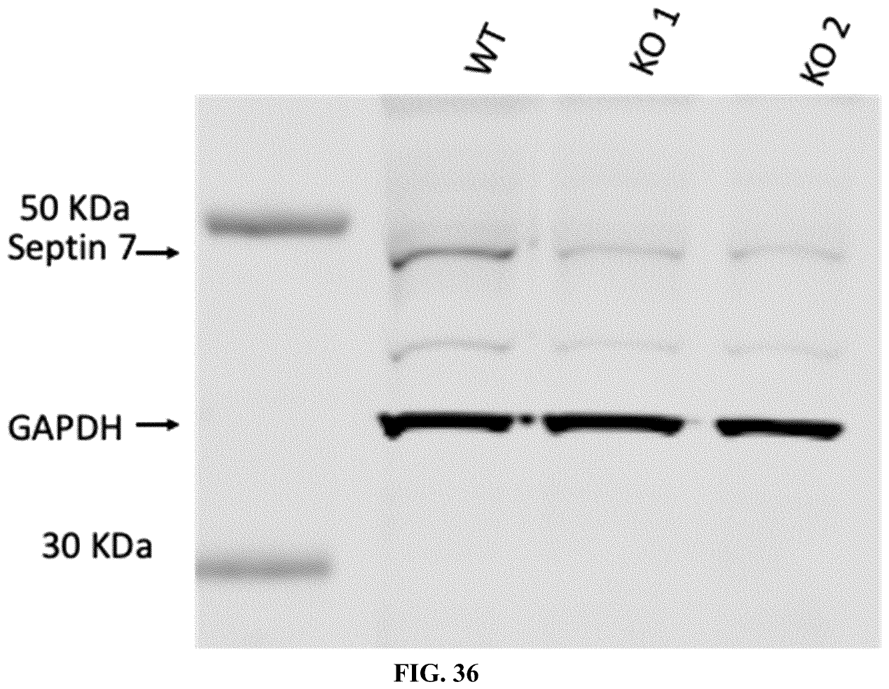

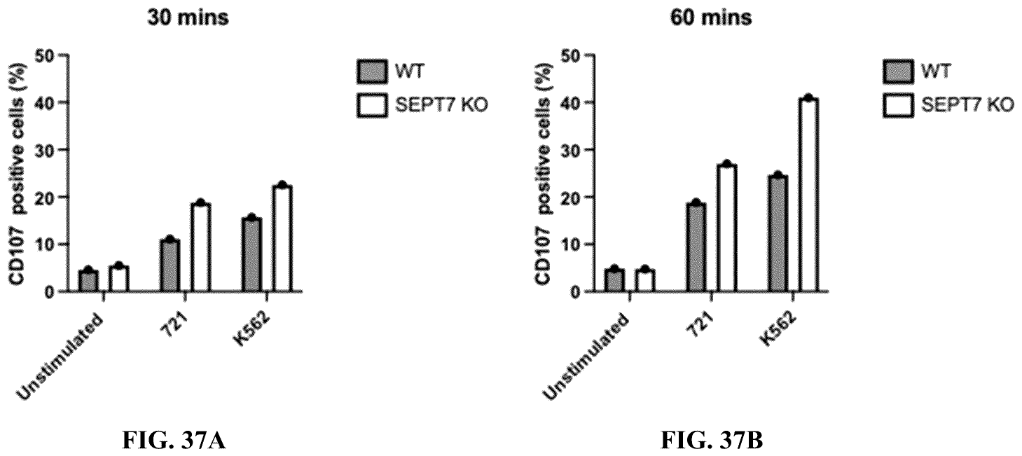

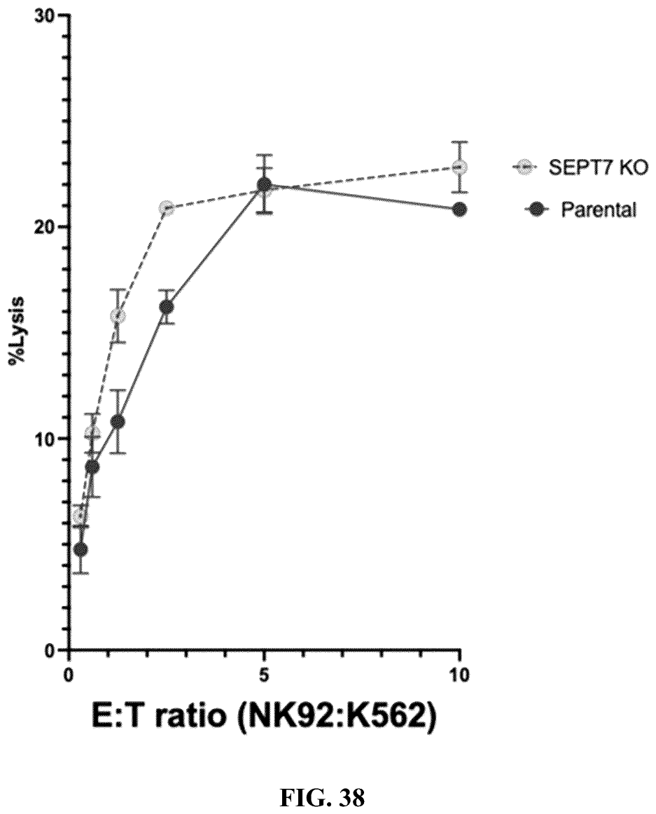

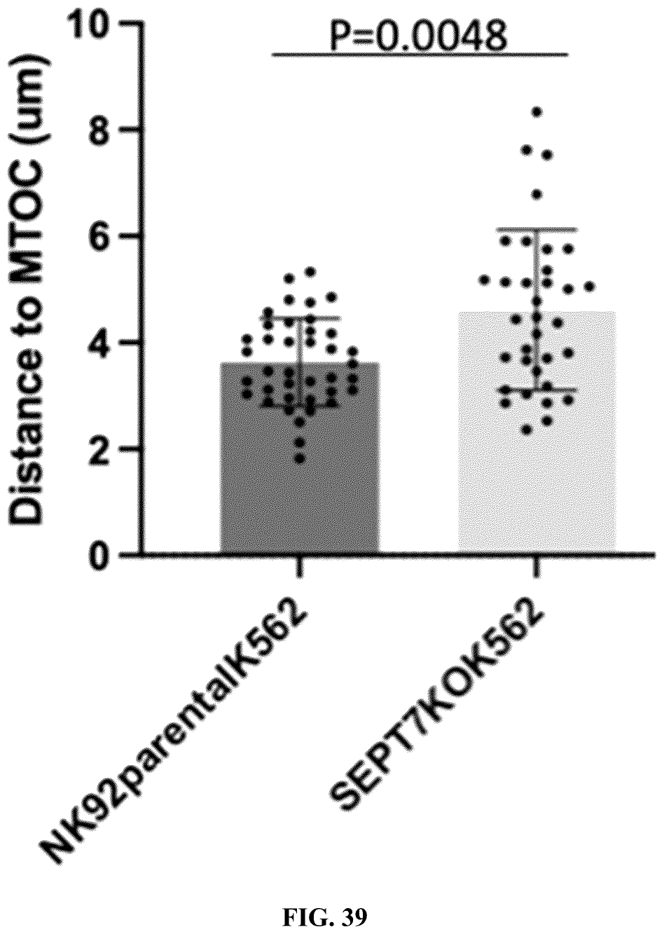

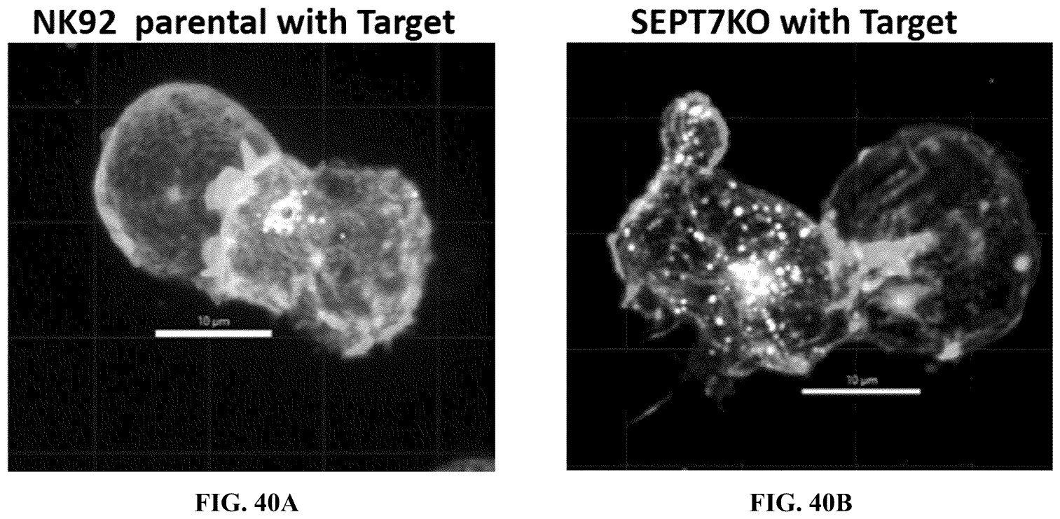

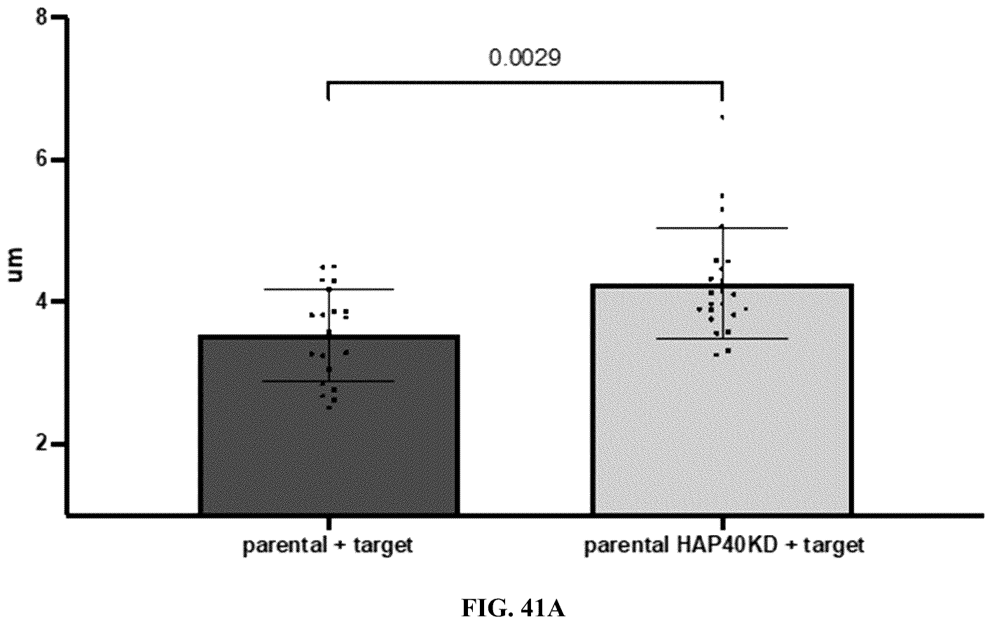

Septin-7 is the only indispensable protein for filament formation. Septin-7 co-localizes with lytic granules. SEPTIN-7 gene was knocked out using CRISPR Edition with transient CAS9 transfection in NK92 cell lines (FIG. 36). NK92 Septin-7 KO cells were incubated with targets K562 for 60 min and stained for perforin (granules marker) and tubulin, to track the MTOC. The average distance between the granules and the mTOC was measured using the software Imaris. NK92 Septin-7 KO cells were stimulated for 30 min (FIG. 37A) or 60 min (FIG. 37B) with 721.221 target cells or K562 target cells and then stained for CD107a and analyzed by FACS. For both periods of stimulation, Septin-7-deficient cells showed an increase in degranulation after conjugation with 721.221 target cells and K562 target cells. The killing efficiency of the NK92 cells KO for Septin-7 is retained, as shown in the Cr51 assay (FIG. 38). Septin-7-deficient NK92 cells show increase in the distance of the granules to the MTOC during the conjugation with Target cell, suggesting an increase on granule dispersion (FIGS. 39, 40A, and 40B).

c. HAP40

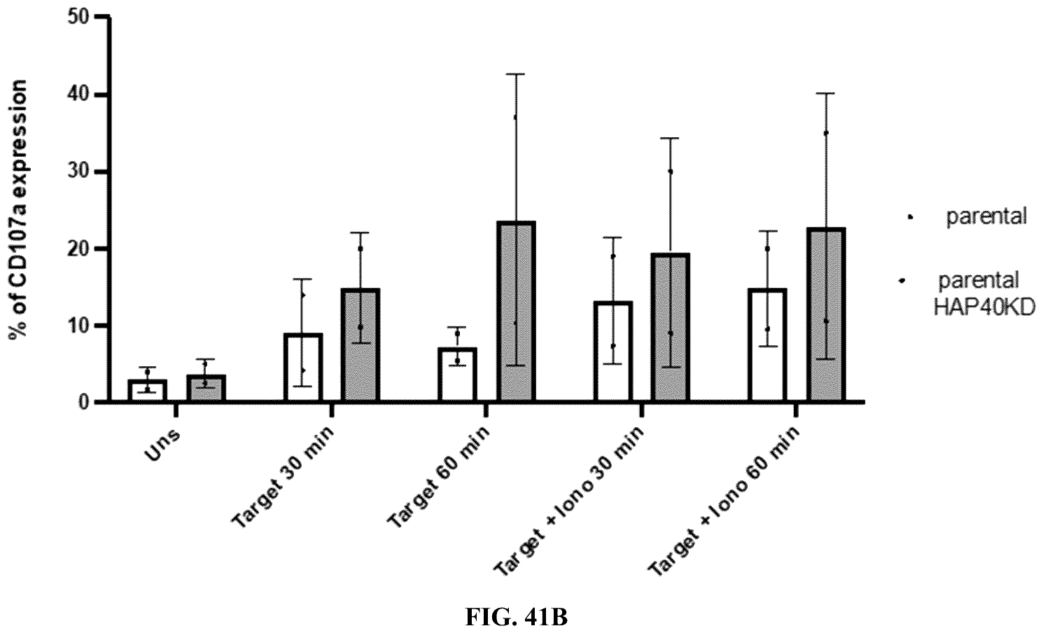

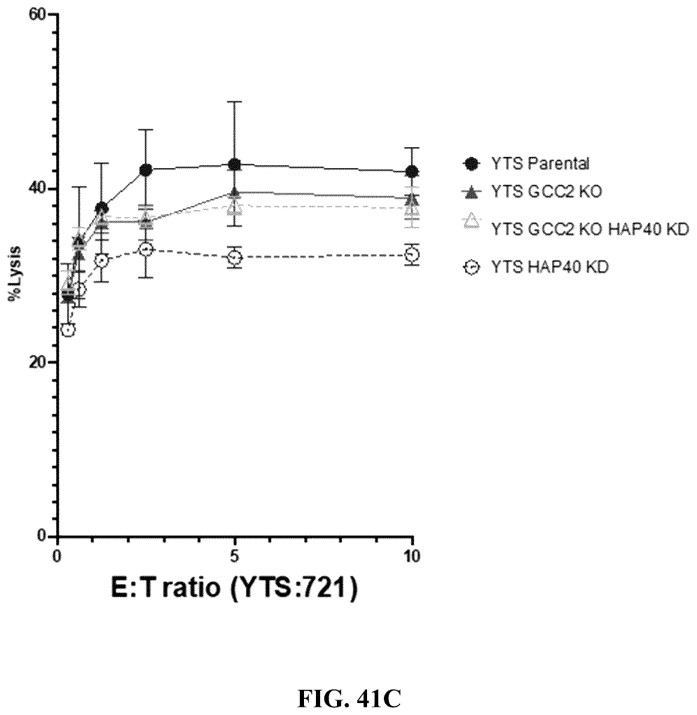

HAP40 is an adapter protein important for the transfer of vesicles from tubulin to the actin network and is highly expressed on NK cells. HAP40 is also predicted to interact with GCC2, based on mass spectra data. Thus, HAP40 could be one of the adapters of GCC2 with the granules and thus have similar function (FIGS. 41A-41C). YTS cells were transduced with lentivrus containing shRNA specific for HAP40 and kept in culture under puromycin selection. YTS HAP40 KD cells were incubated with 721.221 target cells for 60 minutes and stained for perforin (granules marker) and tubulin (to track the mTOC). The average distance between the granules and the mTOC was measured using Imaris. YTS HAP40 KD cells were stimulated for 60 min with target cells 721.221 and then stained for CD107a and analyzed by FACS.

d. CCDC84

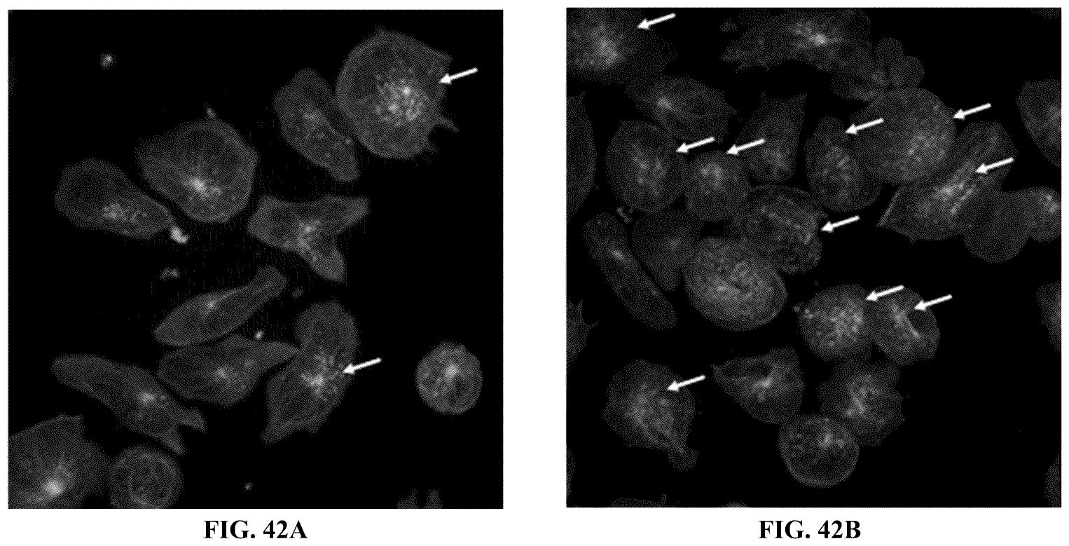

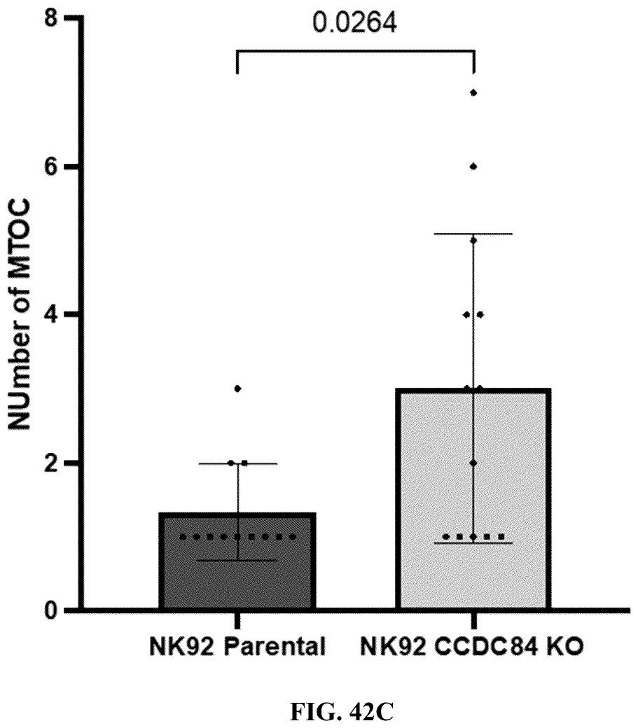

CCDC84 gene was knocked out using CRISPR Edition with transient CAS9 transfection in NK92 cell lines. The killing efficiency of the NK92 treated cells are within normal range, suggesting the NK92 cells are still efficient in killing target cells (FIGS. 42A-42C). The killing efficiency of the CCDC84 KO NK92 cells is conserved (FIG. 43A). The cells show increase in the distance of the granules to the MTOC during the conjugation with target cells, suggesting an increase on granule dispersion (FIG. 43B). Release of Cr51 from target cells when co incubated with NK cells (cell lines or ex vivo cells) is the gold standard for assessing NK cell function. The higher CPM of Cr51 indicate the more efficient killing, for this, NK92 cells were incubated with K562 target cells (previously marked with Cr51) in different ratios (as showed on the FIG. 43A), during 4 hours. The supernatant was transferred to LUMA plates and read on a gamma counter, for the CPM. NK92 CCDC84 KO cells were stimulated for 60 min with 721.221 target cells or K562 target cells and then stained for CD107a and analyzed by FACS. For the determination of MTOC, cells were fixed and stained for alpha-tubulin, where the hyper densities f alpha-tubulin are the MTOC.

e. ACTB (Beta Actin)

ACTB is one of six isomers of the actin protein. ACTB is a key protein for cytoskeletal filament formation in cells. Actin cytoskeleton regulates degranulation behavior of lytic granules in NK cells. Beta Actin was knocked out using CRISPR/Cas9 edition and a single clone was selected. The Western blot demonstrates that no Beta Actin protein is measurable in the ACTB KO cells compared to YTS parental relative to GAPDH loading control (FIG. 54A). The killing efficiency of YTS ACTB KO cells towards 721.221 target cells is increased compared to YTS parental NK cells indicating that ACTB KO cells are very efficient killers of target cells (FIG. 54B). YTS cells deficient for ACTB show an increasing trend in the distance of the granules to the mTOC during the conjugation with target cells Indicating increased granule dispersion for YTS ACTB KO cells (FIG. 54C). YTS cells were stimulated for 60 min with target cells 721.221, stained for CD107a and analyzed by FACS. ACTB KO cells and show an increase in the level of degranulation when compared to YTS parental cells (FIG. 54D). TheCOS model created with YTS parental cells and ACTB KO cells to study the killing efficiency of bystander K562 cells show that ACTB KO cells kill resistant bystander K562 cells (FIG. 54E).

It is to be understood that the present subject matter is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the technology as defined by the appended claims.

### REFERENCES

1. Adamo A, Delfino P, Gatti A, Bonato A, Takam Kamga P, Bazzoni R, Ugel S, Mercuri A, Caligola S, Krampera M. HS-5 and HS-27A Stromal Cell Lines to Study Bone Marrow Mesenchymal Stromal Cell-Mediated Support to Cancer Development. Front Cell Dev Biol. 2020; 8:584232. Epub 2020 Dec. 1. doi: 10.3389/fcell.2020.584232. PubMed PMID: 33251214; PMCID: PMC7674674.
2. Ahmed N, Brawley V S, Hegde M, Robertson C, Ghazi A, Gerken C, Liu E, Dakhova O, Ashoori A, Corder A, Gray T, Wu M F, Liu H, Hicks J, Rainusso N, Dotti G, Mei Z, Grilley B, Gee A, Rooney C M, Brenner M K, Heslop H E, Wels W S, Wang L L, Anderson P, Gottschalk S. Human Epidermal Growth Factor Receptor 2 (HER2)-Specific Chimeric Antigen Receptor-Modified T Cells for the Immunotherapy of HER2-Positive Sarcoma. J Clin Oncol. 2015;33 (15): 1688-96. Epub 2015 Mar. 25. doi: 10.1200/JCO.2014.58.0225. PubMed PMID: 25800760; PMCID: PMC4429176.
3. Ahmed N, Salsman V S, Yvon E, Louis C U, Perlaky L, Wels W S, Dishop M K, Kleinerman E E, Pule M, Rooney C M, Heslop H E, Gottschalk S. Immunotherapy for osteosarcoma: genetic modification of T cells overcomes low levels of tumor antigen expression. Mol Ther. 2009; 17 (10): 1779-87. Epub 2009 Jun. 18. doi: 10.1038/mt.2009.133. PubMed PMID: 19532139; PMCID: PMC2835000.
4. Anderson N M, Simon M C. The tumor microenvironment. Curr Biol. 2020; 30 (16): R921-R5. Epub 2020 Aug. 19. doi: 10.1016/j.cub.2020.06.081. PubMed PMID: 32810447; PMCID: PMC8194051.
5. Angelo L S, Banerjee P P, Monaco-Shawver L, Rosen J B, Makedonas G, Forbes L R, Mace E M, Orange J S. Practical N K cell phenotyping and variability in healthy adults. Immunol Res. 2015;62 (3): 341-56. Epub 2015 May 28. doi: 10.1007/s12026-015-8664-y. PubMed PMID: 26013798; PMCID: PMC4470870.
6. Banerjee P P, Orange J S. Quantitative measurement of F-actin accumulation at the N K cell immunological synapse. J Immunol Methods. 2010; 355 (1-2): 1-13. Epub 2010 Feb. 23. doi: 10.1016/j.jim.2010.02.003. PubMed PMID: 20171970; PMCID: PMC2854315.
7. Banerjee P P, Pandey R, Zheng R, Suhoski M M, Monaco-Shawver L, Orange J S. Cdc42-interacting protein-4 functionally links actin and microtubule networks at the cytolytic N K cell immunological synapse. J Exp Med. 2007; 204 (10): 2305-20. Epub 2007 Sep. 6. doi: 10.1084/jem.20061893. PubMed PMID: 17785506; PMCID: PMC2118451.
8. Banerjee P P, Pang L, Soldan S S, Miah S M, Eisenberg A, Maru S, Waldman A, Smith E A, Rosenberg-Hasson Y, Hirschberg D, Smith A, Ablashi D V, Campbell K S, Orange J S. KIR2DL4-HLAG interaction at human N K cell-oligodendrocyte interfaces regulates IFN-gamma-mediated effects. Mol Immunol. 2019; 115:39-55. Epub 2018 Nov. 30. doi: 10.1016/j.molimm.2018.09.027. PubMed PMID: 30482463; PMCID: PMC6543535.
9. Batista Leite S, Cipriano M, Carpi D, Coecke S, Holloway M, Corvi R, Worth A, Barroso J, Whelan M. Establishing the scientific validity of complex in vitro models: Results of a EURL ECVAM survey. Luxembourg; 2021.
10. Brudno J N, Kochenderfer J N. Recent advances in CAR T-cell toxicity: Mechanisms, manifestations and management. Blood Rev. 2019; 34:45-55. Epub 2018 Dec. 12. doi: 10.1016/j.blre.2018.11.002. PubMed PMID: 30528964; PMCID: PMC6628697.
11. Bryceson Y T, March M E, Barber D F, Ljunggren H G, Long E O. Cytolytic granule polarization and degranulation controlled by different receptors in resting N K cells. J Exp Med. 2005;202 (7): 1001-12. Epub 2005 Oct. 6. doi: 10.1084/jem.20051143. PubMed PMID: 16203869; PMCID: PMC2213171.
12. Bussard K M, Mutkus L, Stumpf K, Gomez-Manzano C, Marini F C. Tumor-associated stromal cells as key contributors to the tumor microenvironment. Breast Cancer Res. 2016; 18 (1): 84. Epub 2016 Aug. 16. doi: 10.1186/s13058-016-0740-2. PubMed PMID: 27515302; PMCID: PMC4982339.
13. Calaminus G, Jenney M, Hjorth L, Baust K, Bernstein M, Bielack S, Vos P, Hogendoorn P C W, Jovic G, Krailo M, Kreitz K, Marina N, Popoola B O, Sauerland C, Smeland S, Teske C, Schweinitz C V, Whelan J, Wiener A, Sydes M R, Nagarajan R. Quality of Life of Patients With Osteosarcoma in the European American Osteosarcoma Study-1 (EURAMOS-1): Development and Implementation of a Questionnaire Substudy. JMIR Res Protoc. 2019; 8 (8): e14406. Epub 2019 Oct. 2. doi: 10.2196/14406. PubMed PMID: 31573951; PMCID: PMC6819132.
14. Carisey A F, Mace E M, Saeed M B, Davis D M, Orange J S. Nanoscale Dynamism of Actin Enables Secretory Function in Cytolytic Cells. Curr Biol. 2018; 28 (4): 489-502 e9. Epub 2018 Feb. 6. doi: 10.1016/j.cub.2017.12.044. PubMed PMID: 29398219; PMCID: PMC5835143.
15. Chockley P, Patil S L, Gottschalk S. Transient blockade of TBK1/IKKepsilon allows efficient transduction of primary human natural killer cells with vesicular stomatitis virus G-pseudotyped lentiviral vectors. Cytotherapy. 2021; 23 (9): 787-92. Epub 2021 Jun. 14. doi: 10.1016/j.jcyt.2021.04.010. PubMed PMID: 34119434; PMCID: PMC8425283.
16. Comerci C J, Mace E M, Banerjee P P, Orange J S. CD2 promotes human natural killer cell membrane nanotube formation. PLOS One. 2012; 7 (10): e47664. Epub 2012 Nov. 1. doi: 10.1371/journal.pone.0047664. PubMed PMID: 23112830; PMCID: PMC3480409.
17. Culley F J, Johnson M, Evans J H, Kumar S, Crilly R, Casasbuenas J, Schnyder T, Mehrabi M, Deonarain M P, Ushakov D S, Braud V, Roth G, Brock R, Kohler K, Davis D M. Natural killer cell signal integration balances synapse symmetry and migration. PLOS Biol. 2009; 7 (7): e1000159. Epub 2009 Jul. 29. doi: 10.1371/journal.pbio. 1000159. PubMed PMID: 19636352; PMCID: PMC2707003.
18. DeRenzo C, Gottschalk S. Genetically Modified T-Cell Therapy for Osteosarcoma: Into the Roaring 2020s. Adv Exp Med Biol. 2020; 1257:109-31. Epub 2020 Jun. 3. doi: 10.1007/978-3-030-43032-0_10. PubMed PMID: 32483735; PMCID: PMC7385999.
19. Friedman D, Simmonds P, Hale A, Bere L, Hodson N W, White M R H, Davis D M. Natural killer cell immune synapse formation and cytotoxicity are controlled by tension of the target interface. J Cell Sci. 2021; 134 (7). Epub 2021 Mar. 14. doi: 10.1242/jcs.258570. PubMed PMID: 33712452; PMCID: PMC8077183.
20. Dolat, Lee, Qicong Hu, and Elias T. Spiliotis. “Septin functions in organ system physiology and pathology.” Biological chemistry 395.2 (2014): 123-141.
21. Goulding D, Arguinchona L, Anderson-Mellies A, Mikkelsen M, Eguchi M, Marinoff H, Zahedi S, Ribeiro K B, Cockburn M, Rodriguez Galindo C, Green A L. Sociodemographic Disparities in Presentation and Survival of Pediatric Bone Cancers. J Pediatr Hematol Oncol. 2022. Epub 2022 Sep. 1. doi:

10.1097/MPH.0000000000002531. PubMed PMID: 36044295.
22. Grier J T, Forbes L R, Monaco-Shawver L, Oshinsky J, Atkinson T P, Moody C, Pandey R, Campbell K S, Orange J S. Human immunodeficiency-causing mutation defines CD16 in spontaneous N K cell cytotoxicity. J Clin Invest. 2012; 122 (10): 3769-80. Epub 2012 Sep. 26. doi: 10.1172/JCI64837. PubMed PMID: 23006327; PMCID: PMC3461929.
23. Gunesch J T, Angelo L S, Mahapatra S, Deering R P, Kowalko J E, Sleiman P, Tobias J W, Monaco-Shawver L, Orange J S, Mace E M. Genome-wide analyses and functional profiling of human N K cell lines. Mol Immunol. 2019; 115:64-75. Epub 2018 Jul. 29. doi: 10.1016/j.molimm.2018.07.015. PubMed PMID: 30054012; PMCID: PMC6345623.
24. Gwalani L A, Orange J S. Single Degranulations in N K Cells Can Mediate Target Cell Killing. J Immunol. 2018; 200 (9): 3231-43. Epub 2018 Mar. 30. doi: 10.4049/jimmunol. 1701500. PubMed PMID: 29592963; PMCID: PMC6020067.
25. Halperin A, Kröger M, Winnik F M. Poly(N-isopropylacrylamide) Phase Diagrams: Fifty Years of Research. Angew Chem Int Ed Engl. 2015 Dec. 14;54 (51): 15342-67.
26. Ham H, Huynh W, Schoon R A, Vale R D, Billadeau D D. HKRP3 is a microtubule-binding protein regulating lytic granule clustering and N K cell killing. J Immunol. 2015;194 (8): 3984-96. Epub 2015 Mar. 13. doi: 10.4049/jimmunol.1402897. PubMed PMID: 25762780; PMCID: PMC4390494.
27. Hoing S, Yeh T Y, Baumann M, Martinez N E, Habenberger P, Kremer L, Drexler H C A, Kuchler P, Reinhardt P, Choidas A, Zischinsky M L, Zischinsky G, Nandini S, Ledray A P, Ketcham S A, Reinhardt L, Abo-Rady M, Glatza M, King S J, Nussbaumer P, Ziegler S, Klebl B, Schroer T A, Scholer H R, Waldmann H, Sterneckert J. Dynarrestin, a Novel Inhibitor of Cytoplasmic Dynein. Cell Chem Biol. 2018; 25 (4): 357-69 e6. Epub 2018 Feb. 6. doi: 10.1016/j.chembiol.2017.12.014. PubMed PMID: 29396292; PMCID: PMC8543760.
28. Hsu H T, Carisey A F, Orange J S. Measurement of Lytic Granule Convergence After Formation of an N K Cell Immunological Synapse. Methods Mol Biol. 2017; 1584:497-515. Epub 2017 Mar. 4. doi: 10.1007/978-1-4939-6881-7_31. PubMed PMID: 28255722; PMCID: PMC5861262.
29. Hsu H T, Mace E M, Carisey A F, Viswanath D I, Christakou A E, Wiklund M, Onfelt B, Orange J S. N K cells converge lytic granules to promote cytotoxicity and prevent bystander killing. J Cell Biol. 2016; 215 (6): 875-89. Epub 2016 Dec. 3. doi: 10.1083/jcb.201604136. PubMed PMID: 27903610; PMCID: PMC5166499.
30. Hsu K, Middlemiss S, Saletta F, Gottschalk S, McCowage G B, Kramer B. Chimeric Antigen Receptor-modified T cells targeting EphA2 for the immunotherapy of paediatric bone tumours. Cancer Gene Ther. 2021; 28 (3-4): 321-34. Epub 2020 Sep. 3. doi: 10.1038/s41417-020-00221-4. PubMed PMID: 32873870; PMCID: PMC8057949.
31. James A M, Hsu H T, Dongre P, Uzel G, Mace E M, Banerjee P P, Orange J S. Rapid activation receptor- or IL-2-induced lytic granule convergence in human natural killer cells requires Src, but not downstream signaling. Blood. 2013; 121 (14): 2627-37. Epub 2013 Feb. 6. doi: 10.1182/blood-2012-06-437012. PubMed PMID: 23380740; PMCID: PMC3617630.
32. June C H, O'Connor R S, Kawalekar O U, Ghassemi S, Milone M C. CAR T cell immunotherapy for human cancer. Science. 2018; 359 (6382): 1361-5. Epub 2018 Mar. 24. doi: 10.1126/science.aar6711. PubMed PMID: 29567707.
33. Jyonouchi S, Gwafila B, Gwalani L A, Ahmad M, Moertel C, Holbert C, Kim J Y, Kobrinsky N, Roy-Ghanta S, Orange J S. Phase I trial of low-dose interleukin 2 therapy in patients with Wiskott-Aldrich syndrome. Clin Immunol. 2017; 179:47-53. Epub 2017 Feb. 25. doi: 10.1016/j.clim.2017.02.001. PubMed PMID: 28232030.
34. Kadomoto S, Izumi K, Hiratsuka K, Nakano T, Naito R, Makino T, Iwamoto H, Yaegashi H, Shigehara K, Kadono Y, Nakata H, Saito Y, Nakagawa-Goto K, Mizokami A. Tumor-Associated Macrophages Induce Migration of Renal Cell Carcinoma Cells via Activation of the CCL20-CCR6 Axis. Cancers (Basel). 2019; 12 (1). Epub 2020 Jan. 8. doi: 10.3390/cancers12010089. PubMed PMID: 31905918; PMCID: PMC7017081.
35. Kahn J M, Beauchemin M. Improving Health Equity and Reducing Disparities in Pediatric and Adolescent/Young Adult Oncology: In Support of Clinical Practice Guidelines. J Natl Compr Canc Netw. 2021; 19 (6): 765-9. Epub 2021 Jul. 3. doi: 10.6004/jnccn.2021.7048. PubMed PMID: 34214972.
36. Kritikou J S, Oliveira M M, Record J, Saeed M B, Nigam S M, He M, Keszei M, Wagner A K, Brauner H, Sendel A, Sedimbi S K, Rentouli S, Lane D P, Snapper S B, Karre K, Vandenberghe P, Orange J S, Westerberg L S. Constitutive activation of WASp leads to abnormal cytotoxic cells with increased granzyme B and degranulation response to target cells. JCI Insight. 2021; 6 (6). Epub 2021 Feb. 24. doi: 10.1172/jci.insight.140273. PubMed PMID: 33621210; PMCID: PMC8026198.
37. Labani-Motlagh A, Ashja-Mahdavi M, Loskog A. The Tumor Microenvironment: A Milieu Hindering and Obstructing Antitumor Immune Responses. Front Immunol. 2020; 11:940. Epub 2020 Jun. 6. doi: 10.3389/fimmu.2020.00940. PubMed PMID: 32499786; PMCID: PMC7243284.
38. Lafleur E A, Koshkina N V, Stewart J, Jia S F, Worth L L, Duan X, Kleinerman E S. Increased Fas expression reduces the metastatic potential of human osteosarcoma cells. Clin Cancer Res. 2004; 10 (23): 8114-9. Epub 2004 Dec. 9. doi: 10.1158/1078-0432.CCR-04-0353. PubMed PMID: 15585647.
39. Lam M T, Mace E M, Orange J S. A research-driven approach to the identification of novel natural killer cell deficiencies affecting cytotoxic function. Blood. 2020; 135 (9): 629-37. Epub 2020 Jan. 17. doi: 10.1182/blood.2019000925. PubMed PMID: 31945148; PMCID: PMC7046607.
40. Lee D A. Cellular therapy: Adoptive immunotherapy with expanded natural killer cells. Immunol Rev. 2019; 290 (1): 85-99. Epub 2019 Jul. 30. doi: 10.1111/imr.12793. PubMed PMID: 31355489.
41. Li Y, Orange J S. Degranulation enhances presynaptic membrane packing, which protects perforin-mediated autolysis. PLOS Biol. 2021; 19 (8): e3001328. Epub 2021 Aug. 4. doi: 10.1371/journal.pbio.3001328. PubMed PMID: 34343168; PMCID: PMC8330931.
42. Lian G, Mak T S, Yu X, Lan H Y. Challenges and Recent Advances in N K Cell-Targeted Immunotherapies in Solid Tumors. Int J Mol Sci. 2021; 23 (1). Epub 2022 Jan. 12. doi: 10.3390/ijms23010164. PubMed PMID: 35008589; PMCID: PMC8745474.
43. Mace E M, Bigley V, Gunesch J T, Chinn I K, Angelo L S, Care M A, Maisuria S, Keller M D, Togi S, Watkin L B, LaRosa D F, Jhangiani S N, Muzny D M, Stray-Pedersen A, Coban Akdemir Z, Smith J B, Hernandez-Sanabria M, Le D T, Hogg G D, Cao T N, Freud A G, Szymanski E P, Savic S, Collin M, Cant A J, Gibbs R A, Holland S M, Caligiuri M A, Ozato K, Paust S, Doody G M, Lupski J R, Orange J S. Biallelic mutations in IRF8 impair human N K cell maturation and function. J Clin Invest. 2017;127 (1): 306-20. Epub 2016 Nov. 29. doi: 10.1172/JCI86276. PubMed PMID: 27893462; PMCID: PMC5199714.
44. Mace E M, Dongre P, Hsu H T, Sinha P, James A M, Mann S S, Forbes L R, Watkin L B, Orange J S. Cell biological steps and checkpoints in accessing N K cell cytotoxicity. Immunol Cell Biol. 2014; 92 (3): 245-55. Epub 2014 Jan. 22. doi: 10.1038/icb.2013.96. PubMed PMID: 24445602; PMCID: PMC3960583.
45. Mace E M, Gunesch J T, Dixon A, Orange J S. Human N K cell development requires CD56-mediated motility and formation of the developmental synapse. Nat Commun. 2016; 7:12171. Epub 2016 Jul. 21. doi: 10.1038/ncomms12171. PubMed PMID: 27435370; PMCID: PMC4961740.
46. Mace E M, Orange J S. Dual channel STED nanoscopy of lytic granules on actin filaments in natural killer cells. Commun Integr Biol. 2012; 5 (2): 184-6. Epub 2012 Jul. 19. doi: 10.4161/cib.18818. PubMed PMID: 22808328; PMCID: PMC3376059.
47. Mace E M, Orange J S. High- and Super-Resolution Microscopy Imaging of the N K Cell Immunological Synapse. Methods Mol Biol. 2016; 1441:141-50. Epub 2016 May 15. doi: 10.1007/978-1-4939-3684-7_12. PubMed PMID: 27177663.
48. Mace E M, Orange J S. Lytic immune synapse function requires filamentous actin deconstruction by Coronin 1A. Proc Natl Acad Sci USA. 2014; 111 (18): 6708-13. Epub 2014 Apr. 25. doi: 10.1073/pnas.1314975111. PubMed PMID: 24760828; PMCID: PMC4020046.
49. Mace E M, Orange J S. Visualization of the immunological synapse by dual color time-gated stimulated emission depletion (STED) nanoscopy. J Vis Exp. 2014 (85). Epub 2014 Apr. 2. doi: 10.3791/51100. PubMed PMID: 24686478; PMCID: PMC4157735.
50. Mace E M, Paust S, Conte M I, Baxley R M, Schmit M M, Patil S L, Guilz N C, Mukherjee M, Pezzi A E, Chmielowiec J, Tatineni S, Chinn I K, Akdemir Z C, Jhangiani S N, Muzny D M, Stray-Pedersen A, Bradley R E, Moody M, Connor P P, Heaps A G, Steward C, Banerjee P P, Gibbs R A, Borowiak M, Lupski J R, Jolles S, Bielinsky A K, Orange J S. Human N K cell deficiency as a result of biallelic mutations in MCM10. J Clin Invest. 2020; 130 (10): 5272-86. Epub 2020 Sep. 1. doi: 10.1172/JCI134966. PubMed PMID: 32865517; PMCID: PMC7524476.
51. Mace E M, Wu W W, Ho T, Mann S S, Hsu H T, Orange J S. N K cell lytic granules are highly motile at the immunological synapse and require F-actin for post-degranulation persistence. J Immunol. 2012; 189 (10): 4870-80. Epub 2012 Oct. 16. doi: 10.4049/jimmunol. 1201296. PubMed PMID: 23066148; PMCID: PMC3558996.
52. Mahapatra S, Mace E M, Minard C G, Forbes L R, Vargas-Hernandez A, Duryea T K, Makedonas G, Banerjee P P, Shearer W T, Orange J S. High-resolution phenotyping identifies N K cell subsets that distinguish healthy children from adults. PLOS One. 2017;12 (8): e0181134. Epub 2017 Aug. 3. doi: 10.1371/journal.pone.0181134. PubMed PMID: 28767726; PMCID: PMC5540415.
53. Majzner R G, Mackall C L. Tumor Antigen Escape from CAR T-cell Therapy. Cancer Discov. 2018; 8 (10): 1219-26. Epub 2018 Aug. 24. doi: 10.1158/2159-8290.CD-18-0442. PubMed PMID: 30135176.
54. Marofi F, Abdul-Rasheed O F, Rahman H S, Budi H S, Jalil A T, Yumashev A V, Hassanzadeh A, Yazdanifar M, Motavalli R, Chartrand M S, Ahmadi M, Cid-Arreguid A, Jarahian M. CAR-N K cell in cancer immunotherapy; A promising frontier. Cancer Sci. 2021; 112 (9): 3427-36 Epub 2021 May 30. doi: 10.1111/cas.14993. PubMed PMID: 34050690; PMCID: PMC8409419.
55. Mentlik A N, Sanborn K B, Holzbaur E L, Orange J S. Rapid lytic granule convergence to the MTOC in natural killer cells is dependent on dynein but not cytolytic commitment. Mol Biol Cell. 2010; 21 (13): 2241-56. Epub 2010 May 7. doi: 10.1091/mbc. E09-11-0930. PubMed PMID: 20444980; PMCID: PMC2893988.
56. Mizesko M C, Banerjee P P, Monaco-Shawver L, Mace E M, Bernal W E, Sawalle-Belohradsky J, Belohradsky B H, Heinz V, Freeman A F, Sullivan K E, Holland S M, Torgerson T R, Al-Herz W, Chou J, Hanson I C, Albert M H, Geha R S, Renner E D, Orange J S. Defective actin accumulation impairs human natural killer cell function in patients with dedicator of cytokinesis 8 deficiency. J Allergy Clin Immunol. 2013; 131 (3): 840-8. Epub 2013 Feb. 6. doi: 10.1016/j.jaci.2012.12.1568. PubMed PMID: 23380217; PMCID: PMC3646579.
57. Mohanty R, Chowdhury C R, Arega S, Sen P, Ganguly P, Ganguly N. CAR T cell therapy: A new era for cancer treatment (Review). Oncol Rep. 2019; 42 (6): 2183-95. Epub 2019 Oct. 4. doi: 10.3892/or.2019.7335. PubMed PMID: 31578576.
58. Monaco-Shawver L, Schwartz L, Tuluc F, Guo C J, Lai J P, Gunnam S M, Kilpatrick L E, Banerjee P P, Douglas S D, Orange J S. Substance P inhibits natural killer cell cytotoxicity through the neurokinin-1 receptor. J Leukoc Biol. 2011; 89 (1): 113-25. Epub 2010 Oct. 14. doi: 10.1189/jlb.0410200. PubMed PMID: 20940324; PMCID: PMC3004520.
59. Moore K J, Barragan F, Williams L A. Survival disparities for childhood cancers exist when defined by race/ethnicity and sex. Cancer Epidemiol. 2022; 81:102262. Epub 2022 Sep. 21. doi: 10.1016/j.canep.2022.102262. PubMed PMID: 36126471.
60. Mostowy, Serge, and Pascale Cossart. “Septins: the fourth component of the cytoskeleton.” Nature reviews Molecular cell biology 13.3 (2012): 183-194.
61. Mukherjee M, Mace E M, Carisey A F, Ahmed N, Orange J S. Quantitative Imaging Approaches to Study the CAR Immunological Synapse. Mol Ther. 2017;25 (8): 1757-68. Epub 2017 Jul. 1. doi: 10.1016/j.ymthe.2017.06.003. PubMed PMID: 28663103; PMCID: PMC5542801.
62. Nakamura K, Smyth M J. Myeloid immunosuppression and immune checkpoints in the tumor microenvironment. Cell Mol Immunol. 2020; 17 (1): 1-12. Epub 2019 Oct. 16. doi: 10.1038/s41423-019-0306-1. PubMed PMID: 31611651; PMCID: PMC6952382.
63. Nguyen P, Okeke E, Clay M, Haydar D, Justice J, O'Reilly C, Pruett-Miller S, Papizan J, Moore J, Zhou S, Throm R, Krenciute G, Gottschalk S, DeRenzo C. Route of 41B B/41BBL Costimulation Determines Effector Function of B7-H3-CAR.CD28zeta T Cells. Mol Ther Oncolytics. 2020; 18:202-14. Epub 2020 Jul. 31. doi: 10.1016/j.omto.2020.06.018. PubMed PMID: 32728609; PMCID: PMC7369352.
64. Orange J S, Roy-Ghanta S, Mace E M, Maru S, Rak G D, Sanborn K B, Fasth A, Saltzman R, Paisley A, Monaco-Shawver L, Banerjee P P, Pandey R. IL-2 induces a WAVE2-dependent pathway for actin reorganization that enables WASp-independent human N K cell function. J Clin Invest. 2011; 121 (4): 1535-48. Epub 2011 Mar. 9. doi: 10.1172/JCI44862. PubMed PMID: 21383498; PMCID: PMC3069781.
65. Orange J S. Formation and function of the lytic N K-cell immunological synapse. Nat Rev Immunol. 2008;8 (9): 713-25. Epub 2009 Jan. 28. doi: 10.1038/nri2381. PubMed PMID: 19172692; PMCID: PMC2772177.
66. Orange J S. The lytic N K cell immunological synapse and sequential steps in its formation. Adv Exp Med Biol. 2007; 601:225-33. Epub 2007 Aug. 24. doi: 10.1007/978-0-387-72005-O_23. PubMed PMID: 17713009.
67. Pandey R, DeStephan C M, Madge L A, May M J, Orange J S. NKp30 ligation induces rapid activation of the canonical N F-kappaB pathway in N K cells. J Immunol. 2007; 179 (11): 7385-96. Epub 2007 Nov. 21. doi: 10.4049/jimmunol.179.11.7385. PubMed PMID: 18025182; PMCID: PMC4349331.
68. Phatarpekar P V, Billadeau D D. Molecular regulation of the plasma membrane-proximal cellular steps involved in N K cell cytolytic function. J Cell Sci. 2020; 133 (5). Epub 2020 Feb. 23. doi: 10.1242/jcs.240424. PubMed PMID: 32086255; PMCID: PMC7044456.
69. Phatarpekar, P. V., Overlee, B. L., Leehan, A., Wilton, K. M., Ham, H., & Billadeau, D. D. (2020). The septin cytoskeleton regulates natural killer cell lytic granule release. Journal of Cell Biology, 219 (11).
70. Poggi A, Villa F, Fernadez J L C, Costa D, Zocchi M R, Benelli R. Three-Dimensional Culture Models to Study Innate Anti-Tumor Immune Response: Advantages and Disadvantages. Cancers (Basel). 2021; 13 (14). Epub 2021 Jul. 25. doi: 10.3390/cancers13143417. PubMed PMID: 34298630; PMCID: PMC8303518.
71. Prager I, Watzl C. Mechanisms of natural killer cell-mediated cellular cytotoxicity. J Leukoc Biol. 2019; 105 (6): 1319-29. Epub 2019 May 21. doi: 10.1002/JLB.MR0718-269R. PubMed PMID: 31107565.
72. Prinzing B, Zebley C C, Petersen C T, Fan Y, Anido A A, Yi Z, Nguyen P, Houke H, Bell M, Haydar D, Brown C, Boi S K, Alli S, Crawford J C, Riberdy J M, Park J J, Zhou S, Velasquez M P, DeRenzo C, Lazzarotto C R, Tsai S Q, Vogel P, Pruett-Miller S M, Langfitt D M, Gottschalk S, Youngblood B, Krenciute G. Deleting DNMT3A in CAR T cells prevents exhaustion and enhances antitumor activity. Sci Transl Med. 2021; 13 (620): eabh0272. Epub 2021 Nov. 18. doi: 10.1126/scitranslmed.abh0272. PubMed PMID: 34788079; PMCID: PMC8733956.
73. Rainusso N, Brawley V S, Ghazi A, Hicks M J, Gottschalk S, Rosen J M, Ahmed N. Immunotherapy targeting HER2 with genetically modified T cells eliminates tumor-initiating cells in osteosarcoma. Cancer Gene Ther. 2012; 19 (3): 212-7. Epub 2011 Dec. 17. doi: 10.1038/cgt.2011.83. PubMed PMID: 22173710.
74. Rak G D, Mace E M, Banerjee P P, Svitkina T, Orange J S. Natural killer cell lytic granule secretion occurs through a pervasive actin network at the immune synapse. PLOS Biol. 2011; 9 (9): e1001151. Epub 2011 Sep. 21. doi: 10.1371/journal.pbio. 1001151. PubMed PMID: 21931536; PMCID: PMC3172191.
75. Reed A E, Mace E M. A WICB 50th Favorite: Rapid lytic granule convergence to the MTOC in natural killer cells is dependent on dynein but not cytolytic commitment. Mol Biol Cell. 2022;33 (2): fe2. Epub 2022 Jan. 19. doi: 10.1091/mbc.E21-04-0218. PubMed PMID: 35041469.
76. Ritter A T, Mellman I. Natural killer cell granules converge to avoid collateral damage. J Cell Biol. 2016; 215 (6): 765-7. Epub 2016 Dec. 10. doi: 10.1083/jcb.201612003. PubMed PMID: 27932575; PMCID: PMC5166509.
77. Salzer E, Cagdas D, Hons M, Mace E M, Garncarz W, Petronczki O Y, Platzer R, Pfajfer L, Bilic I, Ban S A, Willmann K L, Mukherjee M, Supper V, Hsu H T, Banerjee P P, Sinha P, McClanahan F, Zlabinger G J, Pickl W F, Gribben J G, Stockinger H, Bennett K L, Huppa J B, Dupre L, Sanal O, Jager U, Sixt M, Tezcan I, Orange J S, Boztug K. RASGRP1 deficiency causes immunodeficiency with impaired cytoskeletal dynamics. Nat Immunol. 2016; 17 (12): 1352-60. Epub 2016 Nov. 1. doi: 10.1038/ni.3575. PubMed PMID: 27776107; PMCID: PMC6400263.
78. Sanborn K B, Mace E M, Rak G D, Difeo A, Martignetti J A, Pecci A, Bussel J B, Favier R, Orange J S. Phosphorylation of the myosin IIA tailpiece regulates single myosin IIA molecule association with lytic granules to promote N K-cell cytotoxicity. Blood. 2011; 118 (22): 5862-71. Epub 2011 Nov. 30. doi: 10.1182/blood-2011-03-344846. PubMed PMID: 22123909; PMCID: PMC3228501.
79. Sanborn K B, Orange J S. Navigating barriers: the challenge of directed secretion at the natural killer cell lytic immunological synapse. J Clin Immunol. 2010; 30 (3): 358-63. Epub 2010 Mar. 2. doi: 10.1007/s10875-010-9372-y. PubMed PMID: 20191315; PMCID: PMC2874632.
80. Sanborn K B, Rak G D, Maru S Y, Demers K, Difeo A, Martignetti J A, Betts M R, Favier R, Banerjee P P, Orange J S. Myosin IIA associates with N K cell lytic granules to enable their interaction with F-actin and function at the immunological synapse. J Immunol. 2009; 182 (11): 6969-84. Epub 2009 May 21. doi: 10.4049/jimmunol.0804337. PubMed PMID: 19454694; PMCID: PMC2835774.
81. Sanborn K B, Rak G D, Mentlik A N, Banerjee P P, Orange J S. Analysis of the N K cell immunological synapse. Methods Mol Biol. 2010; 612:127-48. Epub 2009 Dec. 25. doi: 10.1007/978-1-60761-362-6_9. PubMed PMID: 20033638.
82. Santarossa C C, Mickolajczyk K J, Steinman J B, Urnavicius L, Chen N, Hirata Y, Fukase Y, Coudray N, Ekiert D C, Bhabha G, Kapoor T M. Targeting allostery in the Dynein motor domain with small molecule inhibitors. Cell Chem Biol. 2021; 28 (10): 1460-73 e15. Epub 2021 May 21. doi: 10.1016/j.chembiol.2021.04.024. PubMed PMID: 34015309; PMCID: PMC8542630.
83. Seyfoori A, Barough M S, Amereh M, Jush B K, Lum J J, Akbari M. Bioengineered tissue models for the development of dynamic immuno-associated tumor models and high-throughput immunotherapy cytotoxicity assays. Drug Discov Today. 2021 February; 26 (2): 455-473.
84. Smeland S, Bielack S S, Whelan J, Bernstein M, Hogendoorn P, Krailo M D, Gorlick R, Janeway K A, Ingleby F C, Anninga J, Antal I, Arndt C, Brown K L B, Butterfass-Bahloul T, Calaminus G, Capra M, Dhooge C, Eriksson M, Flanagan A M, Friedel G, Gebhardt M C, Gelderblom H, Goldsby R, Grier H E, Grimer R, Hawkins D S, Hecker-Nolting S, Sundby Hall K, Isakoff M S, Jovic G, Kuhne T, Kager L, von Kalle T, Kabickova E, Lang S, Lau C C, Leavey P J, Lessnick S L, Mascarenhas L, Mayer-Steinacker R, Meyers P A, Nagarajan R, Randall R L, Reichardt P, Renard M, Rechnitzer C, Schwartz C L, Strauss S, Teot L, Timmermann B, Sydes M R, Marina N. Survival and prognosis with osteosarcoma: outcomes in more than 2000 patients in the EURAMOS-1 (European and American Osteosarcoma Study) cohort. Eur J Cancer. 2019; 109:36-50. Epub 2019 Jan. 28. doi: 10.1016/j.ejca.2018.11.027. PubMed PMID: 30685685; PMCID: PMC6506906.
85. Smrke A, Anderson P M, Gulia A, Gennatas S, Huang P H, Jones R L. Future Directions in the Treatment of Osteosarcoma. Cells. 2021; 10 (1). Epub 2021 Jan. 21. doi: 10.3390/cells10010172. PubMed PMID: 33467756; PMCID: PMC7829872.
86. Stabile H, Carlino C, Mazza C, Giliani S, Morrone S, Notarangelo L D, Notarangelo L D, Santoni A, Gismondi A. Impaired N K-cell migration in WAS/XLT patients: role of Cdc42/WASp pathway in the control of chemokine-induced beta2 integrin high-affinity state. Blood. 2010; 115 (14): 2818-26. Epub 2010 Feb. 5. doi: 10.1182/blood-2009-07-235804. PubMed PMID: 20130240; PMCID: PMC2854428.
87. Steinman J B, Santarossa C C, Miller R M, Yu L S, Serpinskaya A S, Furukawa H, Morimoto S, Tanaka Y, Nishitani M, Asano M, Zalyte R, Ondrus A E, Johnson A G, Ye F, Nachury M V, Fukase Y, Aso K, Foley M A, Gelfand V I, Chen J K, Carter A P, Kapoor T M. Chemical structure-guided design of dynapyrazoles, cell-permeable dynein inhibitors with a unique mode of action. Elife. 2017;6. Epub 2017 May 20. doi: 10.7554/eLife.25174. PubMed PMID: 28524820; PMCID: PMC5478271.
88. Sterner R C, Sterner R M. CAR-T cell therapy: current limitations and potential strategies. Blood Cancer J. 2021; 11 (4): 69. Epub 2021 Apr. 8. doi: 10.1038/s41408-021-00459-7. PubMed PMID: 33824268; PMCID: PMC8024391.
89. Talbot L J, Chabot A, Funk A, Nguyen P, Wagner J, Ross A, Tillman H, Davidoff A, Gottschalk S, DeRenzo C. A Novel Orthotopic Implantation Technique for Osteosarcoma Produces Spontaneous Metastases and Illustrates Dose-Dependent Efficacy of B7-H3-CAR T Cells. Front Immunol. 2021; 12:691741. Epub 2021 Jul. 3. doi: 10.3389/fimmu.2021.691741. PubMed PMID: 34211478; PMCID: PMC8239305.
90. Tang B L. A unique SNARE machinery for exocytosis of cytotoxic granules and platelets granules. Mol Membr Biol. 2015;32 (4): 120-6. Epub 2015 Oct. 29. doi: 10.3109/09687688.2015.1079934. PubMed PMID: 26508555.
91. Titov A, Zmievskaya E, Ganeeva I, Valiullina A, Petukhov A, Rakhmatullina A, Miftakhova R, Fainshtein M, Rizvanov A, Bulatov E. Adoptive Immunotherapy beyond CAR T-Cells. Cancers (Basel). 2021; 13 (4). Epub 2021 Mar. 7. doi: 10.3390/cancers13040743. PubMed PMID: 33670139; PMCID: PMC7916861.
92. Trambas C M, Griffiths G M. Delivering the kiss of death. Nat Immunol. 2003; 4 (5): 399-403. Epub 2003 Apr. 30. doi: 10.1038/ni0503-399. PubMed PMID: 12719728.
93. Tuli A, Thiery J, James A M, Michelet X, Sharma M, Garg S, Sanborn K B, Orange J S, Lieberman J, Brenner M B. Arf-like GTPase Arl8b regulates lytic granule polarization and natural killer cell-mediated cytotoxicity. Mol Biol Cell. 2013; 24 (23): 3721-35. Epub 2013 Oct. 4. doi: 10.1091/mbc.E13-05-0259. PubMed PMID: 24088571; PMCID: PMC3842998.
94. Vargas-Hernandez A, Mace E M, Zimmerman O, Zerbe C S, Freeman A F, Rosenzweig S, Leiding J W, Torgerson T, Altman M C, Schussler E, Cunningham-Rundles C, Chinn I K, Carisey A F, Hanson I C, Rider N L, Holland S M, Orange J S, Forbes L R. Ruxolitinib partially reverses functional natural killer cell deficiency in patients with signal transducer and activator of transcription 1 (STAT1) gain-of-function mutations. J Allergy Clin Immunol. 2018; 141 (6): 2142-55 e5. Epub 2017 Nov. 8. doi: 10.1016/j.jaci.2017.08.040. PubMed PMID: 29111217; PMCID: PMC5924437.
95. Viswanath D I, Mace E M, Hsu H T, Orange J S. Quantification of natural killer cell polarization and visualization of synaptic granule externalization by imaging flow cytometry. Clin Immunol. 2017; 177:70-5. Epub 2016 Mar. 8. doi: 10.1016/j.clim.2016.03.004. PubMed PMID: 26948929; PMCID: PMC5010793.
96. Watkin L B, Jessen B, Wiszniewski W, Vece T J, Jan M, Sha Y, Thamsen M, Santos-Cortez R L, Lee K, Gambin T, Forbes L R, Law C S, Stray-Pedersen A, Cheng M H, Mace E M, Anderson M S, Liu D, Tang L F, Nicholas S K, Nahmod K, Makedonas G, Canter D L, Kwok P Y, Hicks J, Jones K D, Penney S, Jhangiani S N, Rosenblum M D, Dell S D, Waterfield M R, Papa F R, Muzny D M, Zaitlen N, Leal S M, Gonzaga-Jauregui C, Baylor-Hopkins Center for Mendelian G, Boerwinkle E, Eissa N T, Gibbs R A, Lupski J R, Orange J S, Shum A K. COPA mutations impair E R-Golgi transport and cause hereditary autoimmune-mediated lung disease and arthritis. Nat Genet. 2015;47 (6): 654-60. Epub 2015 Apr. 22. doi: 10.1038/ng.3279. PubMed PMID: 25894502; PMCID: PMC4513663.
97. Wilton K M, Billadeau D D. VASP Regulates N K Cell Lytic Granule Convergence. J Immunol. 2018; 201 (10): 2899-909. Epub 2018 Oct. 5. doi: 10.4049/jimmunol. 1800254. PubMed PMID: 30282752; PMCID: PMC6317751.
98. Wolf N K, Kissiov D U, Raulet D H. Roles of natural killer cells in immunity to cancer, and applications to immunotherapy. Nat Rev Immunol. 2022. Epub 2022 Jun. 1. doi: 10.1038/s41577-022-00732-1. PubMed PMID: 35637393.
99. Zhang M, March M E, Lane W S, Long E O. A signaling network stimulated by beta2 integrin promotes the polarization of lytic granules in cytotoxic cells. Sci Signal. 2014; 7 (346): ra96. Epub 2014 Oct. 9. doi: 10.1126/scisignal.2005629. PubMed PMID: 25292215; PMCID: PMC4205566.

What is claimed is:

1. A method of inducing multidirectional degranulation by cytotoxic effector cells in a tumor microenvironment comprising a target tumor cell and bystander cells, the method comprising:
inhibiting the function of at least one vesicle modifying protein in the cytotoxic effector cells to produce inhibited cytotoxic effector cells; and
providing the inhibited cytotoxic effector cells to the tumor microenvironment, whereby bystander cells and the target tumor cell are killed.

2. The method of claim 1, wherein the at least one vesicle modifying protein is selected from the group consisting of: a trans-Golgi golgin, Brefeldin A-Inhibited Guanine Nucleotide-Exchange Protein 1 (BIG1), VASP, ARL1, Rab Geranyl-Geranyltransferase Subunit Alpha (RABGGTA), and a cytoskeleton-related protein.

3. The method of claim 2, wherein:
the trans-Golgi golgin is selected from the group consisting of: GCC1, GCC2, Golgin-97, and Golgin-245; and
the cytoskeleton-related protein is selected from the group consisting of: a septin, HAP40, CCDC84, HKRP3, and beta actin (ACTB).

4. The method of claim 2, further comprising administering to the cytotoxic effector cells a small molecule inhibitor prior to providing the inhibited cytotoxic effector cells to the tumor microenvironment, wherein the small molecule inhibitor is selected from the group consisting of: Ciliobrevin-D, Dynapyrazole A, Compound 3016, Compound 5814, Compound 7779, Compound 3977, and a derivative thereof.

5. The method of claim 2, wherein the function of at least one vesicle modifying protein is inhibited by knocking down, knocking out, or knocking in expression of the at least one vesicle modifying protein in the cytotoxic effector cells.

6. The method of claim 5, wherein expression of the at least one vesicle modifying protein in the cytotoxic effector cells is knocked out using CRISPR-mediated genome editing of a trans-Golgi golgin, BIG1, Septin-7, HAP40, CCDC84, HKRP3, ARL1, RABGGTA, or ACTB.

7. The method of claim 1, wherein the function of at least one vesicle modifying protein is inhibited by administering a small molecule inhibitor selected from the group consisting of: 1-oleoyl lysophosphatidic acid (LPA), CFI-400945, Centrinone, Compound 5814, Compound 7779, Compound 3977, and a derivative thereof.

8. The method of claim 1, wherein the tumor microenvironment is that of a solid tumor.

9. The method of claim 8, wherein the solid tumor is osteosarcoma.

10. The method of claim 1, wherein the tumor microenvironment is that of a lymphoma.

11. A method of treating a tumor in a subject, the method comprising:
providing cytotoxic effect cells comprising chimeric antigen receptors specific to the tumor;
inhibiting the function of at least one vesicle modifying protein in the cytotoxic effector cells to produce inhibited cytotoxic effector cells, wherein the at least one protein is selected from the group consisting of: a trans-Golgi golgin, Brefeldin A-Inhibited Guanine Nucleotide-Exchange Protein 1 (BIG1), HKRP3, VASP, ARL1, Rab Geranyl-Geranyltransferase Subunit Alpha (RABGGTA), and a cytoskeleton-related protein; and
administering the inhibited cytotoxic effector cells to the subject.

12. The method of claim 11, wherein:
the trans-Golgi golgin is selected from the group consisting of: GCC1, GCC2, Golgin-97, and Golgin-245; and/or
the cytoskeleton-related protein is selected from the group consisting of: a septin, HAP40, CCDC84, HKRP3, and beta actin (ACTB).

13. The method of claim 11, further comprising administering to the cytotoxic effector cells a small molecule inhibitor prior to administering the inhibited cytotoxic effector cells to the subject, wherein the small molecule inhibitor is selected from the group consisting of: Ciliobrevin-D, Dynapyrazole A, Compound 3016, 1-oleoyl lysophosphatidic acid (LPA), CFI-400945, Compound 5814, Compound 7779, Compound 3977, and a derivative thereof.

14. The method of claim 11, wherein the function of at least one vesicle modifying protein is inhibited by knocking down, knocking in, or knocking out expression of the at least one vesicle modifying protein in the cytotoxic effector cells.

15. The method of claim 14, wherein expression of the at least one vesicle modifying protein in the cytotoxic effector cells is knocked out using CRISPR-mediated genome editing of a trans-Golgi golgin, BIG1, Septin-7, HAP40, CCDC84, HKRP3, ARL1, RABGGTA, or ACTB.

16. A composition comprising inhibited cytotoxic effector cells, wherein lytic granule dispersion in the inhibited cytotoxic effector cells is increased compared to lytic granule dispersion in uninhibited cytotoxic effector cells.

17. The composition of claim 16, wherein the inhibited cytotoxic effector cells have reduced function of at least one vesicle modifying protein compared to uninhibited cytotoxic effector cells, wherein the at least one vesicle modifying protein is selected from the group consisting of: a trans-Golgi golgin, Brefeldin A-Inhibited Guanine Nucleotide-Exchange Protein 1 (BIG1), VASP, ARL1, Rab Geranyl-Geranyltransferase Subunit Alpha (RABGGTA), and a cytoskeleton-related protein.

18. The composition of claim 17, wherein:
the inhibited cytotoxic effector cells have at least one gene knocked out or knocked down, wherein the at least one gene is selected from the group consisting of: GCC1, GCC2, Golgin-97, Golgin-245, Septin-7, HAP40, CCDC84, HKRP3, VASP, ARL1, Rab Geranyl-Geranyltransferase Subunit Alpha (RABGGTA), and beta actin (ACTB);
the at least one gene is knocked out with a gRNA that hybridizes to the gene and a Cas endonuclease;
the at least one gene is knocked down with an shRNA construct for knocking down expression of the at least one gene.

19. The composition of claim 17, wherein the inhibited cytotoxic effector cells express a mutated vesicle modifying protein thereby having reduced function of at least one vesicle modifying protein compared to uninhibited cytotoxic effector cells and the inhibited cytotoxic effector cells have been transfected with a plasmid encoding the mutated vesicle modifying protein.

20. The composition of claim 16, wherein the inhibited cytotoxic effector cells have been treated with a small molecule inhibitor selected from the group consisting of: 1-oleoyl lysophosphatidic acid (LPA), CFI-400945, Centrinone, Compound 5814, Compound 7779, Compound 3977, and a derivative thereof.
